(19) United States
(12) Patent Application Publication
(10) Pub. No.: US 2011/0046349 A1
(43) Pub. Date: Feb. 24, 2011

(54) PROCESS FOR THE PRODUCTION OF EXENATIDE AND OF AN EXENATIDE ANALOGUE

(76) Inventors: Matthieu Giraud, Sion (CH); Anne-Sophie Droz, Sierre (CH); Stéphane Varray, Sierre (CH); El Djouhar Rekai, Braine l'Alleud (BE); Marie-Hèléne Brichard, Gembloux (BE); Daniel Latassa, Veyras (CH); Christine Devijver, Brussels (BE); Pascal Gilles, Feluy (BE); Jeanne-Marie Cauvin, lttre (BE); Fernando Albericio, Barcelona (ES); Marta Paradis Bas, Barcelona (ES)

Correspondence Address: Ronald J. Baron, Esq.;Hoffmann & Baron, LLP, 6900 Jericho Turnpike, Syosset, NY, 11791, US

(21) Appl. No.: 12/836,411

(22) Filed: Jul. 14, 2010

Related U.S. Application Data
(60) Provisional application No. 61/240,817, filed on Sep. 9, 2009.
(60) Provisional application No. 61/287,015, filed on Dec. 16, 2009.

(30) Foreign Application Priority Data
Jul. 15, 2009 (EP) 09009219.8
Dec. 14, 2009 (EP) 09015428.7

(51) Int. Cl. C07K 1/06; C07K 2/00

(52) U.S. Cl. 530/337; 530/300

(57) ABSTRACT

Exenatide, a polypeptide having the 39 amino acid sequence

H-¹His-Gly-Glu-Gly-⁵Thr-Phe-Thr-Ser-Asp-¹⁰Leu-Ser-
Lys-Gln-Met-¹⁵Glu-Glu-Glu--Ala-Val-²⁰Arg-Leu-Phe-
Ile-Glu-²⁵Trp-Leu-Lys-Asn-Gly-³⁰Gly-Pro-Ser-Ser-
Gly-³⁵Ala--Pro-Pro-Pro-Ser-NH₂,

respectively its 44-mer analogue

H-¹His-Gly-Glu-Gly-⁵Thr-Phe-Thr-Ser-Asp-¹⁰Leu-Ser-
Lys-Gln-Met-¹⁵Glu-Glu-Glu--Ala-Val-²⁰Arg-Leu-Phe-
Ile-Glu-²⁵Trp-Leu-Lys-Asn-Gly-³⁰Gly-Pro-Ser-Ser-
Gly-³⁵Ala--Pro-Pro-Ser-Lys-⁴⁰Lys-Lys-Lys-Lys-Lys-
NH₂

is prepared via a convergent four-fragment synthesis strategy from the fragments comprising the amino acid positions 1-10, 11-21, 22-29 and 30-39, respectively 30-44.

### CROSS-REFERENCE TO RELATED APPLICATIONS AND CLAIM OF PRIORITY

This application claims the benefit of priority from European Patent Application No. 09009219.8 filed Jul. 15, 2009, European Patent Application No. 09015428.7 filed Dec. 14, 2009, U.S. patent application Ser. No. 61/240,817 filed Sep. 9, 2009, and U.S. patent application Ser. No. 61/287,015 filed Dec. 16, 2009, the disclosures of which are incorporated herein by reference.

### INCORPORATION OF SEQUENCE LISTING

Incorporated herein by reference in its entirety is a Sequence Listing disclosed on a computer-readable ASCII text file titled “SequenceListing.txt”, created on Jul. 14, 2010. The sequence.txt file is 7.75 KB. Also submitted herewith is an identical sequence listing mentioned above in a portable document format (pdf) file entitled “SequenceListing.pdf”.

### BACKGROUND OF THE INVENTION

The invention relates to a novel convergent synthesis of exenatide which is a 39-mer peptide of formula

(SEQ ID NO 1)
H-¹His-Gly-Glu-Gly-⁵Thr-Phe-Thr-Ser-Asp-¹⁰Leu-Ser-
Lys-Gln-Met-¹⁵Glu-Glu-Glu--Ala-Val-²⁰Arg-Leu-Phe-
Ile-Glu-²⁵Trp-Leu-Lys-Asn-Gly-³⁰Gly-Pro-Ser-Ser-
Gly-³⁵Ala--Pro-Pro-Pro-Ser-NH₂
(Ia)

and of its 44-mer analogue of formula

(SEQ ID NO 2)
H-¹His-Gly-Glu-Gly-⁵Thr-Phe-Thr-Ser-Asp-¹⁰Leu-Ser-
Lys-Gln-Met-¹⁵Glu-Glu-Glu--Ala-Val-²⁰Arg-Leu-Phe-
Ile-Glu-²⁵Trp-Leu-Lys-Asn-Gly-³⁰Gly-Pro-Ser-Ser-
Gly-³⁵Ala--Pro-Pro-Ser-Lys-⁴⁰Lys-Lys-Lys-Lys-Lys-
NH₂
(Ib)

The invention further relates to several side chain-protected peptides as intermediates in the synthesis of the peptides of formula Ia and Ib.

Exenatide (synonym is exendin 4) and the exenatide analogue of formula Ib are bioactive polypeptides and act as glucagon-like peptide-1 (GLP-1) agonists usable for the treatment of type 2 diabetes. Exenatide is the drug substance of the commercially available drug product Byetta®.

Known syntheses of exenatide (WO-A-2008/109079, WO-A-2006/119388) apply a classical linear approach. Single amino acid residues are covalently coupled to a growing peptide chain which is covalently linked to a solid resin support (SPPS). WO-A-2008/109079 discloses a stepwise Fmoc/tBu SPPS method characterized in an extra purification step of semi-protected peptide. The peptide is supported on Rink amide resin and is loaded only in small scale (100 g). The extra purification step indicates that the linear approach for producing exenatide suffers from side-reactions. Such side-reactions often arise in SPPS by misincorporation, double-hits of single amino acids and/or racemization and lead to side-products which have a structure very similar to that of the target peptide. Purification is therefore awkward and results in loss of yield. Especially longer peptides are prone to adopt an irregular conformation while still attached to the solid support, which makes it even more difficult to add additional amino acids to the growing chain. Therefore, this problem increases as the length of the peptide increases.

WO-A-2006/119388 describes the preparation of the exenatide sequence applying the Fmoc/tBu SPPS protocol and cleavage from the resin to form the free acid at its C-terminus, followed by amide formation to provide exenatide. WO-A-2006/119388 is silent about the amounts of starting materials and the yield of exenatide and its precursor, which also indicates that this approach is not suitable for production of exenatide on large scale with good purity.

### DESCRIPTION OF THE INVENTION

It is an object of the present invention to provide a more efficient synthesis of exenatide and its analogue of formula Ib that overcomes the known drawbacks of linear solid phase synthesis and is suitable for the production on an industrial scale. This object has been achieved by the process according to claim 1, by the peptide fragments of claim 10, and by the use of the peptide fragments of claim 15. Preferred embodiments constitute the subject-matter of dependent claims.

The present invention relates to a process following a convergent approach, i.e. individual fragments are synthesized separately and then coupled in solution phase to build the desired peptide. The challenge of convergent synthesis is to find suitable fragments and their coupling order for overcoming the known drawbacks of convergent synthesis. These drawbacks are solubility problems during coupling and isolation, lower reaction rates compared to SPPS and a much higher racemization risk of the C-terminal fragment during coupling. Exenatide consists of thirty-nine amino acid residues and the exenatide analogue of formula Ib consists of forty-four amino acid residues so that a huge number of possible fragments and coupling orders exists.

Applicant has surprisingly found a suitable strategy for the preparation of exenatide

(SEQ ID NO 1)
H-¹His-Gly-Glu-Gly-⁵Thr-Phe-Thr-Ser-Asp-¹⁰Leu-Ser-
Lys-Gln-Met-¹⁵Glu-Glu-Glu--Ala-Val-²⁰Arg-Leu-Phe-
Ile-Glu-²⁵Trp-Leu-Lys-Asn-Gly-³⁰Gly-Pro-Ser-Ser-
Gly-³⁵Ala--Pro-Pro-Pro-Ser-NH₂
(Ia),

respectively for its 44-mer analogue of formula

(SEQ ID NO 2)
H-¹His-Gly-Glu-Gly-⁵Thr-Phe-Thr-Ser-Asp-¹⁰Leu-Ser-
Lys-Gln-Met-¹⁵Glu-Glu-Glu--Ala-Val-²⁰Arg-Leu-Phe-
Ile-Glu-²⁵Trp-Leu-Lys-Asn-Gly-³⁰Gly-Pro-Ser-Ser-
Gly-³⁵Ala--Pro-Pro-Ser-Lys-⁴⁰Lys-Lys-Lys-Lys-Lys-
NH₂
(Ib),

which comprises the specific coupling of four peptide fragments, namely of the fragments comprising the amino acid positions 1-10,11-21,22-29 and 30-39, respectively 30-44, of exenatide and of its analogue of formula Ib.

In particular, one aspect of the invention is a process in solution phase comprising the steps of
(a) reacting a side chain-protected peptide of formula

(II),
(SEQ ID NO 3)
P1-Phe-Ile-Glu-²⁵Trp-Leu-Lys-Asn-Gly-OH

wherein P1 is an carbamate-type protecting group,
in the following abbreviated with P1-[22-29]-OH;
with a side chain-protected peptide of formula

(IIIa),
(SEQ ID NO 4)
H-³⁰Gly-Pro-Ser-Ser-Gly-³⁵Ala-Pro-Pro-Pro-Ser-NH₂

in the following abbreviated with H-[30-39]-NH₂,
respectively

(IIIb),
(SEQ ID NO 5)
H-³⁰Gly-Pro-Ser-Ser-Gly-³⁵Ala-Pro-Pro-Ser-Lys-
⁴⁰Lys-Lys-Lys-Lys-Lys-NH₂

in the following abbreviated with H-[30-44]-NH₂;
to produce a side chain-protected peptide of formula

(IVa),
(SEQ ID NO 6)
P1-Phe-Ile-Glu-²⁵Trp-Leu-Lys-Asn-Gly-³⁰Gly-Pro-
Ser-Ser-Gly-³⁵Ala-Pro-Pro--Pro-Ser-NH₂

in the following abbreviated with P1-[22-39]-NH₂;
respectively

(IVb),
(SEQ ID NO 7)
P1-Phe-Ile-Glu-²⁵Trp-Leu-Lys-Asn-Gly-³⁰Gly-Pro-
Ser-Ser-Gly-³⁵Ala-Pro-Pro--Ser-Lys-⁴⁰Lys-Lys-Lys-
Lys-Lys-NH₂

in the following abbreviated with P1-[22-44]-NH₂;
wherein P1 is as defined above,
(b) removing the N-terminal protecting group P1 of the side chain-protected peptide of formula IVa, respectively IVb, to produce the corresponding N-terminally-deprotected, side chain-protected peptide,
(c) reacting the N-terminally-deprotected, side chain-protected peptide produced in step (b) with a side chain-protected peptide of formula

(V),
(SEQ ID NO 8)
P2-Ser-Lys-Gln-Met-¹⁵Glu-Glu-Glu-Ala-Val-²⁰Arg-
Leu-OH

wherein P2 is an carbamate-type protecting group,
in the following abbreviated with P2-[11-21]-OH,
to produce a side chain-protected peptide of formula

(VIa),
(SEQ ID NO 9)
P2-Ser-Lys-Gln-Met-¹⁵Glu-Glu-Glu-Ala-Val-²⁰Arg-
Leu-Phe-Ile-Glu-²⁵Trp-Leu--Lys-Asn-Gly-³⁰Gly-
Pro-Ser-Ser-Gly-³⁵Ala-Pro-Pro-Pro-Ser-NH₂

in the following abbreviated with P2-[11-39]-NH₂,
respectively

(VIb),
(SEQ ID NO 10)
P2-Ser-Lys-Gln-Met-¹⁵Glu-Glu-Glu-Ala-Val-²⁰Arg-
Leu-Phe-Ile-Glu-²⁵Trp-Leu--Lys-Asn-Gly-³⁰Gly-
Pro-Ser-Ser-Gly-³⁵Ala-Pro-Pro-Ser-Lys-⁴⁰Lys-
Lys-Lys-Lys--Lys-NH₂

in the following abbreviated with P2-[11-44]-NH₂, wherein P2 is as defined above,
(d) removing the N-terminal protecting group P2 of the side chain-protected peptide of formula VIa, respectively VIb, to produce the corresponding N-terminally-deprotected, side chain-protected peptide,
(e) reacting the N-terminally-deprotected, side chain-protected peptide produced in step (d) with a side chain-protected peptide of formula

(VII),
(SEQ ID NO 11)
P3-¹His-Gly-Glu-Gly-⁵Thr-Phe-Thr-Ser-Asp-¹⁰Leu-OH

in following abbreviated with P3-[1-10]-OH;
wherein P3 is an carbamate-type protecting group,
to produce a side chain-protected peptide of formula

(VIIIa),
(SEQ ID NO 1)
P3-¹His-Gly-Glu-Gly-⁵Thr-Phe-Thr-Ser-Asp-¹⁰Leu-
Ser-Lys-Gln-Met-¹⁵Glu-Glu--Glu-Ala-Val-²⁰Arg-
Leu-Phe-Ile-Glu-²⁵Trp-Leu-Lys-Asn-Gly-³⁰Gly-
Pro-Ser-Ser--Gly-³⁵Ala-Pro-Pro-Pro-Ser-NH₂

respectively

(VIIIb),
(SEQ ID NO 2)
P3-¹His-Gly-Glu-Gly-⁵Thr-Phe-Thr-Ser-Asp-¹⁰Leu-
Ser-Lys-Gln-Met-¹⁵G1u-Glu--Glu-Ala-Val-²⁰Arg-Leu-
Phe-Ile-Glu-²⁵Trp-Leu-Lys-Asn-Gly-³⁰Gly-Pro-Ser-
Ser--Gly-³⁵Ala-Pro-Pro-Ser-Lys-⁴⁰Lys-Lys-Lys-Lys-
Lys-NH₂

wherein P3 is as defined above,
(f) removing the N-terminal and side chain protecting groups of the side chain-protected peptide of formula VIIIa, respectively VIIIb, to produce the peptide of formula Ia, respectively Ib.

Here and in the following, the term “carbamate-type protecting group” is to be understood to mean a protecting group which forms an oxycarbonylamino moiety by reaction with the amino group of the peptide. Any known carbamate-type protecting group which suits both the assembling strategy of the fragments and the coupling protocol may be applied. Suitable carbamate-type protecting groups are for example fluoren-9-ylmethoxycarbonyl (Fmoc), tert-butoxycarbonyl (Boc), benzyloxycarbonyl (Z), p-bromobenzyloxycarbonyl [Z(Br)], o-chlorobenzyloxycarbonyl [Z(Cl)], 2-(p-biphenylyl)isopropyloxycarbonyl (Bpoc), allyloxycarbonyl (Alloc), 1-methyl-1-(3,5-dimethoxyphenyl)ethoxycarbonyl (Ddz), p-phenylazobenzyloxycarbonyl (Pz), p-nitrobenzyloxycarbonyl [Z(NO₂)], p-methoxybenzyloxycarbonyl [Z(OMe)] and benz[f]inden-3-ylmethoxycarbonyl (Bimoc).The carbamate-type protecting group chosen for protection of the N-terminus is typically orthogonal to the side chain protecting groups of the peptide, i.e. it may be cleaved by a method that does not affect the side chain protecting groups. The carbamate-type protecting group of the side-chain protected peptide of formula VII, which is the last fragment to be coupled, may be orthogonal or non-orthogonal to its side chain protecting groups. Suitably, it is non-orthogonal to accomplish concomitant deprotection of the N-terminal and side chain protecting groups to produce exenatide, respectively its analogue of formula Ib, in the same step.

In a preferred embodiment, each carbamate protecting group P1, P2 and P3 of the side chain-protected peptides of formula II, IVa, respectively IVb, V, VIa, respectively VIb, VII and VIIIa, respectively VIIIb, is independently selected from the group consisting of fluoren-9-ylmethoxycarbonyl (Fmoc), tert-butoxycarbonyl (Boc) and allyloxycarbonyl (Alloc).

Preferably, P1 and P2 are Fmoc and P3 is Fmoc or Boc, preferably P3 is Boc.

In a more preferred embodiment, the carbamate protecting groups P1 and P2 are Fmoc and the carbamate protecting group P3 is Fmoc or Boc, preferably P3 is Boc, affording the solution phase process which comprises the steps of
(a) reacting a side chain-protected peptide of formula

(II),
(SEQ ID NO 3)
Fmoc-Phe-Ile-Glu-²⁵Trp-Leu-Lys-Asn-Gly-OH

with a side chain-protected peptide of formula

(IIIa),
(SEQ ID NO 4)
H-³⁰Gly-Pro-Ser-Ser-Gly-³⁵Ala-Pro-Pro-Pro-Ser-NH₂

respectively

(IIIb),
(SEQ ID NO 5)
H-³⁰Gly-Pro-Ser-Ser-Gly-³⁵Ala-Pro-Pro-Ser-Lys-
⁴⁰Lys-Lys-Lys-Lys-Lys-NH₂

to produce a side chain-protected peptide of formula

(IVa),
(SEQ ID NO 6)
Fmoc-Phe-lIe-Glu-²⁵Trp-Leu-Lys-Asn-Gly-³⁰Gly-
Pro-Ser-Ser-Gly-³⁵Ala-Pro-Pro--Pro-Ser-NH₂

respectively

(SEQ ID NO 7)
Fmoc-Phe-Ile-Glu-²⁵Trp-Leu-Lys-Asn-Gly-³⁰Gly-Pro-
Ser-Ser-Gly-³⁵Ala-Pro-Pro--Ser-Lys-⁴⁰Lys-Lys-Lys-
Lys-Lys-NH₂
(IVb)

(b) removing the N-terminal protecting group of the side chain-protected peptide of formula IVa, respectively IVb, to produce the corresponding N-terminally-deprotected, side chain-protected peptide,
(c) reacting the N-terminally-deprotected, side chain-protected peptide produced in step (b) with a side chain-protected peptide of formula

(SEQ ID NO 8)
Fmoc-Ser-Lys-Gln-Met-¹⁵Glu-Glu-Glu-Ala-Val-²⁰Arg-
Leu-OH
(V),

to produce a side chain-protected peptide of formula

(SEQ ID NO 9)
Fmoc-Ser-Lys-Gln-Met-¹⁵Glu-Glu-Glu-Ala-Val-²⁰Arg-
Leu-Phe-Ile-Glu-²⁵Trp--Leu-Lys-Asn-Gly-³⁰Gly-Pro-
Ser-Ser-Gly-³⁵Ala-Pro-Pro-Pro-Ser-NH₂
(VIa),

respectively

(SEQ ID NO 10)
Fmoc-Ser-Lys-Gln-Met-¹⁵Glu-Glu-Glu-Ala-Val-²⁰Arg-
Leu-Phe-Ile-Glu-²⁵Trp--Leu-Lys-Asn-Gly-³⁰Gly-Pro-
Ser-Ser-Gly-³⁵Ala-Pro-Pro-Ser-Lys-⁴⁰Lys-Lys-Lys--
Lys-Lys-NH₂
(VIb)

(d) removing the N-terminal protecting group of the side chain-protected peptide of formula VIa, respectively VIb, to produce the corresponding N-terminally-deprotected, side chain-protected peptide,
(e) reacting the N-terminally-deprotected, side chain-protected peptide produced in step (d) with a side chain-protected peptide of formula

(SEQ ID NO 11)
P3-¹His-Gly-Glu-Gly-⁵Thr-Phe-Thr-Ser-Asp-¹⁰Leu-OH
(VII),

wherein P3 is Fmoc or Boc, preferably P3 is Boc,
to produce a side chain-protected peptide of formula

(SEQ ID NO 1)
P3-¹His-Gly-Glu-Gly-⁵Thr-Phe-Thr-Ser-Asp-¹⁰Leu-
Ser-Lys-Gln-Met-¹⁵Glu-Glu--Glu-Ala-Val-²⁰Arg-Leu-
Phe-Ile-Glu-²⁵Trp-Leu-Lys-Asn-Gly-³⁰Gly-Pro-Ser-
Ser--Gly-³⁵Ala-Pro-Pro-Pro-Ser-NH₂
(VIIIa),

respectively

(SEQ ID NO 2)
P3-¹His-Gly-Glu-Gly-⁵Thr-Phe-Thr-Ser-Asp-¹⁰Leu-
Ser-Lys-Gln-Met-¹⁵G1u-Glu--Glu-Ala-Val-²⁰Arg-Leu-
Phe-Ile-Glu-²⁵Trp-Leu-Lys-Asn-Gly-³⁰Gly-Pro-Ser-
Ser--Gly-³⁵Ala-Pro-Pro-Ser-Lys-⁴⁰Lys-Lys-Lys-Lys-
Lys-NH₂
(VIIIb),

wherein P3 is as defined above,
(f) removing the N-terminal and side chain protecting groups of the side chain-protected peptide of formula VIIIa, respectively VIIIb, to produce the peptide of formula Ia, respectively Ib.

In the process according to the invention, the peptides of formula II to VIIIa/VIIIb or any peptide fragments defined in the following text are protected with at least one side chain protecting group. In some cases and depending on the type of reagent used in assembling the peptides, an amino acid residue may not require the presence of a side chain protecting group. Such amino acids typically do not include reactive oxygen, nitrogen or other reactive moiety in the side chain.

Any known side chain protecting group which suits with both the assembling strategy of the fragments and the coupling protocol may be applied. Examples for suitable side chain protecting groups are tert-butyl (tBu), trityl (Trt), 4-methoxytrityl (Mmt), 4-methyl-trityl (Mtt), 3-methyl-3-pentyl (Mpe), benzyl (Bzl), 2,4-dinitrophenyl (Dnp), cyclohexyl (cHex), 4-{N-[1-(4,4-dimethyl-2,6-dioxocyclohexylidene)-3-methylbutyl]amino}benzyl (Dmab), allyl (All), dimethylcyclopropylmethyl (Dmcp), tert-butoxycarbonyl (Boc), o-chlorobenzyloxycarbonyl [Z(Cl)], p-bromobenzyloxycarbonyl [Z(Br)], benzyloxycarbonyl (Z), 1-methyl-1-(3,5-dimethoxyphenyl)ethoxycarbonyl (Ddz), 2,2,4,6,7-pentamethyl-dihydrobenzofuran-5-sulfonyl (Pbf), 1,2-dimethylindole-3-sulfonyl (MIS), 2,2,5,7,8-pentamethylchroman-6-sulfonyl (Pmc), 2,3,6-trimethyl-4-methoxybenzenesulfonyl (Mtr), p-toluenesulfonyl (Tos) and formyl (For).

In the process according to the invention, another suitable side chain protecting group for the Ser residue and/or the Thr residue of the side chain protected fragments IIIa/IIIb to VIIIa/VIIIb or of any peptide fragments defined in the following text is pseudoproline. The term “pseudoproline” is to be understood to mean that the hydroxy function in the side chain of the Ser and/or the Thr residue is protected as a proline-like, acid labile, preferably trifluoroacetic acid labile, oxazolidine ring formed after reaction between the amino group and the side chain hydroxy group of Ser or Thr. The pseudoproline moiety may improve the solubility of the peptide and thus prevent or decreases aggregation, which is advantageous for the process. Particularly, one of the Ser residues in the segment -Pro-Ser-Ser-Gly- of the side chain protected fragment of formula IVa/IVb, may be advantageously protected by the pseudoproline protecting group.

If the carbamate-type protecting group of the N-terminus is Z, [Z(Br)], Pz, [Z(NO₂)] or [Z(OMe)], the side chain protecting group is preferably selected from the group consisting of Pmc, Trt, Bzl, tBu, Z, Boc, Bzl and tBu.

If the carbamate-type protecting group of the N-terminus is Boc or Bpoc, the side chain protecting group is preferably selected from the group consisting of cHex, Z, tBu, Mtr, Tos, Dnp, [Z(Cl)], [Z(Br)], For, Trt and Bzl.

If the carbamate-type protecting group of the N-terminus is Fmoc, Alloc or Ddz, the side chain protecting group is preferably selected from the group consisting of tBu, Trt, Boc, Pbf, Pmc, Dmcp, Bzl, Dmab, Mpe, Mtt, All, pseudoproline, Mmt and MIS.

In a more preferred embodiment of the process of the invention, tBu, Trt, Boc and Pbf are used as side chain protecting groups.

More preferably, the side chain-protected peptides of formula IIa, respectively IIb, to VIIIa, respectively VIIIb, or any side chain protected peptide fragment defined in the following text, are protected with at least one side chain protecting group selected from the group consisting of tert-butyl (tBu), trityl (Trt), tert-butoxycarbonyl (Boc) and 2,2,4,6,7-pentamethyldihydrobenzofuran-5-sulfonyl (Pbf).

The tBu group is a preferred side chain protecting group for the amino acid residues Glu, Asp, Ser and Thr. The Trt group is a preferred side chain protecting group for the amino acid residues Asn, Gln and His. The Boc group is a preferred side chain protecting group for the amino acid residues Lys and Trp. The Pbf group is a preferred side chain protecting group for the amino acid residue Arg. The MIS group is alternatively preferred for the protection of Arg.

The coupling steps (a), (c) and (e) of the process according to the invention, as well as the steps (a-ex), (c-ex) and (f-ex), these three latter steps being defined further down in the text, are performed in solution phase and can be carried out using reaction conditions known in the art of peptide synthesis. Coupling of the respective side chain-protected peptide fragments can be accomplished using in situ coupling reagents, for example phoshonium or uronium coupling reagents, like benzotriazol-1-yloxy-tris(dimethylamino)phosphonium hexafluorophosphate (BOP), benzotriazol-1-yloxy-tris(pyrrolidino)phosphonium hexafluorophosphate (PyBOP), O-(benzotriazol-1-yl)-1,1,3,3-tetramethyluronium hexafluorophosphate (HBTU), O-(6-chlorobenzotriazol-1-yl)-1,1,3,3-tetramethyluronium hexafluorophosphate (HCTU), O-(6-chlorobenzotriazol-1-yl)-1,1,3,3-tetramethyluronium tetrafluoroborate (TCTU), O-(7-azabenzotriazol-1-yl)-1,1,3,3-tetramethyluronium hexafluorophosphate (HATU), O-(7-azabenzotriazol-1-yl)-1,1,3,3-tetramethyluronium tetrafluoroborate (TATU), O-(benzotriazol-1-yl)-1,1,3,3-tetramethyluronium tetrafluoroborate (TBTU), and O-cyano(ethoxycarbonyl)methylenamino]-1,1,3,3-tetramethyluronium tetrafluoroborate (TOTU), or carbodiimide coupling reagents, like diisopropylcarbodiimide (DIC), dicyclohexylcarbodiimide (DCC) and water-soluble carbodiimides (WSCDI) like 1-ethyl-3-(3-dimethylaminopropyl)carbodiimide (EDC). Other coupling techniques use pre-formed active esters, such as hydroxysuccinimide (HOSu) and p-nitrophenol (HONp) esters, pre-formed symmetrical anhydrides, non-symmetrical anhydrides such as N-carboxyanhydrides (NCAs) and acid halides, such as acyl fluoride or acyl chloride. Preferred coupling reagents are phoshonium or uronium coupling reagents, most preferred are TBTU, TOTU or PyBop.

In case coupling is achieved by use of phoshonium or uronium coupling reagents, the reaction mixture of the coupling steps (a), (c) and (e), or of the coupling steps (a-ex), (c-ex) and (e-ex), these three latter steps being defined further down in the text, advantageously contains a base, preferably a tertiary base, which deprotonates the carboxy component, and thus facilitates the in situ reaction. Suitable bases are for example trialkylamines, like N,N-diisopropylethylamine (DIPEA); N,N-dialkylanilines, like N,N-diethylaniline; 2,4,6-trialkylpyridines, like 2,4,6-trimethylpyridine; and N-alkylmorpholines, like N-methylmorpholine. In particular, the reaction mixture advantageously contains DIPEA as a base.

The reaction mixture of the coupling steps (a), (c) and (e), or of the coupling steps (a-ex), (c-ex) and (e-ex), these three latter steps being defined further down in the text, can additionally contain auxiliary nucleophiles as additives due their positive effect in suppressing undesired side reactions. Any known auxiliary nucleophile may be applied. Examples of suitable auxiliary nucleophiles are 1-hydroxybenzotriazole (HOBt), N-hydroxysuccinimide (HOSu), N-hydroxy-3,4-dihydro-4-oxo-1,2,3-benzotriazine (HOOBt), 1-hydroxy-7-azabenzotriazole (HOAt) and ethyl 2-cyano-2-hydroxyiminoacetate (OXYMAPURE®). Also suitable is 6-chloro-1-hydroxybenzotriazole (Cl-HOBt). OXYMAPURE® has proved to be an effective scavenger as racemization is more suppressed compared to benzotriazole-based scavengers. In addition, it is less explosive than e.g. HOBt, so that its handling is advantageous, and, as a further advantage, the coupling progress can be visually monitored by color change. Preferably, the reaction mixtures of the coupling steps additionally contain HOBt or OXYMAPURE®. Most preferably, the reaction mixtures additionally contain HOBt.

In a preferred embodiment, the coupling mixture of the coupling steps (a), (c) and (e) or of the coupling steps (a-ex), (c-ex) and (e-ex), these three latter steps being defined further down in the text, is selected from the group consisting of TBTU/HOBt/DIPEA, TOTU/HOBt/DIPEA and PyBop/HOBt/DIPEA.

As solvent of the coupling steps (a), (c) and (e), or of the coupling steps (a-ex), (c-ex) and (e-ex), these three latter steps being defined further down in the text, any inert liquid solvent which can dissolve the reactants may be used. Applicable coupling solvents are water-miscible solvents like dimethyl sulfoxide (DMSO), chloroform, dioxane, tetrahydrofuran (THF), 1-methyl-2-pyrrolidone (NMP), N,N-dimethylformamide (DMF), N,N-dimethylacetamide (DMA), or any mixture thereof; non water-miscible solvents like dichloromethane (DCM), ethyl acetate or any mixture thereof; and any suitable mixture between water-miscible and non water-miscible solvents. Preferred solvents are NMP, DMF and any mixtures thereof.

Preferably, at least in one of the steps (a) to (e), or in one of the coupling steps (a-ex), (b-ex), (c-ex), (d-ex) and (e-ex), these five latter steps being defined further down in the text, the solvent used is 1-methyl-2-pyrrolidone, N,N-dimethylformamide or a mixture thereof.

Typically, the side chain protected peptides obtained after the coupling steps (a), (c) and (e), or after the coupling steps (a-ex), (c-ex) and (e-ex), these three latter steps being defined further down in the text, are isolated before subjecting to the following deprotection step. Applicant has surprisingly found that this isolation can be waived obtaining similar yields and without negative effect in purity. For example, the purity of H-[22-39]-NH2 with Fmoc-[22-39]-NH₂ isolation is 87% (see Example 9) compared to a even better purity of 88% without isolation (see Example 14). This is surprising as normally isolation is essential to remove side products which often react in the following steps, and thus lower the purity of the target peptide. The finding has therefore a positive effect on costs and time for the overall process and typically announces a higher yield of the N-terminally-deprotected peptides, as isolation usually entails loss of product.

Another aspect is that, when isolating the side chain protected peptides obtained after the coupling steps (a), (c) and (e), or after the coupling steps (a-ex), (c-ex) and (e-ex), these three latter steps being defined further down in the text, after precipitation, it may take a long time till filtration is completed. This is not so relevant in laboratory scale but is of high importance on commercial scale, where clogging of filters should be avoided and a high throughput is desired. Therefore it is also beneficial if isolation, and thus filtration, can be suppressed.

In a preferred embodiment of the process according to the invention, after at least one of the steps (a), (c) and (e), or after at least one of the coupling steps (a-ex), (c-ex) and (e-ex), these three latter steps being defined further down in the text, the side chain-protected peptide thus obtained is not isolated before continuing with the following step. In a more preferred embodiment, after step (a) or after step (a-ex) the side chain-protected peptide thus obtained is not isolated before continuing with the following step.

N-terminal deprotection of the steps (b), (d) and (f), or of the coupling steps (b-ex), (d-ex) and (f-ex), these three latter steps being defined further down in the text, can be carried out using reaction conditions known in the art of peptide synthesis and depends on the nature of the carbamate-type protecting group. In case the carbamate-type protecting group is Boc, deprotection is suitably accomplished by acid, preferably by trifluoroacetic acid, which is preferably applied neat or as a mixture with an inert solvent, advantageously as a mixture with dichloromethane (DCM). In case the carbamate-type protecting group is Fmoc, N-terminal deprotection can be achieved by reaction with a base, favorably with a secondary amine such as piperidine or diethylamine. Typically, N-terminal deprotection is carried out in a solvent which can be any inert solvent like dichloromethane (DCM), dimethylformamide (DMF) or 1-methyl-2-pyrrolidone (NMP). Preferably, deprotection of the N-terminal Fmoc group is carried out by use of diethylamine in N,N-dimethylformamide (DMF) or dichloromethane (DCM) or any mixture thereof.

The side chain protecting groups are typically retained throughout fragment assembly and throughout the solution phase coupling reactions. Generally after the last solution phase-coupling-step, the side chain protecting groups are deprotected. This reaction can be carried out under conditions generally known in peptide chemistry. In case different types of side chain protecting groups are chosen, they may be cleaved successively. Advantageously, they are cleaved simultaneously, and more advantageously concomitant with the carbamate-type protecting group at the N-terminus of the peptide (global deprotection). Typically, the removal of side chain protecting groups by global deprotection employs a deprotection solution that includes an acidolytic agent to cleave the side chain protecting groups. Commonly used acidolytic reagents for global deprotection include hydrogen acids like trifluoroacetic acid (TFA), hydrochloric acid, liquid hydrofluoric acid or trifluoromethanesulfonic acid, and Lewis acids like trifluoroborate diethyl ether adduct or trimethylsilylbromid. As during deprotection highly reactive carbocations are generated, the deprotection mixture advantageously contains scavangers such as dithiothreitol (DTT), ethanedithiol (EDT), dimethylsulfide (DMS), triisopropylsilane (TIS), water, anisole or p-cresol. In a preferred embodiment, the N-terminal and side chain protecting groups in the final deprotection step (f) or step (f-ex) are deprotected in the same step with neat TFA, i.e. without further solvent, in the presence of the scavengers TIS, EDT, water, DMS and ammonium iodide.

The crude product obtained after step (f) or step (f-ex) can be purified by conventional methods, e.g. with preparative HPLC or countercurrent distribution. Purification steps may be repeated.

The same applies to the intermediates obtained after steps (a) to (e), or after steps (a-ex), (b-ex), (c-ex), (d-ex) or (e-ex), these five latter steps being defined further down in the text, if purification is required.

The final peptides of formula Ia/Ib can be isolated according to known isolation methods in peptide chemistry, such as precipitation, freeze-drying and spray-drying. Spray-drying is a known and commonly applied technique for the isolation of non-peptidic organic molecules. This technique has been explored for use with peptides as well. However on spray-drying, peptides and small proteins typically show loss of activity and increased aggregation. In addition, the peptides often partially degrade under the high temperature conditions employed for many spray-drying protocols. It has been suprisingly found that spray-drying of exenatide works well without loss of activity and with excellent purity.

Therefore, in a preferred embodiment, in step (f) or step (f-ex), this latter step being defined further down in the text, after removing the N-terminal and side chain protecting groups of the side chain protected peptide of formula VIIIa/VIIIb, the peptide thus obtained is spray-dried to produce the peptide of formula Ia/Ib. Typically, spray-drying is carried out with a peptide concentration of 30-60 g/L, preferably of 40-50 g/L, in a inert solvent, preferably in a mixture of water/acetic acid/acetonitrile; with a flow rate (feed) of 1.8-2.6 kg/h, preferably of 2.0-2.4 kg/h; with a nitrogen temperature so that the nitrogen gas is dry, preferably of 160-180° C., most preferably of 165-175° C.; and with a nitrogen flow rate of 900-1300 L/min, preferably of 1000-1200 L/min.

The side chain-protected peptide fragments II, IIIa/IIIb, V and VII, or the side chain protected peptide fragments (A), (B), (CL) and (CR), as defined further down in the text, can be prepared using conventional peptide synthesis methods, e.g. solution phase synthesis (SPS), solid phase peptide synthesis (SPPS) or a combination of SPS and SPPS (mixed synthesis). In a preferred embodiment, at least one of the side chain-protected peptides of formula II IIIa/IIIb, V, VII, or at least one of the side chain protected peptide fragments (A), (B), (CL) and (CR), as defined further down in the text, is prepared by SPPS. Particularly, the side chain-protected peptides of formula II, IIIa/IIIb, V, VII, or the side chain protected peptide fragments (A), (B), (CL) and (CR), as defined further down in the text, are prepared by SPPS. These SPPS preparations are preferably done in a precedent process.

In case of SPPS, all resins being known to the person skilled in the art and allowing the preparation of protected peptides can be applied. Here, resins are to be interpreted in a wide manner. Therefore, the term “resin” is to be understood to mean e.g. a solid support alone or a solid support directly linked to a linker, optionally with a handle in between. Typically, the solid support includes a linker to which the growing peptide is coupled during synthesis and which can be cleaved under desired conditions to release the peptide from the support. Suitable solid supports can have linkers that are electrophilically cleavable, such as trityl (trityl resins), chloromethyl (Merrifield resin), p-benzyloxybenzyl alcohol (Wang resin), 2-methoxy-4-alkoxybenzyl alcohol (SASRIN resin), benzhydrylamine (BHA resin), 4-methylbenzhydrylamine (MBNA resin), 4-(2,4-dimethoxyphenyl-Fmoc-aminomethyl)-phenoxy (Rink amide resin), 5-[(4-aminomethyl)-3,5-dimethoxyphenoxy]pentanoic acid (PAL amide resin), 9-Fmoc-aminoxanthen-3-yloxy (Sieber amide resin) or 4-(9-Fmoc-aminoxanthen-3-yloxy)butyryl (Xanthenyl, XAL, resin); nucleophilically cleavable; photocleavable; metal-assisted cleavable; cleavable under reductive conditions; or cleavable under oxidative conditions; as outlined in Guillier et al, Chem. Rev. 2000, 100, 2091-2157. Preferred linkers are electrophilically cleavable, especially by use of acid such as trifluoroacetic acid (TFA).

Particularly preferred linkers are cleavable under conditions that both the N-terminus and the side chains of the cleaved fragments II, V and VII, or of the cleaved peptide fragments (A), (B) and (CL), as defined further down in the text, are still substantially protected. Typically, cleavage is carried out by use of acid, such as diluted trifluoro-acetic acid. For the preparation of the N-terminally deprotected fragment IIIa/IIIb, or of the N-terminally deprotected peptide fragment (CR), as defined further down in the text, the N-terminally protected fragment-precursor can be cleaved from the resin under retention of the N-terminal and side chain protecting groups in a first step, followed by deprotection of the N-terminus in a second step. Alternatively, the N-terminally protected fragment-precursor can be deprotected at its N-terminus and then cleaved from the resin in the same step. Advantageously, the N-terminally deprotected fragment IIIa/IIIb, or the N-terminally deprotected peptide fragment (CR), as defined further down in the text, is obtained by deprotection at its N-terminus and then cleavage from the resin in the same step.

In one embodiment of the present invention, the resin for the SPPS preparation of the side-chain protected peptides of formula II, V and VII, or of the side chain protected peptide fragments (A), (B) and (CL), as defined further down in the text, is favorably chosen according to the criterion that the carboxylic acid is directly formed after cleavage from the resin. Suitable resins are for example trityl resins, like 2-chlorotrityl chloride resin (CTC resin), trityl chloride resin, 4-methyltrityl chloride resin or 4-methoxytrityl chloride resin; Merrifield resin, Wang resin and SASRIN resin. A particular suitable resin is the CTC resin.

In a further embodiment of the present invention, the resin for the SPPS preparation of the side-chain protected peptide of formula IIIa/IIIb, or of the side chain protected peptide fragment (CR), as defined further down in the text, is favorably chosen according to the criterion that the carboxamide is directly formed after cleavage from the resin, instead of laborious post-synthetic amidation of the carboxy group. Suitable resins are for example Sieber amide resin, BHA resin, MBHA resin, Rink amide resin, PAL amide resin and XAL resin. A particular suitable resin is the Sieber amide resin.

In another embodiment of the present invention, the resin for the SPPS preparation of the side-chain protected peptide of formula IIIa/IIIb, or of the side chain protected peptide fragment (CR), as defined further down in the text, is favorably chosen according to the criterion that the resin is suitable for attaching the first amino acid, in its amide form and N-terminally protected, via its side chain to the resin. This means a suitable resin is suitable for attaching the side chain of N-terminally protected serinamide, such as Fmoc-Ser-NH₂, for the preparation of the side-chain protected peptide of formula IIIa, or of the side chain protected peptide fragment (CRX2-Y1) with Y1 being 39, with the peptide fragment (CRX2-Y1) being defined further down in the text, and which is suitable for attaching the side chain an N-terminally protected lysinamide, such as Fmoc-Lys-NH₂, for the preparation of the side-chain protected peptide of formula IIIb, or of the side chain protected peptide fragment (CRX2-Y1) with Y1 being 44, with the peptide fragment (CRX2-Y1) being defined further down in the text. Suitable resins are for example trityl resins, like 2-chlorotrityl chloride resin (CTC resin), trityl chloride resin, 4-methyltrityl chloride resin or 4-methoxytrityl chloride resin. A particular suitable resin is the CTC resin. As the suitable resins are much cheaper than the resins by which the carboxamide is directly formed after cleavage (see embodiment above), use of such resins is advantageous, especially for production on commercial scale.

In another embodiment of the present invention, amidation, that is the preparation, of the side chain-protected peptide of formula IIIa is accomplished in solution phase by
(a) reacting a side chain-protected peptide of formula

(SEQ ID NO 12)
P4-³⁰Gly-Pro-Ser-Ser-Gly-³⁵Ala-Pro-Pro-Pro-OH
(IXa),

in the following abbreviated with P4-[30-38]-OH;
wherein P4 is an carbamate-type protecting group,
with a side chain-protected amino acid of formula

| | |
|---|---|
| H-³⁹Ser-NH₂ | (Xa), |

to produce a side chain-protected peptide of formula

(SEQ ID NO 4)
P4-³⁰Gly-Pro-Ser-Ser-Gly-³⁵Ala-Pro-Pro-Pro-Ser-NH₂
(XIa),

in the following abbreviated with P4-[30-39]-NH₂;
wherein P4 is as defined above, and
(b) removing the N-terminal protecting group P4 of the side chain-protected peptide of formula XIa to produce the side chain-protected peptide of formula

(SEQ ID NO 4)
H-³⁰Gly-Pro-Ser-Ser-Gly-³⁵Ala-Pro-Pro-Pro-Ser-NH₂
(IIIa);

which is the H-[30-39]-NH₂;
preferably, the side chain-protected peptide of formula IIIa is prepared in a precedent process.

Preferably, the carbamate protecting group P4 is Fmoc.

Suitably, the side chain-protected peptide of formula IXa is obtained by SPPS.

The coupling in step (a) is typically accomplished by the coupling mixture TOTU/HOBt/DIPEA in the preferred mixture ethyl acetate/DMF of the water-miscible solvent DMF and of the non water-miscible solvent ethyl acetate.

Preferably, after step (a) the produced side chain-protected peptide of formula XIa is not isolated before continuing with step (b).

In a preferred embodiment, N-terminal deprotection of step (b) is carried out by use of diethylamine in dichloromethane (DCM).

These reaction conditions of steps (a) and (b) for the preparation of peptide of formula (IIIa) from peptide of formula (IXa) and amino acid of formula (Xa) also apply for the below described preparation of peptide fragments (CR30-39), (CR31-39), (CR32-39), (CR26-39) and (CR27-39).

All SPPS-prepared peptide fragments, preferably the side chain-protected peptide fragments of formula II IIIa/IIIb, V, VII and IXa, or the side chain protected peptide fragments (CL), (CR), (B), (A), (CR30-38), (CR31-38), (CR32-38), (CR26-38) and (CR27-38) as defined further down in the text, can be prepared using known methods for SPPS assembly by the skilled person. Preferably, SPPS is accomplished following the Fmoc-protocol or the Boc-protocol and protection of the side chains with suitable side chain protecting groups. More preferably, SPPS is accomplished following the Fmoc-protocol with suitable side chain protecting groups. After attachment of the first amino acid to the resin, each single N-terminally and, optionally side chain-protected amino acid, or alternatively dipeptide, is assembled step-wise to the growing resin-bound peptide chain. Removal of the N-terminal protecting group is carried out under conditions depending on the nature of the protecting group. Typically, the N-terminus is deblocked by use of base, like piperidine, or mixtures of bases, like piperidine/1,8-diazabicyclo[5.4.0]-7-undecene (DBU), optionally in the presence of at least one scavenger like HOBt.

Coupling can be accomplished with in situ coupling reagents, and optional addition of scavengers and/or base. OXYMAPURE®, i.e. ethyl 2-cyano-2-hydroxyiminoacetate, has proved to be an effective scavenger as racemization is more suppressed compared to benzotriazole-based scavengers. In addition, it is less explosive than e.g. HOBt so that its handling is advantageous. Thus, OXYMAPURE® is a preferred scavenger.

Typically, DIC, DIC/HOBt, TCTU/CI-HOBt/DIPEA or DIC/OXYMAPURE® is employed. Alternatively, coupling may be carried out by reaction between a pre-activated N-terminally and, optionally side chain-protected amino acid and the N-terminally deprotected peptidyl resin. A pentafluorophenyl ester (OPfp) of an amino acid is typically employed as pre-activated amino acid.

In case the fragments IXa, IIIa/IIIb, to VIIIa/VIIIb bear a pseudoproline protecting group, the pseudoproline unit can be introduced by assembling the commercially available N-terminally protected pseudoproline dipeptide instead of the single N-terminally protected, conventionally side chain-protected serine or threonine. Suitable pseudoproline dipeptides are for example Fmoc-Ser(tBu)-Ser(ψ^{Me,Me}pro)-OH and Fmoc-Pro-Ser)ψ^{Me,Me}pro)-OH.

Another object of the present invention is to provide side chain-protected peptides which are useful as intermediates in the process of the invention. In particular, one of these peptides is a side chain-protected peptide selected from the group consisting of

a side chain-protected peptide of formula

(SEQ ID NO 8)
P2-Ser-Lys-Gln-Met-¹⁵Glu-Glu-Glu-Ala-Val-²⁰Arg-
Leu-OH
(V),

wherein P2 is an carbamate-type protecting group, preferably is Fmoc or Boc, most preferably is Fmoc,

a side chain-protected peptide of formula

(SEQ ID NO 3)
P1-Phe-Ile-Glu-²⁵Trp-Leu-Lys-Asn-Gly-OH
(II),

wherein P1 is an carbamate-type protecting group, preferably is Fmoc or Boc, most preferably is Fmoc,

a side chain-protected peptide of formula

(SEQ ID NO 12)
P4-³⁰Gly-Pro-Ser-Ser-Gly-³⁵Ala-Pro-Pro-Pro-OH
(IXa),

wherein P4 is an carbamate-type protecting group, preferably is Fmoc or Boc, most preferably is Fmoc, and

a side chain-protected peptide of formula

(SEQ ID NO 9)
P5-Ser-Lys-Gln-Met-¹⁵Glu-Glu-Glu-Ala-Val-²⁰Arg-
Leu-Phe-Ile-Glu-²⁵Trp-Leu-Lys--Asn-Gly-³⁰Gly-Pro-
Ser-Ser-Gly-³⁵Ala-Pro-Pro-Pro-Ser-NH₂
(XIIa),

respectively

(XIIb)
(SEQ ID NO 10)
P5-Ser-Lys-Gln-Met-¹⁵Glu-Glu-Glu-Ala-Val-²⁰Arg-
Leu-Phe-Ile-Glu-²⁵Trp-Leu-Lys--Asn-Gly-³⁰Gly-Pro-
Ser-Ser-Gly-³⁵Ala-Pro-Pro-Ser-Lys-⁴⁰Lys-Lys-Lys-
Lys-Lys-NH₂,

wherein P5 is an carbamate-type protecting group, preferably is Fmoc or Boc, most preferably is Fmoc; or hydrogen.

In a preferred embodiment, the side chain-protected peptide of formula V is

(SEQ ID NO 8)
Fmoc-Ser(tBu)-Lys(Boc)-Gln(Trt)-Met-¹⁵Glu(OtBu)-
Glu(OtBu)-Glu(OtBu)-Ala-Val--²⁰Arg(Pbf)-Leu-OH,

in the following abbreviated with Fmoc-[11-21]-OH—SCP;

which comprises the sequence of amino acid position 11-21 of exenatide, respectively of its analogue of formula Ib.

In another preferred embodiment, the side chain-protected peptide of formula II is

(SEQ ID NO 3)
Fmoc-Phe-Ile-Glu(OtBu)-²⁵Trp(Boc)-Leu-Lys(Boc)-
Asn(Trt)-Gly-OH,

in the following abbreviated with Fmoc-[22-29]-OH—SCP;

which comprises the sequence positions 22-29 of exenatide, respectively of its analogue of formula Ib.

In another preferred embodiment, the side chain-protected peptide of formula IXa is

(SEQ ID NO 12)
Fmoc-³⁰Gly-Pro-Ser(tBu)-Ser(tBu)-Gly-³⁵Ala-Pro-
Pro-Pro-OH,

in the following abbreviated with Fmoc-[30-38]-OH—SCP;

which comprises the sequence positions 30-38 of exenatide.

In another preferred embodiment, the side chain-protected peptide of formula XIIa is

(SEQ ID NO 9)
P5-Ser(tBu)-Lys(Boc)-Gln(Trt)-Met-¹⁵Glu(OtBu)-Glu
(OtBu)-Glu(OtBu)-Ala-Val--²⁰Arg(Pbf)-Leu-Phe-Ile-
Glu(OtBu)-²⁵Trp(Boc)-Leu-Lys(Boc)-Asn(Trt)-Gly-
³⁰Gly-Pro--Ser(tBu)-Ser(tBu)-Gly-³⁵Ala-Pro-Pro-
Pro-Ser(tBu)-NH₂,

wherein P5 is Fmoc or hydrogen,

which comprises the sequence positions 11-39 of exenatide.

In another preferred embodiment, the side chain-protected peptide of formula XIIb is

(SEQ ID NO 10)
P5-Ser(tBu)-Lys(Boc)-Gln(Trt)-Met-¹⁵Glu(OtBu)-Glu
(OtBu)-Glu(OtBu)-Ala-Val--²⁰Arg(Pbf)-Leu-Phe-Ile-
Glu(OtBu)-²⁵Trp(Boc)-Leu-Lys(Boc)-Asn(Trt)-Gly-
³⁰Gly-Pro--Ser(tBu)-Ser(tBu)-Gly-³⁵Ala-Pro-Pro-Ser
(tBu)-Lys(Boc)-⁴⁰Lys(Boc)-Lys(Boc)--Lys(Boc)-Lys
(Boc)-Lys(Boc)-NH₂,

wherein P5 is Fmoc or hydrogen.

which comprises the sequence positions 11-44 of the exenatide analogue of formula Ib.

In another aspect, the present invention relates to the use of a side chain-protected peptide selected from the group consisting of formula

(II)
(SEQ ID NO 3)
P1-Phe-Ile-Glu-²⁵Trp-Leu-Lys-Asn-Gly-OH,

wherein P1 is an carbamate-type protecting group, preferably is Fmoc or Boc, most preferably is Fmoc,

(IXa)
(SEQ ID NO 12)
P4-³⁰Gly-Pro-Ser-Ser-Gly-³⁵Ala-Pro-Pro-Pro-OH,

wherein P4 is an carbamate-type protecting group, preferably is Fmoc or Boc, most preferably is Fmoc,

(IIIa)
(SEQ ID NO 4)
H-³⁰Gly-Pro-Ser-Ser-Gly-³⁵Ala-Pro-Pro-Pro-Ser-NH₂,

respectively

(IIIb)
(SEQ ID NO 5)
H-³⁰Gly-Pro-Ser-Ser-Gly-³⁵Ala-Pro-Pro-Ser-Lys-
⁴⁰Lys-Lys-Lys-Lys-Lys-NH₂,
(V)
(SEQ ID NO 8)
P2-Ser-Lys-Gln-Met-¹⁵Glu-Glu-Glu-Ala-Val-²⁰Arg-
Leu-OH,

wherein P2 is an carbamate-type protecting group, preferably is Fmoc or Boc, most preferably is Fmoc,

(VII)
(SEQ ID NO 11)
P3-¹His-Gly-Glu-Gly-⁵Thr-Phe-Thr-Ser-Asp-¹⁰Leu-OH,

wherein P3 is an carbamate-type protecting group, preferably is Fmoc or Boc, most preferably is Boc,

(IVa)
(SEQ ID NO 6)
P1-Phe-Ile-Glu-²⁵Trp-Leu-Lys-Asn-Gly-³⁰Gly-Pro-
Ser-Ser-Gly-³⁵Ala-Pro-Pro-Pro--Ser-NH₂,

respectively

(IVb)
(SEQ ID NO 7)
P1-Phe-Ile-Glu-²⁵Trp-Leu-Lys-Asn-Gly-³⁰Gly-Pro-
Ser-Ser-Gly-³⁵Ala-Pro-Pro-Ser--Lys-⁴⁰Lys-Lys-Lys-
Lys-Lys-NH₂,

wherein P1 is an carbamate-type protecting group, preferably is Fmoc or Boc, most preferably is Fmoc; or hydrogen,

(VIa)
(SEQ ID NO 9)
P2-Ser-Lys-Gln-Met-¹⁵Glu-Glu-Glu-Ala-Val-²⁰Arg-
Leu-Phe-Ile-Glu-²⁵Trp-Leu-Lys--Asn-Gly-³⁰Gly-Pro-
Ser-Ser-Gly-³⁵Ala-Pro-Pro-Pro-Ser-NH₂,

respectively

(VIb)
(SEQ ID NO 10)
P2-Ser-Lys-Gln-Met-¹⁵Glu-Glu-Glu-Ala-Val-²⁰Arg-
Leu-Phe-Ile-Glu-²⁵Trp-Leu-Lys--Asn-Gly-³⁰Gly-Pro-
Ser-Ser-Gly-³⁵Ala-Pro-Pro-Ser-Lys-⁴⁰Lys-Lys-Lys-
Lys-Lys-NH₂,

wherein P2 is an carbamate-type protecting group, preferably is Fmoc or Boc, most preferably is Fmoc; or hydrogen, and

(VIIIa)
(SEQ ID NO 1)
P3-¹His-Gly-Glu-Gly-⁵Thr-Phe-Thr-Ser-Asp-¹⁰Leu-
Ser-Lys-Gln-Met-¹⁵Glu-Glu-Glu--Ala-Val-²⁰Arg-Leu-
Phe-Ile-Glu-²⁵Trp-Leu-Lys-Asn-Gly-³⁰Gly-Pro-Ser-
Ser-Gly-³⁵Ala--Pro-Pro-Pro-Ser-NH₂,

respectively

(VIIIb)
(SEQ ID NO 2)
P3-¹His-Gly-Glu-Gly-⁵Thr-Phe-Thr-Ser-Asp-¹⁰Leu-
Ser-Lys-Gln-Met-¹⁵Glu-Glu-Glu--Ala-Val-²⁰Arg-Leu-
Phe-Ile-Glu-²⁵Trp-Leu-Lys-Asn-Gly-³⁰Gly-Pro-Ser-
Ser-Gly-³⁵Ala--Pro-Pro-Ser-Lys-⁴⁰Lys-Lys-Lys-Lys-
Lys-NH₂,

wherein P3 is an carbamate-type protecting group, preferably is Fmoc or Boc, most preferably is Boc,

as intermediate in a synthesis of exenatide of formula

(Ia)
(SEQ ID NO 1)
H-¹His-Gly-Glu-Gly-⁵Thr-Phe-Thr-Ser-Asp-¹⁰Leu-Ser-
Lys-Gln-Met-¹⁵Glu-Glu-Glu--Ala-Val-²⁰Arg-Leu-Phe-
Ile-Glu-²⁵Trp-Leu-Lys-Asn-Gly-³⁰Gly-Pro-Ser-Ser-
Gly-³⁵Ala--Pro-Pro-Pro-Ser-NH₂,

respectively of the exenatide analogue of formula

(Ib)
(SEQ ID NO 2)
H-¹His-Gly-Glu-Gly-⁵Thr-Phe-Thr-Ser-Asp-¹⁰Leu-Ser-
Lys-Gln-Met-¹⁵Glu-Glu-Glu--Ala-Val-²⁰Arg-Leu-Phe-
Ile-Glu-²⁵Trp-Leu-Lys-Asn-Gly-³⁰Gly-Pro-Ser-Ser-
Gly-³⁵Ala--Pro-Pro-Ser-Lys-⁴⁰Lys-Lys-Lys-Lys-Lys-
NH₂.

The term “fragment” means “peptide fragment” and vice versa for the purpose of this invention, if not otherwise stated.

The nomenclature of amino acids or of peptides is based on Pure & Appl. Chem., Vo. 56, No. 5, pp. 595-624, 1984, “Nomenclature and symbolism for amino acids and peptides”, if not otherwise stated.

The term “#Xaa” signifies the amino acid Xaa at position # of SEQ ID NO 1, if not otherwise stated; e.g. ¹⁵Glu is the Glu at position 15 of SEQ ID NO 1.

The amino acids in positions 38 and 39 of SEQ ID NO 1 and SEQ ID NO 2 differ: In SEQ ID NO 1, it is ³⁸Pro³⁹Ser, in SEQ ID NO 2 it is ³⁸Ser³⁹Lys. In this application, any peptide fragment ending with position 38 or 39 is based in SEQ ID NO 1, any peptide fragment ending with position 44 is derived from SEQ ID NO 2, if not otherwise stated.

SPS means solution phase synthesis.

SPPS means solid phase peptide synthesis.

The possible embodiments for the reaction conditions of SPS and SPPS have been described above and apply as well to the various SPSs and SPPSs defined in the following text.

WO 2009/053315 A1 discloses on page 20 lines 8 to 13 and in FIG. 1 a synthesis scheme (10) for preparing exenatide from a first peptide fragment (12), a second peptide fragment (14) and a third peptide fragment (16). A fourth fragment (20) is prepared from the third fragment (16) by coupling with serine. Then the fourth fragment (20) is coupled with the second fragment (14) to provide the fifth fragment (22). Finally, the fifth fragment (22) is coupled with the first fragment (12) to obtain the insulinotropic peptide (11), which is the exenatide.

On page 3 line 25 to page 4 line 3, the WO'315 teaches, that
“A key challenge in the solid and solution phase synthesis of Exenatide relates to the sequence of three glutamic acid residues in the 15, 16 and 17 positions. Indeed, any peptide having at least two glutamic acid residues in sequence like this will tend to share this challenge. Specifically, it is difficult to chemically synthesize peptide fragments very far beyond such glutamic acid residues. Without wishing to be bound by theory, the repeating Glu sequence tends to yield a fragment portion that twists in the solid phase. This makes it relatively difficult to continue to build fragment size through the Glu chain effectively. In conventional practice, a fragment having a sequence of two or more repeating Glu residues might only be able to have 1 to 3 amino acids upstream (toward the C terminus) and/or downstream (toward the N-terminus) as a practical matter.”

And the WO'315 stresses the point on page 4 lines 3 and 4, that “The issue tends to be more severe downstream from the repeating Glu chain.”

As draw back resulting from this situation, the WO'315 points out that a 4 fragment strategy might be necessary, but sees even here disadvantages, see page 4 lines 7 to 18.

As a remedy, the WO'315 teaches on page 4 line 30 to page 5 line 3, that “It has been found that long peptide fragments incorporating these repeating Glu sequences can be readily synthesized when the fragments also incorporate one or more pseudoproline residues as a substitute for two corresponding amino acid residues.”

On page 5 line 32 to page 6 line 1, the WO'315 teaches, that “Without using at least one pseudoproline, at least four peptide fragments would be needed to apply a hybrid synthesis effectively.” and on page 6 lines 4 to 6, it again stresses the point, that “whereas only much shorter fragments including the Glu-Glu-Glu sequence can be synthesized in comparable yield and purity in the absence of using pseudoproline substitution(s).”

These suggested pseudoprolines are dipeptides comprising at least one amino acid selected from the group consisting of Ser and Thr, wherein the side chain is protected as a oxazolidine ring, as described in the WO'315 on page 23 line 12 to page 24 line 6.

On page 25 lines 9 to 15, the WO'315 discloses, that fragment (12) has at least one pseudoproline and being X^{j,k}Exenatide(1-m), Xi^{j,k} denoting the pseudoproline residue, and with m being 15 to 20 and marking the C-terminal amino acid of fragment (12) with respect to the sequence of exenatide.

The second peptide fragment (14) is disclosed on page 29 of the WO'315 being exenatide(n-q) wherein n is m+1 (wherein m is defined above with respect to the first fragment as being 15-20) and q is 25 to 30;

and the third peptide fragment (16) is disclosed on page 31 lines 1 to 5 of the WO'315, being exenatide(q+1-38).

Due to this technical prejudice of the WO'315, the skilled person would not consider a synthetic approach without the use of pseudoprolines.

There was a need for an efficient synthesis strategy for the preparation of exenatide. Surprisingly it has been found, that exenatide can be produced efficiently with a specific combination of solid and solution phase approach.

Subject of the invention is a method for the preparation of a peptide (1),

the peptide (1) being selected from the group consisting of peptide (2) and peptide (3),
the peptide (2) having the formula (Ia) as defined above;
the peptide (3) having the formula (Ib) as defined above;

characterized by preparing the peptide (1) with a three-fragment-strategy from peptide fragments (A), (B) and (C) by SPS,

the peptide fragment (B) being derived from peptide (1),

the peptide fragment (B) having as N-terminal amino acid the amino acid of position 11 of peptide (1); and

the peptide fragment (B) having as C-terminal amino acid the amino acid of position XB of peptide (1), with XB being 20, 21, 22, 23, 24, 25 or 26;

the peptide fragment (B) thereby having the sequence ¹¹Ser to ^{XB}Xaa of peptide (1);

the peptide fragment (B) bearing a N-terminal protecting group PGB of the carbamate-type;

the peptide fragment (B) being side-chain protected,

with the proviso, that peptide fragment (B) has no pseudoproline;

the peptide fragment (A) having the formula (VII), that is the P3-[1-10]-OH, as defined above;

the peptide fragment (C) being selected from the group consisting of peptide fragments (CX1-Y1),
the peptide fragments (CX1-Y1) being derived from peptide (1),
X1 is XB+1, with XB being as defined above, and X1 designating the N-terminal amino acid of peptide fragment (C), which is the amino acid of position X1 of peptide (1), and
Y1 is 39 or 44 and designates the C-terminal amino acid of peptide fragment (C), which is the amino acid 39 of peptide (2) or the amino acid 44 of peptide (3) respectively;

the peptide fragment (C) thereby having the sequence ^{X1}Xaa to ^{Y1}Xaa of peptide (1);

the peptide fragment (C) bearing no N-terminal protecting group;

the peptide fragment (C) being side-chain protected;

further characterized that

in a first step (c-ex) the peptide fragment (B) is coupled with the peptide fragment (C), resulting in a peptide fragment (D) bearing an N-terminal protecting group PGB;

and then

in a second step (d-ex), the N-terminal protecting group PGB of the peptide fragment (D) is removed;

and then

in a third step (e-ex), the peptide fragment (D) is coupled with the peptide fragment (A) resulting in peptide (1) bearing a protecting group P3,

and then

in a fourth step (f-ex), the N-terminal protecting group P3 is removed from peptide (1),

and in this step (f-ex) or afterwards,the side chain protecting groups are removed from peptide (1).

The step (c-ex) comprises the step (c) as defined above.

The step (d-ex) comprises the step (d) as defined above.

The step (e-ex) comprises the step (e) as defined above.

The step (f-ex) comprises the step (f) as defined above.

Preferably, none of the peptide fragments (A), (B) and (C) has a pseudoproline.

Preferably, pseudoproline is not used at all in any of the steps of the synthesis of peptide (1).

Preferably, the protecting group PGB is selected from the group consisting of fluoren-9-ylmethoxycarbonyl (Fmoc), tert-butoxycarbonyl (Boc) and allyloxycarbonyl (Alloc).

More preferably, PGB is Fmoc.

PGB comprises in specific embodiments P2 as defined above.

The peptide fragment (D) is a peptide fragment (D2) or a peptide fragment (D3),

the peptide fragment (D2) having the amino acid sequence (SEQ ID NO 9) and the formula (VIaa),

(VIaa)
(SEQ ID NO 9)
PGB-Ser-Lys-Gln-Met-¹⁵Glu-Glu-Glu-Ala-Val-²⁰Arg-
Leu-Phe-Ile-Glu-²⁵Trp-Leu--Lys-Asn-Gly-³⁰Gly-Pro-
Ser-Ser-Gly-³⁵Ala-Pro-Pro-Pro-Ser-NH₂,

and being abbreviated with PGB-[11-39]-NH₂ in the following, which comprises the P2-[11-39]-NH₂ as defined above, wherein PGB is P2;

the peptide fragment (D3) having the amino acid sequence (SEQ ID NO 10) and having the formula (VIba),

(VIba)
(SEQ ID NO 10)
PGB-Ser-Lys-Gln-Met-¹⁵Glu-Glu-Glu-Ala-Val-²⁰Arg-
Leu-Phe-Ile-Glu-²⁵Trp-Leu--Lys-Asn-Gly-³⁰Gly-Pro-
Ser-Ser-Gly-³⁵Ala-Pro-Pro-Ser-Lys-⁴⁰Lys-Lys-Lys-
Lys--Lys-NH₂,

and being abbreviated with PGB-[11-44]-NH₂ in the following, which comprises the P2-[11-44]-NH₂ as defined above, wherein PGB is P2;

with PGB being as defined above, also in all its preferred embodiments.

Further subject of the invention is the peptide fragment (B) as defined above.

Peptide fragment (B) comprises above mentioned peptide fragments of formula V.

Preferably, XB is 21, 25 or 26, more preferably 21.

Preferably, the peptide fragment (B) is selected from the group of peptide fragments (B1), (B2) and (B3),

the peptide fragment (B1) having the formula (B-XX);

(B-XX)
(SEQ ID NO 8)
PGB-Ser-Lys-Gln-Met-¹⁵Glu-Glu-Glu-Ala-Val-²⁰Arg-
Leu-OH

in following abbreviated with PGB-[11-21]-OH;

with PGB-[11-21]-OH comprising in specific embodiments the P2-[11-21]-OH as defined above;

the peptide fragment (B2) having the formula (B-XXI);

(B-XXI)
(SEQ ID NO 13)
PGB-Ser-Lys-Gln-Met-¹⁵Glu-Glu-Glu-Ala-Val-²⁰Arg-
Leu-Phe-Ile-Glu-²⁵Trp-Leu-OH

in following abbreviated with PGB-[11-26]-OH;

the peptide fragment (B3) having the formula (B-XXII);

(B-XXII)
(SEQ ID NO 14)
PGB-Ser-Lys-Gln-Met-¹⁵Glu-Glu-Glu-Ala-Val-²⁰Arg-
Leu-Phe-Ile-Glu-²⁵Trp-OH

in following abbreviated with PGB-[11-25]-OH.

More preferably, the peptide fragment (B) is selected from the group of peptide fragments (B1-SCP), (B2-SCP) and (B3-SCP);

the peptide fragment (B1-SCP) being Fmoc-[11-21]-OH—SCP as defined above;

the peptide fragment (B2-SCP) having the formula (B2-XXIII);

(B2-XXIII)
(SEQ ID NO 13)
Fmoc-Ser(tBu)-Lys(Boc)-Gln(Trt)-Met-¹⁵Glu(OtBu)-
Glu(OtBu)-Glu(OtBu)-Ala-Val--²⁰Arg(Pbf)-Leu-Phe-
Ile-Glu(OtBu)-²⁵Trp(Boc)-Leu-OH,

in following abbreviated with Fmoc-[11-26]-OH—SCP;

the peptide fragment (B3-SCP) having the formula (B3-XXIV);

(B3-XXIV)
(SEQ ID NO 14)
Fmoc-Ser(tBu)-Lys(Boc)-Gln(Trt)-Met-¹⁵Glu(OtBu)-
Glu(OtBu)-Glu(OtBu)-Ala-Val--²⁰Arg(Pbf)-Leu-Phe-
Ile-Glu(OtBu)-²⁵Trp(Boc)-OH,

in following abbreviated with Fmoc-[11-25]-OH—SCP.

Further subject of the invention is a method for the preparation of the peptide fragment (B), the peptide fragment (B) being as defined above, also in all its preferred embodiments, by SPPS or by SPS or by a combination thereof.

Preferably, the peptide fragment (B) is prepared by SPPS, the details of the SPPS being preferably as described above for the SPPS of fragments II, IIIa/IIIb, V and VII, more preferably V, also in all its preferred embodiments.

Further subject of the invention is the use of the peptide fragment (B), the peptide fragment (B) being as defined above, also in all its preferred embodiments, for the preparation of the peptide (1), the peptide (1) being as defined above, also in all its preferred embodiments.

Preferably, the peptide fragment (B) is used in SPS for the preparation of the peptide (1), preferably the SPS being as defined above, also with all its preferred embodiments.

Further subject of the invention is the peptide fragment (A).

Preferably, peptide fragment (A) is a peptide fragment (A-SCP)

the peptide fragment (A-SCP) having the formula (A-XX);

(A-XX)
(SEQ ID NO 11)
Boc-¹His(Trt)-Gly-Glu(OtBu)-Gly-⁵Thr(tBu)-Phe-Thr
(tBu)-Ser(tBu)-Asp(OtBu)-¹⁰Leu-OH

in following abbreviated with Boc-[1-10]-OH—SCP.

Further subject of the invention is a method for the preparation of the peptide fragment (A), the peptide fragment (A) being as defined above, also in all its preferred embodiments, by SPPS or by SPS or by a combination thereof.

Preferably, the peptide fragment (A) is prepared by SPPS, the details of the SPPS for the preparation of peptide fragment (A) being as described above for the SPPS of fragments II, IIIa/IIIb, V and VII, more preferably VII, also in all its preferred embodiments.

Further subject of the invention is the use of the peptide fragment (A), the peptide fragment (A) being as defined above, also in all its preferred embodiments, for the preparation of the peptide (1), the peptide (1) being as defined above, also in all its preferred embodiments.

Preferably, the peptide fragment (A) is used in SPS for the preparation of the peptide (1), preferably the SPS being as defined above, also with all its preferred embodiments.

Further subject of the invention is a peptide fragment (C) as defined above.

Further subject of the invention is a N-terminal protected peptide fragment (C), the protecting group being PC, with PC being a carbamate type protecting group, preferably Fmoc, Boc or Alloc, more preferably Fmoc or Boc, even more preferably Fmoc.

PC comprises in specific embodiments P1 as defined above; in other specific embodiments, PC comprises P4 as defined above.

In case of peptide (1) being peptide (2), peptide fragment (C) is preferably the peptide fragment (C22-39), (C26-39) or (C27-39), the peptide fragment (C22-39) having the amino acid sequence (SEQ ID NO 6) and being abbreviated with H-[22-39]-NH₂ in the following;

the peptide fragment (C26-39) having the amino acid sequence

(SEQ ID NO 15)
H-Leu-Lys-Asn-Gly-³⁰Gly-Pro-Ser-Ser-Gly-³⁵Ala-Pro-
Pro-Pro-Ser-NH²,

and being abbreviated with H-[26-39]-NH₂ in the following;

the peptide fragment (C27-39) having the amino acid sequence

(SEQ ID NO 16)
H-Lys-Asn-Gly-³⁰Gly-Pro-Ser-Ser-Gly-³⁵Ala-Pro-Pro-
Pro-Ser-NH₂,

and being abbreviated with H-[27-39]-NH₂ in the following.

The N-terminal protected peptide fragment (C) is preferably the N-terminal protected peptide fragment (C22-39), C(26-39) or (C27-39), with the N-terminal protecting group being PC, with PC being as defined above, also with all its preferred embodiments, the N-terminal protected peptide fragment (C22-39) having the amino acid sequence (SEQ ID NO 6),
and being abbreviated with PC-[22-39]-NH₂ in the following, which comprises the P1-[22-39]-NH₂ as defined above, wherein PC is P1;

the N-terminal protected peptide fragment (C26-39) having the amino acid sequence (SEQ ID NO 15),
and being abbreviated with PC-[26-39]-NH₂ in the following;

the N-terminal protected peptide fragment (C27-39) having the amino acid sequence (SEQ ID NO 16),
and being abbreviated with PC-[27-39]-NH₂ in the following.

In case of peptide (1) being peptide (3),

peptide fragment (C) is preferably the peptide fragment (C22-44), the peptide fragment (C22-44) having the amino acid sequence (SEQ ID NO 7) and being abbreviated with H-[22-44]-NH₂ in the following;

the N-terminal protected peptide fragment (C) is preferably the N-terminal protected peptide fragment (C22-44), the N-terminal protected peptide fragment (C22-44) being abbreviated with PC-[22-44]-NH₂ in the following, which comprises the P1-[22-44]-NH₂ as defined above, wherein PC is P1.

Further subject of the invention is a method for the preparation of the peptide fragment (C), wherein the peptide fragment (C) is prepared from a N-terminally protected peptide fragment (C),
which is N-terminally protected by a N-terminal protecting group PC,
and which is prepared by SPS, by SPPS, or by a combination of SPPS and SPS,
with PC being a carbamate type protecting group,

and subsequent removal of the N-terminal protecting group PC.

Preferably, the peptide fragment (C) is prepared by a step (a-ex), the step (a-ex) being a SPS coupling of a peptide fragment (CL) with a peptide fragment (CR);

wherein

the peptide fragment (CL) being selected from the group consisting of peptide fragments (CLX1-Y2),

which are derived from peptide (1),

wherein

X1 being as defined above, and

Y2 is 29, 30 or 31 and designates the C-terminal amino acid of peptide fragment (CL), which is the amino acid Y2 of peptide (1);

the peptide fragment (CL) thereby having the sequence ^{X1}Xaa to ^{Y2}Xaa of peptide (1);

the peptide fragment (CL) bearing PC, with PC being as defined above, also with all its preferred embodiments;

the peptide fragment (CL) being side-chain protected; and wherein

the peptide fragment (CR) being selected from the group consisting of peptide fragments (CRX2-Y1),

which are derived from peptide (1),

wherein

X2 is Y2+1 and designates the N-terminal amino acid of peptide fragment (CR), which is the amino acid of position X2 of peptide (1), and

Y1 being as defined above;

the peptide fragment (CR) thereby having the sequence ^{X2}Xaa to ^{Y1}Xaa of peptide (1);

the peptide fragment (CR) bearing no N-terminal protecting group;

the peptide fragment (CR) being side-chain protected;

this SPS coupling of peptide fragment (CL) with peptide fragment (CR) comprising the step (a) as defined above;

and subsequent removal of the N-terminal protecting group PC.

In the case, that Y1 is 44, then preferably XB is 21 and Y2 is 29.

Preferably, the peptide fragment (C22-39) is prepared by solution phase synthesis by coupling

a peptide fragment (CL22-29) with a peptide fragment (CR30-39),

a peptide fragment (CL22-30) with a peptide fragment (CR31-39), or

a peptide fragment (CL22-31) with a peptide fragment (CR32-39);

the peptide fragment (CL22-29) having the amino acid sequence (SEQ ID NO 3),

being abbreviated with PC-[22-29]-OH in the following, which comprises the P1-[22-29]-OH as defined above, wherein PC is P1;

the peptide fragment (CR30-39) being the H-[30-39]-NH₂ as defined above;

the peptide fragment (CL22-30) having the amino acid sequence

(SEQ ID NO 17)
PC-Phe-Ile-Glu-²⁵Trp-Leu-Lys-Asn-Gly-³⁰Gly-OH,

being abbreviated with PC-[22-30]-OH in the following;

the peptide fragment (CR31-39) having the amino acid sequence

(SEQ ID NO 18)
H-Pro-Ser-Ser-Gly-³⁵Ala-Pro-Pro-Pro-Ser-NH₂,

being abbreviated with H-[31-39]-NH₂ in the following;

the peptide fragment (CL22-31) having the amino acid sequence

(SEQ ID NO 19)
PC-Phe-Ile-Glu-²⁵Trp-Leu-Lys-Asn-Gly-³⁰Gly-Pro-OH,

being abbreviated with PC-[22-31]-OH in the following;

the peptide fragment (CR32-39) having the amino acid sequence

(SEQ ID NO 20)
H-Ser-Ser-Gly-³⁵Ala-Pro-Pro-Pro-Ser-NH₂,

being abbreviated with H-[32-39]-NH₂ in the following;

and

subsequent removal of the N-terminal protecting group PC.

Preferably, the peptide fragment (C22-44) is prepared by coupling

the peptide fragment (CL22-29) with a peptide fragment (CR30-44),

the peptide fragment (CL22-30) with a peptide fragment (CR31-44), or

the peptide fragment (CL22-31) with a peptide fragment (CR32-44);

the peptide fragment (CR30-44) being the H-[30-44]-NH₂ as defined above;

the peptide fragment (CR31-44) having the sequence

(SEQ ID NO 21)
H-Pro-Ser-Ser-Gly-³⁵Ala-Pro-Pro-Ser-Lys-⁴⁰Lys-
Lys-Lys-Lys-Lys-NH₂,

being abbreviated with H-[31-44]-NH₂ in the following;

the peptide fragment (CR32-44) having the sequence

(SEQ ID NO 22)
H-Ser-Ser-Gly-³⁵Ala-Pro-Pro-Ser-Lys-⁴⁰Lys-Lys-
Lys-Lys-Lys-NH₂,

being abbreviated with H-[32-44]-NH₂ in the following;

more preferably, the peptide fragment (C22-44) being produced by coupling the peptide fragment (CL22-29) with a fragment (CR30-44),

and

subsequent removal of the N-terminal protecting group PC.

Preferably, the peptide fragment (C26-39) and the peptide fragment (C27-39) are prepared by SPS;

more preferably, the peptide fragment (C26-39) is prepared by solution phase coupling of a peptide fragment (C26-38) with H-Ser-NH_{2;}

more preferably, the peptide fragment (C27-39) is prepared by solution phase coupling of a peptide fragment (C27-38) with H-Ser-NH_{2;}

the peptide fragment (C26-38) having the amino acid sequence

(SEQ ID NO 23)
PC-Leu-Lys-Asn-Gly-³⁰Gly-Pro-Ser-Ser-Gly-³⁵Ala-
Pro-Pro-Pro-OH,

being abbreviated with PC-[26-38]-OH in the following;

the peptide fragment (C27-38) having the amino acid sequence

(SEQ ID NO 24)
PC-Lys-Asn-Gly-³⁰Gly-Pro-Ser-Ser-Gly-³⁵Ala-Pro-
Pro-Pro-OH,

being abbreviated with PC-[27-38]-OH in the following;

and

subsequent removal of PC, with PC being as defined above, also in all its preferred embodiments;

this SPS preparation of peptide fragments (CR26-39) and (CR27-39) comprising the reaction conditions of steps (a) and (b), with the steps (a) and (b) being as described above for the preparation of peptide of formula (111a) starting from peptide of formula (IXa) and amino acid of (Xa), providing peptide of formula (XIa), which is step (a), and subsequent deprotecting in step (b) providing peptide of formula (IIIa); with PC being P4.

Preferably, the peptide fragments (C26-38) and (C27-38) are prepared by SPPS.

Further subject of the invention is the use of the peptide fragment (C), the peptide fragment (C) being as defined above, also in all its preferred embodiments, for the preparation of the peptide (1), the peptide (1) being as defined above, also in all its preferred embodiments.

Preferably, the peptide fragment (C) is used in SPS for the preparation of the peptide (1), preferably the SPS being as defined above, also with all its preferred embodiments.

Further subject of the invention is the peptide fragment (CL), the peptide fragment (CL) being as defined above, also in all its preferred embodiments.

Preferably, the peptide fragment (CL) is selected from the group consisting of peptide fragments (CL22-29), (CL22-30) and (CL22-31), with the peptide fragments (CL22-29), (CL22-30) and (CL22-31) being as defined above.

Further subject of the invention is a method for the preparation of the peptide fragment (CL), the peptide fragment (CL) being as defined above, also in all its preferred embodiments, by SPPS or by SPS or by a combination thereof.

Preferably, the peptide fragment (CL) is prepared by SPPS, the details of the SPPS for the preparation of peptide fragment (CL) being as described above for the SPPS of fragments II, IIIa/IIIb, V and VII.

Further subject of the invention is the use of the peptide fragment (CL), the peptide fragment (CL) being as defined above, also in all its preferred embodiments, for the preparation of the peptide (C), the peptide (CL) being as defined above, also in all its preferred embodiments.

Preferably, the peptide fragment (CL) is used in SPS for the preparation of the peptide (C).

Further subject of the invention is the peptide fragment (CR) as defined above, also with all its preferred embodiments.

Further subject of the invention is a N-terminal protected peptide fragment (CR), the protecting group being PC, with PC being as defined above, also in all its preferred embodiments.

Further subject of the invention is a method for the preparation of the peptide fragment (CR), wherein the peptide fragment (CR) is prepared from a N-terminally protected peptide fragment (CR),
which is N-terminally protected by a N-terminal protecting group PC, with PC being as defined above, also with all its preferred embodiments,
and which is prepared by SPS, by SPPS, or by a combination of SPPS and SPS,

and subsequent removal of the N-terminal protecting group PC.

Preferably, the peptide fragments (CR30-39), (CR31-39) and (CR32-39) are prepared by SPS coupling;

more preferably, the peptide fragment (CR30-39) is prepared by solution phase coupling of a peptide fragment (CR30-38) with H-Ser-NH₂, which produces the P4-[30-39]-NH₂ as defined above, when PC is P4;

more preferably, the peptide fragment (CR31-39) is prepared by solution phase coupling of a peptide fragment (CR31-38) with H-Ser-NH₂;

more preferably, the peptide fragment (CR32-39) is prepared by solution phase coupling of a peptide fragment (CR32-38) with H-Ser-NH₂;

the peptide fragment (CR30-38)

(SEQ ID NO 12)
PC-³⁰Gly-Pro-Ser-Ser-Gly-³⁵Ala-Pro-Pro-Pro-OH,

being abbreviated with PC-[30-38]-OH in the following, which comprises the P4-[30-3]-OH as defined above, when PC is P4;

the peptide fragment (CR31-38)

(SEQ ID NO 25)
PC-Pro-Ser-Ser-Gly-³⁵Ala-Pro-Pro-Pro-OH,

being abbreviated with PC-[31-38]-OH in the following;

the peptide fragment (CR32-38)

| | |
|---|---|
| PC-Ser-Ser-Gly-³⁵Ala-Pro-Pro-Pro-OH, | (SEQ ID NO 26) |

being abbreviated with PC-[32-38]-OH in the following;

and

subsequent removal of PC;

this SPS preparation of peptide fragment (CR) comprising steps (a) and (b), with the steps (a) and (b) being as described above for the preparation of peptide of formula (IIIa) starting from peptide formula (IXa) and amino acid of (Xa), providing peptide of formula (XIa), which is step (a), and subsequent deprotecting in step (b) providing peptide of formula (IIIa), with PC being P4.

Preferably, the peptide fragments (CR30-38), (CR31-38), (CR32-38), (CR30-44), (CR31-44) and (CR32-44) are prepared by SPPS, and in case of the peptide fragments (CR30-44), CR(31-44) and (CR32-44) with subsequent removal of PC.

Preferably, the N-terminal protected peptide fragment D is prepared by coupling the N-terminal protected peptide fragment B1 with the peptide fragment (C22-39) or (C22-44);

the N-terminal protected peptide fragment B2 with a peptide fragment (C27-39);

the N-terminal protected peptide fragment B3 with a peptide fragment (C26-39).

The choice of the peptide fragment (C) or (CR) respectively, determines, whether peptide (2) or (3) is being prepared.

Further subject of the invention is a peptide fragment (D) as defined above, also with all its preferred embodiments.

Further subject of the invention is the use of the peptide fragment (D), the peptide fragment (D) being as defined above, also in all its preferred embodiments, for the preparation of the peptide (1), the peptide (1) being as defined above, also in all its preferred embodiments.

Preferably, the peptide fragment (D) is used in SPS for the preparation of the peptide (1), preferably the SPS being as defined above, also with all its preferred embodiments.

Against the teaching of the WO'315 and unexpectedly, it was possible to produce a peptide fragment (B), having as N-terminal amino acid the amino acid of position 11 of peptide (1), and having as C-terminal amino acid the amino acid of position XB of peptide (1), with XB being 20, 21, 22, 23, 24, 25 or 26; thereby comprising the three consecutive Glu residues -¹⁵Glu-¹⁶Glu-¹⁷Glu- of exenatide and downstream thereof the adjoining 4 amino acid residues -¹¹Ser-¹²Lys_{—}¹³Gln-¹⁴Met- of exenatide.

Due to the technical prejudice of the WO'315, the skilled person would not have considered a synthetic approach without the use of pseudoprolines.

By the specific fragment scission point between the position 10 and 11 of exenatide, it was possible to overcome the technical prejudice mentioned in the WO'315: It was possible to synthesize a peptide fragment with 3 consecutive Glu residues, but simultaneously having additional 4 amino acids, i.e. more then the 3 amino acids according to the technical prejudice, downstream, i.e. towards the N-terminus.

By this specific selection of the scission point, it was no longer necessary to use one or more pseudoprolines downstream of the three consecutive Glu residues, thereby simplifying the synthesis of exenatide by omitting the necessity of using separately prepared and purified dipeptides.

### Examples

The following non-limiting examples will illustrate representative embodiments of the invention in detail.

Abbreviations:
AcOH=acetic acid
Cl-HOBt=6-chloro-1-hydroxybenzotriazole
CTC=2-chlorotrityl chloride
DBU=1,8-diazabicyclo[5.4.0]-7-undecene
DCM=dichloromethane
DIC=diisopropylcarbodiimide
DIPEA=N,N-diisopropylethylamine
DIPE=diisopropyl ether
DMF=N,N-dimethylformamide
DMS=dimethylsulfide
EDT=ethanedithiol
eq=equivalent
HOBt hydrate=1-hydroxybenzotriazole hydrate
NMP=1-methyl-2-pyrrolidone
PyBOP=benzotriazol-1-yloxy-tris(pyrrolidino)phosphonium hexafluorophosphate
TBTU=O-(benzotriazol-1-yl)-1,1,3,3-tetramethyluronium tetrafluoroborate
TCTU=O-(6-chlorobenzotriazol-1-yl)-1,1,3,3-tetramethyluronium tetrafluoroborate TFA=trifluoroacetic acid
TIS=triisopropylsilane
TOTU=O-[cyano(ethoxycarbonyl)methylenamino]-1,1,3,3-tetramethyluronium tetrafluoroborate
   ACN acetonitrile
   Boc tert-butoxycarbonyl
   COMU 1-[(1-(cyano-2-ethoxy-2-oxo-ethylideneaminooxy)-dimethylaminomorpholinomethylene)]methanaminium hexafluorophosphate
   CTC resin 2-chlorotrityl chloride resin (100-200 mesh), particle size: 95-200 micrometer and loading 1.57 mmol/g resin the loading of the resin (mmol/g) means the mmol of reactive sites per g of 2-chlorotrityl chloride resin
   DIPCDI N,N-diisopropylcarbodiimide
   eq equivalent(s) eq refers to the mol-equivalents, with regard to the reactive sites of the resin if not mentioned otherwise
   Fmoc 9-fluorenylmethyloxycarbonyl
   h hour(s)
   HBTU O-benzotriazole-N,N,N′,N′-tetramethyl-uronium-hexafluoro-phosphate
   HOBt hydroxybenzotriazole
   HPLC high pressure liquid chromatography
   Milli-Q water purified and deionised to a high degree (water purification system manufactured by Millipore)
   min minutes(s)
   Pbf 2,2,4,6,7-pentamethyldihydrobenzofurane-5-sulfonyl
   Psi pseudoproline
   PsiSer pseudoproline derived from Ser
   PsiThr pseudoproline derived from Thr
   tBu tent-butyl
   Trt trityl
   UV ultraviolet

Methods

UV Quantification of Fmoc Removal

Quantification of the loading of the CTC resin was carried out by UV measurement after

Fmoc removal of the first amino acid coupled onto the resin.

The following apparatus are used in every solid phase synthesis examples as described in the procedure below: UV-visible recording spectrophotometer.

The procedure is described as follows:

Step1. Collection of the solution from the removal of the Fmoc group of the first amino acid coupled onto the CTC resin:
In a glass flask, with controlled volume, (100 ml per 3 g of resin and 250 ml per 6 g of resin) was collected the solution resulted from the removal of the Fmoc group protocol (by using a solution of piperidine in DMF (20% piperidine; 1 time 1 min; 2 times 5 min; 30 ml each); four to five cycles (four cycles in the case of a 3 g resin and five cycles in case of a 6 g resin) were carried out and the bed was drained and thoroughly washed with DMF).
The glass flask, with controlled volume, was filled until the total volume with DMF.

Step2. A dilution of the solution collected in step1 was prepared in DMF:
Two consecutive dilutions with 1 ml of concentrated solution, diluted with DMF was collected in a glass flask, with controlled volume, 10 ml. DMF was added until the total volume of 10 ml glass flask. The last diluted solution is the sample which is going to be measured.
Those dilutions are going to be prepared three times to have representative absorbance values of sample.

Step3. The solution from the second flask was measured in the UV spectrophotometer:
In a 1 cm length of quartz cell was added DMF and measured the absorbance at the wavelength of 290 nm (wavelength of maximum absorbance of dibenzofulvene), to obtain the UV zero.
The same cell was washed twice with the diluted solution and filled with the same solution. The absorbance of samples was measured at the wavelength of 290 nm.
The absorbance value is processed following that formulation and the final result is the loading of resin.

$$\frac{[ \begin{matrix}The absorbance measured \times \\ length of quartz cell \times \\ 100 or 250 \times 10 \times 10\end{matrix} ]}{[ 5800 \times grams of resin ]}$$

Length of quartz cell: 1 (cm)
100 or 250: the first glass flask, with controlled volume (ml)
10: volume of flask of dilutions (intermediate and last dilution) (ml)
5800: molar extinction coefficient of dibenzofulvene at 290 nm.
Grams of resin: 3 or 6 (g)

Determination of the Peptide Purity

The chromatographic analysis of the peptide fragments was performed by reverse phase chromatography.

The equipments used in each example are described in the procedure below:

Quaternary pump HPLC system, UV photodiode array detector.

The following reagents used in each example are described in the procedure below:

HPLC grade ACN, Milli-Q H₂O and TFA.

The procedure is described as follows:

Step1. The Mobile Phases A and B were made as follows:
Mobil Phase A was made by combining 901.1 g H₂O and 0.675 g TFA per 1 liter of mobile phase A (i.e., 999.5 ml H2O, 0.450 ml TFA)
Mobil Phase B was made by combining 760 g ACN, 0.540 g TFA per 1 liter of mobile phase B (i.e., 999.6 ml ACN, 0.360 ml TFA)

Step2. Install the column and set the following operating parameters:
Chromatography Conditions:
Column: SunFire C18, 3.5 micrometer, 4.6×100 mm
Oven: ambient
Flow rate: 1.0 ml/min
Detector wavelength:220 nm
Run time: 8 minutes
The sample is filtered through a 5 micrometer hydrophobic PTFE filter prior to the loading of the sample into the column.
Prior to the loading of the sample, the column is conditioned at initial conditions until a stable baseline is obtained: 3 minutes are used to equilibrate and to wash the column.

Step3. Measure of the area of all chromatography peaks related with the products from the synthesis. The areas proportion is directly related with the percentage of purity of the expected products.

### Example 1

### General Procedure for the Solid Phase Synthesis of all Fragments

1. Loading of the (N- and side chain-) protected C-terminal amino acid “n” of the fragment onto the resin.
2. Capping of unreacted active sites of the loaded resin.
3. N-Deprotection of currently N-terminal amino acid.
4. Chain elongation by coupling with the (N- and side chain-) protected amino acid “n-1”.
5. N-Deprotection of currently N-terminal amino acid.
6. Chain elongation by coupling with the (N- and side chain-) protected amino acid “n-2”,
7. and so on until completion of N- and side chain-protected fragment.
8. Cleavage of the N- and side chain-protected fragment from the resin.
9. Isolation of the N- and side chain-protected fragment.
10. Drying of the N- and side chain-protected fragment.

The (N- and side chain-) protected amino acids as well as the solid phase resins were available from Bachem, Iris Biotech, Novabiochem, Senn Chemicals, Genzyme or CBL Patras.

### Example 2

### Solid Phase Synthesis of Boc-¹His(Trt)-Gly-Glu(OtBu)-Gly-⁵Thr(tBu)-Phe-Thr(tBu)-Ser(tBu)-Asp(OtBu)-¹⁰Leu-OH (SEQ ID NO 11)

The synthesis was carried out in a 250 L solid phase reactor. Fmoc-Leu-OH (4.3 kg, 1.1 eq) was loaded onto CTC resin (15.8 kg, 1.0-1.6 mol/kg) in presence of DIPEA (5.1 L, 2.4 eq relative to the amino acid) in a mixture of DCM/DMF (2:3 v/v, 5-6 volumes). The reaction was monitored by HPLC. After completion, the bed was drained. The unreacted active positions on the resin were then capped by reaction with excess of methanol in the presence of DIPEA in DMF (DMF/methanol/DIPEA, 17:2:1 v/v/v, 7 volumes). Two capping cycles were carried out. The bed was drained, and thoroughly washed with DMF and NMP.

Removal of the Fmoc group was accomplished at 20-30° C. by using a solution of piperidine/DBU in DMF or in NMP (5% piperidine, 1% DBU, 7-8 volumes); two to three cycles were carried out. The bed was drained, and the H-Leu-CTC resin obtained was washed with DMF or NMP to remove residual piperidine.

A solution of Fmoc-Asp(OtBu)-OPfp (13.7 kg, 2.0 eq) in DMF (4-5 volumes) was added, and the coupling accomplished at 0° C. in the presence of DIPEA (pH=7-8). Its completeness was determined by the ninhydrin test. After complete coupling, the obtained Fmoc-Asp(OtBu)-Leu-CTC resin was drained and thoroughly washed with DMF.

The Fmoc group was removed as described above, the resin then treated at its free N-terminus with HOBt hydrate (2%) in DMF or NMP (7-8 volumes), and finally washed with DMF or NMP and drained.

HOBt hydrate (1.3 kg, 0.5-0-6 eq relative to the amino-acid) and Fmoc-Ser(tBu)-OH (6.3 kg, 1.5 eq), which was the subsequent amino acid in the sequence, were dissolved in DMF or NMP (4-5 volumes), and the amino acid solution transferred to the H-Asp(OtBu)-Leu-CTC resin. The mixture was reacted with DIC (5.2 L, 2.0 eq relative to the amino acid) at 20-30° C. The completeness of coupling was monitored by the ninhydrin or the chloranil test. After complete coupling, the peptidyl resin was drained and thoroughly washed with DMF or NMP.

The elongation cycle (Fmoc deprotection, HOBt hydrate treatment and amino acid coupling) was repeated for subsequent assembly of the peptide fragment using 1.5-3.0 eq each of Fmoc-Thr(tBu)-OH, Fmoc-Phe-OH, Fmoc-Thr(tBu)-OH, Fmoc-Gly-OH, Fmoc-Glu(OtBu)-OH, Fmoc-Gly-OH and Boc-His(Trt)-OH. In contrast to the coupling conditions as described above, no HOBt hydrate treatment was performed prior to the last coupling, which was carried out twice with PyBOP (8.6 kg, 1 eq relative to the amino acid) in NMP (4-5 volumes) in the presence of collidine (2.2 L, 1 eq relative to the amino acid) at 0° C.

After the last elongation cycle, the protected peptide was washed with DCM and cleaved from the resin by adding 3.0-0.5% TFA in DCM (6-13 volumes). Three cycles were carried out. The completion of cleavage was monitored by HPLC. After filtration, the peptidyl solutions were treated with DMF or NMP and neutralized with DIPEA. The combined solutions were concentrated by partial evaporation. The protected peptide precipitated after treatment with 0.1% potassium hydrogensulfate solution in water and was filtered off. The product was thoroughly washed with water and DIPE, and dried under reduced pressure affording 18.3 kg of Boc-¹His(Trt)-Gly-Glu(OtBu)-Gly-⁵Thr(tBu)-Phe-Thr(tBu)-Ser(tBu)-Asp(OtBu)-¹⁰Leu-OH (SEQ ID NO 11) as a white to beige powder with 95% purity and 99% recovery yield (based on a target batch size of 11.0 mol).

### Example 3

### Solid Phase Synthesis of Fmoc-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-OH (SEQ ID NO 8)

The synthesis was performed in a similar way as described in Example 2, using 12.9 kg (1.0-1.6 mol/kg) of CTC resin to which the C-terminal amino acid Fmoc-Leu-OH (3.3 kg, 1.1 eq) of the fragment was attached. The chain elongation was performed with 1.5-2.0 eq each of Fmoc-Arg(Pbf)-OH, Fmoc-Val-OH, Fmoc-Ala-OH, Fmoc-Glu(OtBu)-OH, Fmoc-Met-OH, Fmoc-Gln(Trt)-OH, Fmoc-Lys(Boc)-OH and Fmoc-Ser(tBu)-OH and using DIC/HOBt hydrate activation (2 eq relative to the amino acid/0.5 eq relative to the amino acid) except for Fmoc-Arg(Pbf)-OH coupling which was accomplished with TOTU/HOBt hydrate activation (1 eq relative to the amino acid/1 eq relative to the amino acid) in the presence of DIPEA (pH=7-8). Compared to Example 2, each Fmoc group was deprotected with piperidine in NMP (25% piperidine).

Yield: 20.5 kg of Fmoc-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-OH (SEQ ID NO 8) as a white to beige powder with 94% purity and 104% recovery yield (based on a target batch size of 8.4 mol).

### Example 4

### Solid Phase Synthesis of Fmoc-Phe-Ile-Glu(OtBu)-²⁵Trp(Boc)-Leu-Lys(Boc)-Asn(Trt)-Gly-OH (SEQ ID NO 3)

The synthesis was performed in a similar way as described in Example 2, using 14.2 kg (1.0-1.6 mol/kg) of CTC resin to which the C-terminal amino acid Fmoc-Gly-OH (5.6 kg, 1.1 eq) of the fragment was attached. The chain elongation was performed with 1.5-2.0 eq each of Fmoc-Asn(Trt)-OPfp, Fmoc-Lys(Boc)-OH, Fmoc-Leu-OH, Fmoc-Trp(Boc)-OH, Fmoc-Glu(OtBu)-OH, Fmoc-Ile-OH and Fmoc-Phe-OH.

Compared to Example 2, each Fmoc group was deprotected with piperidine in DMF (20% piperidine).

Yield: 27.4 kg of Fmoc-Phe-Ile-Glu(OtBu)-²⁵Trp(Boc)-Leu-Lys(Boc)-Asn(Trt)-Gly-OH (SEQ ID NO 3) as a white to beige powder with 95% purity and 93% recovery yield (based on a target batch size of 17 mol).

### Example 5

### Mixed Synthesis of Fmoc-³⁰Gly-Pro-Ser(tBu)-Ser(tBu)-Gly-³⁵Ala-Pro-Pro-Pro-Ser(tBu)-NH₂ (SEQ ID NO 4)

### Example 5a

### Solid Phase Synthesis of Fmoc-³⁰Gly-Pro-Ser(tBu)-Ser(tBu)-Gly-³⁵Ala-Pro-Pro-Pro-OH (SEQ ID NO 12)

The synthesis was performed in a similar way as described in Example 2, using 19.0 kg of CTC resin (1.0-1.6 mol/kg) to which the C-terminal dipeptide Fmoc-Pro-Pro-OH (9.6 kg, 1.1 eq) of the fragment was attached. The chain elongation was performed with 1.5-2.0 eq each of Fmoc-Pro-OH, Fmoc-Ala-OH, Fmoc-Gly-OH and Fmoc-Ser(tBu)-OH. Compared to Example 2, each Fmoc group was deprotected with piperidine and HOBt hydrate in DMF (25% piperidine, 2.5% HOBt hydrate). Moreover five cleavages cycles were carried out. After the partial evaporation, the protected peptide was directly precipitated and washed with DIPE.

Yield: 23.2 kg of Fmoc-³⁰Gly-Pro-Ser(tBu)-Ser(tBu)-Gly-³⁵Ala-Pro-Pro-Pro-OH (SEQ ID NO 12) as a white to beige powder with 97% purity and 106% recovery yield (based on a target batch size of 19.9 mol).

### Example 5b

### Solution Phase Synthesis of Fmoc-³⁰Gly-Pro-Ser(tBu)-Ser(tBu)-Gly-³⁵Ala-Pro-Pro-Pro-Ser(tBu)-NH₂ (SEQ ID NO 4)

H-Ser(tBu)-NH₂ (4.39 kg, 1 eq) and HOBt (3.73 kg, 1 eq) were added to a solution of the Fmoc-³⁰Gly-Pro-Ser(tBu)-Ser(tBu)-Gly-³⁵Ala-Pro-Pro-Pro-OH (SEQ ID NO 12) (28.70 kg, 1 eq; as obtained in Example 5a) in a mixture of ethyl acetate/DMF (10:1.5 v/v, 331 L). TOTU (8.56 kg, 1 eq) was added to the reaction mixture at 0° C. The pH was adjusted to 6.5-7 with DIPEA and the reaction was allowed to undergo to completion at 0° C. (monitored by HPLC and TLC). The reaction mixture was washed with a mixture of 1% aqueous potassium hydrogensulfate and 25% aqueous sodium chloride. The organic solution was evaporated under reduced pressure and dried by azeotropic distillation with portions of ethyl acetate. The residue was diluted with ethyl acetate, and the peptide was precipitated in DIPE. After filtration, the solid was washed with DIPE and dried under reduced pressure to yield 29.88 kg (92% recovery yield) of Fmoc-³⁰Gly-Pro-Ser(tBu)-Ser(tBu)-Gly-³⁵Ala-Pro-Pro-Pro-Ser(tBu)-NH₂ (SEQ ID NO 4) as a white powder with a purity of 97%.

### Example 6

### Mixed, Respectively Solid Phase Synthesis of H-³⁰Gly-Pro-Ser(tBu)-Ser(tBu)-Gly-³⁵Ala-Pro-Pro-Pro-Ser(tBu)-NH₂ (SEQ ID NO 4)

### Example 6a

### Mixed Synthesis of H-[30-39]-NH₂ (SEQ ID NO 4) [2 steps: First to Fmoc-[30-39]-NH₂ (SEQ ID NO 4) on Solid Phase, Second to H-[30-39]-NH₂(SEQ ID NO 4) in Solution Phase]

The synthesis was performed in a similar way as described in Examples 2 and 5a, using 50 g of Sieber amide resin (0.6 mol/kg) to which the C-terminal amino acid Fmoc-Ser(tBu)-OH (1.5 eq) of the fragment was attached after Fmoc deprotection.

The chain elongation was performed with 1.5 eq each of Fmoc-Ser(tBu)-OH, Fmoc-Pro-Pro-OH, Fmoc-Pro-OH, Fmoc-Ala-OH, Fmoc-Gly-OH and Fmoc-Ser(tBu)-OH as described in Example 2. In difference to Example 2, the coupling reactions were carried out with TCTU (0.95 eq relative to the amino acid)/CI-HOBt (1.0 eq relative to the amino acid)/(1.0 eq relative to the amino acid) in the presence of DIPEA (1.5 eq relative to the amino acid) at 20° C. in NMP/DCM (7:3, v/v). Also in difference to Example 2, after the last elongation cycle, one part (20%) of the protected peptidyl resin Fmoc-[30-39]-Sieber amide resin (SEQ ID NO 4), was washed with DCM and cleaved from the resin by adding 3-5% TFA in DCM (6-1 volumes) (another part of the protected peptidyl resin was used in Example 6b). Eight to twelve cycles were carried out. After filtration, part of the peptidyl solutions was neutralized with pyridine. The combined solutions were concentrated by partial evaporation, and the crystallized salt was removed by filtration. The protected peptide precipitated after treatment with DIPE and was filtered off. The product was thoroughly washed with DIPE, and dried under reduced pressure affording 4.6 g of Fmoc-³⁰Gly-Pro-Ser(tBu)-Ser(tBu)-Gly-³⁵Ala-Pro-Pro-Pro-Ser(tBu)-NH₂ (SEQ ID NO 4) as a solid with 78% purity and 60% recovery yield (based on a target batch size of 6 mmol).

In a following step, Fmoc-³⁰Gly-Pro-Ser(tBu)-Ser(tBu)-Gly-³⁵Ala-Pro-Pro-Pro-Ser(tBu)-NH₂ (SEQ ID NO 4) can be N-terminally deprotected as described in Example 7, to obtain H-³⁰Gly-Pro-Ser(tBu)-Ser(tBu)-Gly-³⁵Ala-Pro-Pro-Pro-Ser(tBu)-NH₂ (SEQ ID NO 4).

### Example 6b

### Solid Phase Synthesis of H-[30-39]-NH₂ (SEQ ID NO 4) (One Step)

Another part (13%) of the protected peptidyl resin Fmoc-[30-39]-Sieber amide resin (SEQ ID NO 4) (as obtained in Example 6a) was subjected to a mixture of piperidine and HOBt hydrate in DMF under conditions as described in Example 5a to form H-[30-39]-Sieber amide resin (SEQ ID NO 4). After washing with DCM, the peptide was cleaved from the resin with TFA in DCM under conditions as described in Example 6a.

The work-up procedure was performed similar to Example 6a.

Yield: 3.3 g of H-³⁰Gly-Pro-Ser(tBu)-Ser(tBu)-Gly-³⁵Ala-Pro-Pro-Pro-Ser(tBu)-NH₂ (SEQ ID NO 4) as a solid with 93% purity and 60% recovery yield (based on a target batch size of 4 mmol).

### Example 7

### Solution Phase Synthesis of H-³⁰Gly-Pro-Ser(tBu)-Ser(tBu)-Gly-³⁵Ala-Pro-Pro-Pro-Ser(tBu)-NH₂ (SEQ ID NO 4)

Diethylamine (11.23 L, 4.5 eq) was slowly added at 13° C. to 20° C. to a solution of Fmoc-³⁰Gly-Pro-Ser(tBu)-Ser(tBu)-Gly-³⁵Ala-Pro-Pro-Pro-Ser(tBu)-NH₂ (SEQ ID NO 4) (29.74 kg, 1 eq; as obtained in Example 5b) in DCM (59 L) and the reaction was allowed to go to completion at 20° C. (monitored by HPLC). The reaction mixture was evaporated under reduced pressure. Residual diethylamine was removed by azeotropic distillation with portions of toluene. The residue was diluted with DCM, and the peptide was precipitated with DIPE. After filtration, the final solid was washed with DIPE and dried under reduced pressure to yield 24.01 kg (99% recovery yield) of H-³⁰Gly-Pro-Ser(tBu)-Ser(tBu)-Gly-³⁵Ala-Pro-Pro-Pro-Ser(tBu)-NH₂ (SEQ ID NO 4) as a white powder with a purity of 91%.

### Example 8

### Solution Phase Synthesis of Fmoc-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 6)

TBTU (5.62 kg, 1 eq) was added at −5° C. to a mixture of Fmoc-Phe-Ile-Glu(OtBu)-²⁵Trp(Boc)-Leu-Lys(Boc)-Asn(Trt)-Gly-OH (SEQ ID NO 3) (30.20 kg, 1 eq; as obtained in Example 4) and HOBt (2.50 kg, 1 eq) in NMP (136 L). The pH was adjusted to 6.5-7 with DIPEA and the temperature was allowed to rise up to 0° C. After pre-activation of the Fmoc-[22-29]-OH (SEQ ID NO 3), a solution of H-³⁰Gly-Pro-Ser(tBu)-Ser(tBu)-Gly-³⁵Ala-Pro-Pro-Pro-Ser(tBu)-NH₂ (SEQ ID NO 4) (21.94 kg, 1 eq; as obtained in Example 7) in NMP (91 L) was added at 0° C. and the reaction was allowed to go to completion at pH 6.5-7 and 0° C. (completion monitored by HPLC). The reaction mixture was precipitated in water, and the precipitate was filtered off. Then, the precipitate was successively washed with water and DIPE and dried under reduced pressure to yield 48.13 kg (100% recovery yield) of Fmoc-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 6) as a white powder with a purity of 89%.

### Example 9

### Solution Phase Synthesis of H-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-N H₂ (SEQ ID NO 6)

Diethylamine (7.33 L, 4 eq) was slowly added at 20° C. to 25° C. to a solution of Fmoc-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 6) (47.87 kg, 1 eq; as obtained in Example 8) in a mixture of DCM/DMF (2.3:2.5, v/v, 230 L). The reaction was allowed to go to completion at 20° C. (completion monitored by HPLC). The reaction mixture was evaporated under reduced pressure. Residual diethylamine was removed by co-evaporations with portions of DMF/DCM and DCM. The residue was precipitated in DIPE. After filtration, the solid was washed with DIPE and dried under reduced pressure to yield 44.09 kg (100% recovery yield) of H-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 6) as a white powder with a purity of 87%.

### Example 10

### Solution Phase Synthesis of Fmoc-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 9)

H-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 6) (38.20 kg, 1 eq; as obtained in Example 9) was added to a mixture of HOBt (2.01 kg, 1 eq) and Fmoc--Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-OH (SEQ ID NO 8) (32.67 kg, 1 eq; as obtained in Example 3) in DMF (458 L). TOTU (5.00 kg, 1.1 eq) was added at −5° C. to the reaction mixture. The pH was adjusted to 6.5-7 with DIPEA and the temperature was allowed to rise up to 0° C. The completion of the reaction was monitored by HPLC.

The reaction mixture was precipitated in water. The solid was successively washed with water and DIPE. The final solid was dried under reduced pressure to yield 64.60 kg (96% recovery yield) of Fmoc-Ser(tBu)-Lys(Boc)-Gln(Trt)-Met-15Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 9) as a white powder with a purity of 80%.

### Example 11

### Solution Phase Synthesis of H-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 9)

Fmoc deprotection was accomplished analogously to Example 9 charging diethylamine (8.34 L, 6 eq), Fmoc-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 9) (64.56 kg, 1 eq; as obtained in Example 10) and DCM/DMF (4:1, v/v, 129 L) to yield 60.62 kg (98% recovery yield) of H-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 9) as a white powder with a purity of 78%.

### Example 12

### Solution Phase Synthesis of Boc-¹His(Trt)-Gly-Glu(OtBu)-Gly-⁵Thr(tBu)-Phe-Thr(tBu)-Ser(tBu)-Asp(OtBu)-¹⁰Leu-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 1)

H-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 9) (31.90 kg, 1 eq; as obtained in Example 11) was added to a mixture of HOBt (0.97 kg, 1 eq) and Boc-¹His(Trt)-Gly-Glu(OtBu)-Gly-⁵Thr(tBu)-Phe-Thr(tBu)-Ser(tBu)-Asp(OtBu)-¹⁰Leu-OH (SEQ ID NO 11) (11.29 kg, 1 eq; as obtained in Example 2) in NMP (226 L). PyBOP (3.48 kg, 1.3 eq) was added at −2° C. to the reaction mixture while adjusting the pH value to 6.5-7 with DIPEA. The reaction was allowed to go to completion (monitored by HPLC). The reaction mixture was precipitated in water. The solid was successively washed with water and DIPE and dried under reduced pressure to yield 41.93 kg (100% recovery yield) of Boc-¹His(Trt)-Gly-Glu(OtBu)-Gly-⁵Thr(tBu)-Phe-Thr(tBu)-Ser(tBu)-Asp(OtBu)-¹⁰Leu-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 1) as a white powder with a purity of 79%.

### Example 13

### Solution Phase Synthesis of H-¹His-Gly-Glu-Gly-⁵Thr-Phe-Thr-Ser-Asp-¹⁰Leu-Ser-Lys-Gln-Met-¹⁵Glu-Glu-Glu-Ala-Val-²⁰Arg-Leu-Phe-Ile-Glu-²⁵Trp-Leu-Lys-Asn-Gly-³⁰Gly-Pro-Ser-Ser-Gly-³⁵Ala-Pro-Pro-Pro-Ser-NH₂ (SEQ ID NO 1)

Boc-¹His(Trt)-Gly-Glu(OtBu)-Gly-⁵Thr(tBu)-Phe-Thr(tBu)-Ser(tBu)-Asp(OtBu)-¹⁰Leu-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 1) (41.88 kg, 1 eq; as obtained in Example 12) was slowly added at 15° C. to a mixture of TFA (235 L), TIS (6.28 L), water (6.28 L), EDT (6.3 L), ammonium iodide (1.21 kg, 1.25 eq) and DMS (0.62 L, 1.25 eq). The deprotection was allowed to go to completion at 18° C. (completion of cleavage monitored by HPLC). The reaction mixture was concentrated and the residue was precipitated in DIPE. After filtration, the solid was washed with DIPE and dried under reduced pressure to yield 35.06 kg (42% yield corrected by peptide content) of crude exenatide with a purity of 58% and a peptide content of 33%.

The crude product was purified by preparative HPLC on a C8 stationary phase (10 μm, 100 Å). In a first step the crude peptide was purified by gradient elution with TFA/H₂O/CH₃CN, in a second step by gradient elution with NH4HCO₃ in H₂O/CH₃CN, and finally in a third step by gradient elution with AcOH/H₂O/CH₃CN. The eluate fractions containing pure product were concentrated and desalted using HPLC on a C8 stationary phase (10 μm, 100 Å) and by step elution with AcOH/H₂O/CH₃CN. The fractions containing pure product were combined, concentrated and finally spray-dried (parameters: spray-dryer is Fujisaki MDL-050, exenatide solution is 40-50 g/L in water/1% AcOH/1% acetonitrile, flow rate (feed) is 2.0-2.4 kg/h, nitrogen temperature is 165-175° C., nitrogen flow rate is 1000-1200 L/min, product temperature (out of spray-dryer) is 40-50° C.), yielding 9.4 kg (78% yield corrected by peptide content) of purified H-¹His-Gly-Glu-Gly-⁵Thr-Phe-Thr-Ser-Asp-¹⁰Leu-Ser-Lys-Gln-Met-¹⁵Glu-Glu-Glu-Ala-Val-²⁰Arg-Leu-Phe-Ile-Glu-²⁵Trp-Leu-Lys-Asn-Gly-³⁰Gly-Pro-Ser-Ser-Gly-³⁵Ala-Pro-Pro-Pro-Ser-NH₂ (SEQ ID NO 1) as a white powder with 99.3% purity (by HPLC) and with a peptide content of 95.5%.

### Example 14

### Solution Phase Synthesis of H-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 6) [without isolation of the Fmoc-[22-39]-NH₂ intermediate (SEQ ID NO 6)]

TBTU (0.95 g, 1 eq) was added at −5° C. to a mixture of Fmoc-Phe-Ile-Glu(OtBu)-²⁵Trp(Boc)-Leu-Lys(Boc)-Asn(Trt)-Gly-OH (SEQ ID NO 3) (5.11 g, 1 eq; as obtained in Example 4) and HOBt (0.45 g, 1 eq) in NMP (24 mL). The pH was adjusted to 6.5-7 with DIPEA and the temperature was allowed to rise up to 0° C. After pre-activation of the Fmoc-[22-29]-OH (SEQ ID NO 3), a solution of H-³⁰Gly-Pro-Ser(tBu)-Ser(tBu)-Gly-³⁵Ala-Pro-Pro-Pro-Ser(tBu)-NH₂ (SEQ ID NO 4) (3.69 g, 1 eq; as obtained in Example 7) in NMP (91 L) was added at 0° C. and the reaction was allowed to go to completion at pH 6.5-7 and 0° C. (completion monitored by HPLC).

Once the reaction was complete, DCM (18 mL) was added and the temperature was adjusted to 20° C. Diethylamine (1.23 mL, 1 eq) was slowly added at least for 15 minutes and further stirred at 20° C. The reaction was stopped when the starting material Fmoc-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 6) was less than 0.5%. The reaction mixture was then concentrated under reduced pressure at a temperature below 25° C. Co-evaporation with DCM (1:4, v/v) was then carried out, in order to remove residual diethylamine. This operation was performed four times. The residual oil was then kept below 25° C. and MTBE was slowly added, whereupon the product precipitated. After completion of the addition, the suspension was stirred for 5 minutes, and then filtered off through a filter (14 μm pores) without vacuo. The filtration took 2 minutes which corresponds to a K value of 9.7. The residual solid was then washed (4 times) with DIPE. The filtrations of these 4 washings were straightforward (few seconds each).

After drying under reduced pressure, to yield 6.1 g (81% recovery yield) of H-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Pro-Ser(tBu)-NH₂ (SEQ ID NO 6) as a white powder with a purity of 88% (by HPLC).

### Example 15

### Solid Phase Synthesis of H-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 5)

The synthesis was performed in a similar way as described in Example 6b, respectively 6a and 2, using 40 g of Sieber amide resin (0.54 mol/kg) and 1.5 eq each of Fmoc-Lys(Boc)-OH, Fmoc-Ser(tBu)-OH, Fmoc-Ala-OH, Fmoc-Pro-OH and Fmoc-Gly-OH. In difference to Example 2, the peptide elongation was carried out with ethyl 2-cyano-2-hydroxyiminoacetate (OXYMAPURE®, 1.5 eq relative to the amino acid) instead of HOBt hydrate as the scavenger. At the end of the elongation, 404 g of wet protected peptidyl resin Fmoc-[30-44]-Sieber amide resin were obtained.

The further procedure was performed starting with 100 g portions of protected Fmoc-[30-44]-Sieber amide resin. The deprotection of the Fmoc group, the cleavage from the resin and the work-up procedures were accomplished similar to Example 6b.

Yield: 10.3 g of H-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 5) as a white solid with 78% purity and 84% recovery yield (based on a target batch size of 0.54 mmol).

### Example 16

### Solution Phase Synthesis of Fmoc-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 7)

Coupling between Fmoc-Phe-Ile-Glu(OtBu)-²⁵Trp(Boc)-Leu-Lys(Boc)-Asn(Trt)-Gly-OH (SEQ ID NO 3) (6.77 g, 0.9 eq; as obtained in Example 4) and H-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 5) (10 g, 1 eq; as obtained in Example 15) was performed in a similar way as described in Example 8.

Yield: 15.7 g (68% recovery yield, non-corrected) of Fmoc-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 7) as a white solid with 97% purity.

### Example 17

### Solution Phase Synthesis of H-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 7)

The Fmoc deprotection of Fmoc-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 7) (12.7 g; as obtained in Example 16) was performed in a similar way as described in Example 9, yielding 11.9 g (99% recovery yield) of H-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 7) as a white solid with 73% purity.

### Example 18

### Solution Phase Synthesis of Fmoc-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 10)

Coupling between Fmoc-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-OH (SEQ ID NO 8) (7.5 g, 0.88 eq; as obtained in Example 3) and H-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 7) (13.6 g, 1 eq; as obtained in Example 17) was performed in a similar way as described in Example 10, yielding 19.7 g (89% recovery yield) of Fmoc-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 10) as a white solid with 71% purity.

### Example 19

### Solution Phase Synthesis of H-Ser(tBu)-Lys(Boc)-Gln(Trt)-Met-¹⁵Glu(OtBu)-Glu(OtBu)-Glu(OtBu)-Ala-Val-20Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 10)

The Fmoc deprotection of Fmoc-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 10) (19.2 g; as obtained in Example 18) was performed in a similar way as described in Example 11, yielding 19.8 g (quantitative recovery yield) of H-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 10) as a white solid with 66% purity.

### Example 20

### Solution Phase Synthesis of Boc-¹His(Trt)-Gly-Glu(OtBu)-Gly-⁵Thr(tBu)-Phe-Thr(tBu)-Ser(tBu)-Asp(OtBu)-¹⁰Leu-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 2)

Coupling between Boc-¹His(Trt)-Gly-Glu(OtBu)-Gly-⁵Thr(tBu)-Phe-Thr(tBu)-Ser(tBu)-Asp(OtBu)-¹⁰Leu-OH (SEQ ID NO 11) (5.2 g, 1 eq; as obtained in Example 2) and H-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-²²Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 10) (18.2 g, 1 eq; as obtained in Example 19) was performed in a similar way as described in Example 12, yielding 22.9 g (97% recovery yield) of Boc-¹His(Trt)-Gly-Glu(OtBu)-Gly-⁵Thr(tBu)-Phe-Thr(tBu)-Ser(tBu)-Asp(OtBu)-¹⁰Leu-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 2) as a white solid with a purity of 76%.

### Example 21

### Solution Phase Synthesis of H-¹His-Gly-Glu-Gly-⁵Thr-Phe-Thr-Ser-Asp-¹⁰Leu-Ser-Lys-Gln-Met-¹⁵Glu-Glu-Glu-Ala-Val-²⁰Arg-Leu-Phe-Ile-Glu-²⁵Trp-Leu-Lys-Asn-Gly-³⁰Gly-Pro-Ser-Ser-Gly-³⁵Ala-Pro-Pro-Ser-Lys-⁴⁰Lys-Lys-Lys-Lys-Lys-N H₂ (SEQ ID NO 2)

Global deprotection of Boc-¹His(Trt)-Gly-Glu(OtBu)-Gly-⁵Thr(tBu)-Phe-Thr(tBu)-Ser(tBu)-Asp(OtBu)-¹⁰Leu-Ser(tBu)-Lys(Boc)-Gln(Trt)-Met-¹⁵Glu(OtBu)-Glu(OtBu)-Glu(OtBu)-Ala-Val-²⁰Arg(Pbf)-Leu-Phe-Ile-Glu(OtBu)-²⁵Trp(Boc)-Leu-Lys(Boc)-Asn(Trt)-Gly-³⁰Gly-Pro-Ser(tBu)-Ser(tBu)-Gly-³⁵Ala-Pro-Pro-Ser(tBu)-Lys(Boc)-⁴⁰Lys(Boc)-Lys(Boc)-Lys(Boc)-Lys(Boc)-Lys(Boc)-NH₂ (SEQ ID NO 2) (as obtained in

Example 20) was performed in a similar way as described in Example 13, using a deprotection mixture of TFA (124 mL), TIS (3.4 mL), water (3.4 mL), EDT (3.4 mL), ammonium iodide (527.7 mg, 1.25 eq) and DMS (0.3 mL, 1.25 eq). Yield: 19.9 g of crude exenatide. The crude product was purified by preparative HPLC on a C18 stationary phase (10 μm, 100 Å). In a first step the crude peptide (11 g) was purified by gradient elution with TFA/H₂O/CH₃CN, in a second step by gradient elution with AcOH/H₂O/CH₃CN, and finally concentrated and desalted by gradient elution with AcOH/H₂O/CH₃CN. The fractions containing pure product were combined, concentrated by evaporation and lyophilised, yielding 0.61 g of purified H-¹His-Gly-Glu-Gly-⁵Thr-Phe-Thr-Ser-Asp-¹⁰Leu-Ser-Lys-Gln-Met-¹⁵Glu-Glu-Glu-Ala-Val-²⁰Arg-Leu-Phe-Ile-Glu-²⁵Trp-Leu-Lys-Asn-Gly-³⁰Gly-Pro-Ser-Ser-Gly-³⁵Ala-Pro-Pro-Ser-Lys-⁴⁰Lys-Lys-Lys-Lys-Lys-NH₂ (SEQ ID NO 2) as a white powder with 98.1% purity (by HPLC).

### Example 22a

### Solid Phase Synthesis of Fmoc-²²Phe-Ile-Glu(tBu)-Trp(Boc)-Leu-²⁷Lys(Boc)-Asn(Trt)-Gly-³⁰Gly-OH (SEQ ID NO 17)

The first steps of synthesis were carried out manually in a 60 ml solid phase reactor. Fmoc-Gly-OH (0.445 g, 0.5 eq) was loaded onto CTC resin (3 g, 1.55 mmol/g) in the presence of DIPEA (2.55 ml, 10.0 eq relative to the amino acid) in DCM (minimum quantity to solve the amino acid, 4 ml). The reaction was finished after 60 minutes. After completion, the unreacted active positions on the resin were then capped by reaction with methanol (2.4 ml) added directly to the reaction mixture. The bed was drained, and thoroughly washed with DCM (3 times 30 ml) and DMF (3 times 30 ml).

Removal of the Fmoc group was accomplished at room temperature by using a solution of piperidine in DMF (20% piperidine; 1 time 1 min; 2 times 5 min; 30 ml each); four to five cycles were carried out. The bed was drained, and the H-Gly-CTC resin obtained was washed with DMF (3 times 30 ml) and DCM (3 times 30 ml) to remove residual piperidine. The dibenzofulvene formed as a product of Fmoc removal was quantified by UV (0.47 mmol/g)

The following steps to complete the peptide synthesis were performed in a mid-scale peptide synthesizer automated solid phase synthesis. The equipment runs using the same standard conditions to introduce every amino acid. A solution of Fmoc-Gly-OH (4.460 g, 5.0 eq) in DMF (20 ml) was added, and the coupling accomplished at room temperature in the presence of HBTU (5.689 g, 5.0 eq) and DIPEA (10.0 eq, pH=7-8). The coupling reaction time was fixed in 60 minutes at room temperature. After complete coupling, the obtained Fmoc-Gly-Gly-CTC resin was drained and thoroughly washed with DMF (3 times 30 ml) and DCM (3 times 30 ml).

The Fmoc group was removed as described after the introduction of the first amino acid on the resin. Fmoc-Asn(Trt)-OH (9.010 g, 5.0 eq), which was the subsequent amino acid in the sequence, was dissolved in DMF (10 ml) and transferred to the H-Gly-Gly-CTC resin, HBTU (5.689 g, 5.0 eq relative to the amino acid) solved in DMF (20 ml) and DIPEA (10.0 eq, pH=7-8) in DMF (10 ml) were added consecutively to the reaction mixture. The coupling reaction was accomplished in 60 minutes at room temperature. After complete coupling, the peptidyl resin was drained and thoroughly washed with DMF (3 times 30 ml) and DCM (3 times 30 ml).

The elongation cycle (Fmoc removal and amino acid coupling with coupling reagent HBTU (5.689 g, 5.0 eq) and DIPEA (10.0 eq, pH=7-8) was repeated for subsequent assembly of the peptide fragment using 5.0 eq each of Fmoc-Lys(Boc)-OH, Fmoc-Leu-OH, Fmoc-Trp(Boc)-OH, Fmoc-Glu(tBu)-OH, Fmoc-Ile-OH and Fmoc-Phe-OH.

After the last elongation cycle, the protected peptide was washed with DCM (3 times 30 ml) and cleaved from the resin by adding 2% TFA in DCM (8 times 4 ml). After filtration, the solution of the peptide was evaporated partially under reduced pressure and the peptide was precipitated with diethyl ether. Centrifugation afforded the solid peptide that was successively washed with diethyl ether (20 ml per 250 mg of peptide). Three to four cycles of ether washes were carried out. After the diethyl ether washes the solid was dissolved in ACN—H2O (1:1) and lyophilized affording 1.99 g of Fmoc-²²Phe-Ile-Glu(tBu)-Trp(Boc)-Leu-²⁷Lys(Boc)-Asn(Trt)-Gly-³⁸Gly-H (SEQ ID NO 17) as a white to beige powder with 95.97% purity.

### Example 22b

### Solid Phase Synthesis of Fmoc-³¹Pro-Ser(tBu)-Ser(tBu)-Gly-³⁵Ala-Pro-Pro-³⁸Pro-OH (SEQ ID NO 25)

The first steps of the synthesis were carried out manually in a 60 ml solid phase reactor. Fmoc-Pro-OH (0.533 g, 0.5 eq) was loaded onto CTC resin (3 g, 1.55 mmol/g) in presence of DIPEA (2.55 ml, 10.0 eq relative to the amino acid) in DCM (minimum quantity to solve the amino acid, 4 ml). After completion, the unreacted active positions on the resin were then capped by reaction with methanol (0.8 ml) added directly to the reaction mixture. The bed was drained, and thoroughly washed with DCM (3 times 30 ml) and DMF (3 times 30 ml).

Removal of the Fmoc group was accomplished at room temperature by using a solution of piperidine in DMF (20% piperidine; 1 time 1 min; 2 times 5 min; 30 ml each); four to five cycles were carried out. The bed was drained, and the H-Pro-CTC resin obtained was washed with DMF (3 times 30 ml) and DCM (3 times 30 ml) to remove residual piperidine. The dibenzofulvene formed as a product of Fmoc removal was quantified by UV and its value is directly related with the real loading of resin (0.51 mmol/g).

The following steps to complete the peptide synthesis were performed in a mid-scale peptide synthesizer automated solid phase synthesis. The equipment runs using the same standard conditions to introduce every amino acid. A solution of Fmoc-Pro-OH (5.061 g, 5.0 eq) in DMF (20 ml) was added, and the coupling accomplished at room temperature in the presence of HBTU (5.689 g, 5.0 eq) and DIPEA (10.0 eq, pH=7-8). The coupling reaction time was fixed in 60 minutes at room temperature. After complete coupling, the obtained Fmoc-Pro-Pro-CTC resin was drained and thoroughly washed with DMF (3 times 30 ml) and DCM (3 times 30 ml).

The Fmoc group was removed as described after the introduction of the first amino acid on the resin, Fmoc-Pro-OH (5.061 g, 5.0 eq), which was the subsequent amino acid in the sequence, were dissolved in DMF (5 ml) and transferred to the H-Pro-Pro-CTC resin, HBTU (5.689 g, 5.0 eq relative to the amino acid) solved in DMF (20ml) and DIPEA (10.0 eq, pH=7-8). The coupling reaction was accomplished in 60 minutes at room temperature. After complete coupling, the peptidyl resin was drained and thoroughly washed with DMF (3 times 30 ml) and DCM (3 times 30 ml).

The elongation cycle (Fmoc deprotection, amino acid coupling with coupling reagent HBTU (5.689 g, 5.0 eq) and DIPEA (10.0 eq)), was repeated for subsequent assembly of the peptide fragment using 5.0 eq each of Fmoc-Ala-OH, Fmoc-Gly-OH, Fmoc-Ser(tBu)-OH, Fmoc-Ser(tBu)-OH and Fmoc-Pro-OH.

After the last elongation cycle, the protected peptide was washed with DCM (3 times 30 ml) and cleaved from the resin by adding 2% TFA in DCM (8 times 4 ml). After filtration, the cleavage solution was evaporated partially under reduced pressure and precipitated with diethyl ether. The centrifugation step afforded the solid peptide which was successively washed with diethyl ether. After the diethyl ether washes the solid was dissolved in ACN—H₂O (1:1) and lyophilized affording 0.88 g of Fmoc-³¹Pro-Ser(tBu)-Ser(tBu)-Gly-³⁵Ala-Pro-Pro-³⁸Pro-H (SEQ ID NO 25) as a white to beige powder with 91.67% purity.

### Example 22c

### Solution Phase Synthesis of Fmoc-³¹Pro-Ser(tBu)-Ser(tBu)-Gly-³⁵Ala-Pro-Pro-Pro-³⁹Ser(tBu)-NH₂ (SEQ ID NO 18)

HBTU (40 mg, 1.1 eq) and DIPEA (31.6 microliter, 2.0 eq) were added to Fmoc-³¹Pro-Ser(tBu)-Ser(tBu)-Gly-³⁵Ala-Pro-Pro-Pro-OH (SEQ ID NO 25) (100 mg, 1.0 eq; prepared according to example 22b) in DCM (1.5 ml). After pre-activation, the H-³⁹Ser(tBu)-NH₂ (16.8 mg, 1.1 eq) was added at room temperature. The completion of the reaction was monitored by HPLC and the reaction was finished after 2 hours.

The solution is extracted consecutively with 1N HCl (3 times 10 ml), H₂O (3 times 10 ml), sodium bicarbonate (3 times 10 ml) and H₂O (3 times 10 ml). The organic phase is evaporated under reduced pressure, solved and lyophilized to yield 113.0 mg (quantitative yield) of Fmoc-³¹Pro-Ser(tBu)-Ser(tBu)-Gly-³⁵Ala-Pro-Pro-Pro-³⁹Ser(tBu)-NH₂ (SEQ ID NO 18) as a white powder with a purity of 81%.

The same protocol followed on a 400 mg scale of Fmoc-³¹Pro-Ser(tBu)-Ser(tBu)-Gly-³⁵Ala-Pro-Pro-Pro-OH (SEQ ID NO 25) was followed and the same results were obtained. It is therefore possible to produce more quantity obtaining the same results.

### Example 22d

### Solution Phase Synthesis of H-³¹Pro-Ser(tBu)-Ser(tBu)-Gly-³⁵Ala-Pro-Pro-Pro-³⁹Ser(tBu)-NH₂ (SEQ ID NO 18)

Diethylamine (0.380 ml, 4.5 eq) was slowly added at room temperature to a solution of Fmoc-³¹Pro-Ser(tBu)-Ser(tBu)-Gly-³⁵Ala-Pro-Pro-Pro-³⁹Ser(tBu)-NH₂ (SEQ ID NO 18) (255.6 mg, 1.0 eq; prepared according to example 22c) in DCM (2 ml) and the reaction was allowed to go to completion at room temperature (monitored by HPLC). The reaction was finished within 2 hours and the reaction mixture was evaporated under reduced pressure and co-evaporations with toluene (three to four toluene cycles were carried out). The solid was solved ACN—H₂O (1:1) and lyophilized to yield 204.7 mg (99% recovery yield) of H-³¹Pro-Ser(tBu)-Ser(tBu)-Gly-³⁵Ala-Pro-Pro-Pro-³⁹Ser(tBu)-NH₂ as a white powder with a purity of 95.0%.

### Example 22e

### Solution Phase Synthesis of Fmoc-²²Phe-Ile-Glu(tBu)-Trp(Boc)-Leu-²⁷Lys(Boc)-Asn(Trt)-Gly-Gly-Pro-³²Ser(tBu)-Ser(tBu)-Gly-Ala-³⁶Pro-Pro-Pro-³⁹Ser(tBu)-NH₂ (SEQ ID NO 6)

The peptide fragment Fmoc-²²Phe-Ile-Glu(tBu)-Trp(Boc)-Leu-²⁷Lys(Boc)-Asn(Trt)-Gly-³⁰Gly-OH and HOBt (17.2 mg, 1.0 eq; prepared according to example 22a) were solved in DMF (1.5 ml). The PyBOP (75.9 mg, 1.3 eq) solved in DMF (0.2 ml) was added to the reaction mixture at low temperature and the pH was adjusted to 8-9 with DIPEA. After 5 minutes of acid activation, the DMF (0.2 ml) solved peptide fragment H-³¹Pro-Ser(tBu)-Ser(tBu)-Gly-³⁵Ala-Pro-Pro-Pro-³⁹Ser(tBu)-NH₂ (SEQ ID NO 18) (107.9 mg, 1.0 eq; prepared according to example 22d) was added to the reaction mixture. The temperature of reaction was controlled to 0-5° C. for first 4 hours and then the reaction was allowed to go to completion at room temperature. The reaction was monitored by HPLC and 20 hours was the total reaction time. After precipitating the protected peptide in H₂O and washing with diethyl ether the solid was solved ACN—H₂O (1:1) and lyophilized to yield 302.7 (99% recovery yield) of Fmoc-²²Phe-Ile-Glu(tBu)-Trp(Boc)-Leu-²⁷Lys(Boc)-Asn(Trt)-Gly-Gly-Pro-³²Ser(tBu)-Ser(tBu)-Gly-Ala-³⁶Pro-Pro-Pro-³⁹Ser(tBu)-NH₂ as a white powder with a purity of 77.7%.

### Example 23a

### Solid Phase Synthesis of Fmoc-²²Phe-Ile-Glu(tBu)-Trp(Boc)-Leu-²⁷Lys(Boc)-Asn(Trt)-Gly-Gly-³¹Pro-OH (SEQ ID NO 19)

The first steps of synthesis were carried out manually in a 60 ml solid phase reactor. Fmoc-Pro-OH (0.533 g, 0.5 eq) was loaded onto CTC resin (3 g, 1.55 mmol/g) in presence of DIPEA (2.55 ml, 10.0 eq relative to the amino acid) in DCM (minimum quantity to solve the amino acid, 4 ml). The reaction was finished after 60 minutes. After completion, the unreacted active positions on the resin were then capped by reaction with methanol (2.4 ml) added directly to the reaction mixture. The bed was drained, and thoroughly washed with DCM (3 times 30 ml) and DMF (3 times 30 ml).

Removal of the Fmoc group was accomplished at room temperature by using a solution of piperidine in DMF (20% piperidine; 1 time 1 min; 2 times 5 min; 30 ml each); four to five cycles were carried out. The bed was drained, and the H-Gly-CTC resin obtained was washed with DMF (3 times 30 ml) and DCM (3 times 30 ml) to remove residual piperidine. The dibenzofulvene formed as a product of Fmoc removal was quantified by (0.50 mmol/g).

The following steps to complete the peptide synthesis were performed in a mid-scale peptide synthesizer automated solid phase synthesis. The equipment runs using the same standard conditions to introduce every amino acid. A solution of Fmoc-Gly-OH (4.46 g, 5.0 eq) in DMF (20 ml) was added, and the coupling accomplished at room temperature in the presence of HBTU (5.689 g, 5.0 eq) and DIPEA (10.0 eq, pH=7-8). The coupling reaction time was fixed in 60 minutes at room temperature. After complete coupling, the obtained Fmoc-Gly-Pro-CTC resin was drained and thoroughly washed with DMF (3 times 30 ml) and DCM (3 times 30 ml).

The Fmoc group was removed as described after the introduction of the first amino acid on the resin.

The elongation cycle (Fmoc removal and amino acid coupling with coupling reagent HBTU (5.689 g, 5.0 eq) and DIPEA (10.0 eq)) was repeated for subsequent assembly of the peptide fragment using 5.0 eq each of Fmoc-Gly-OH, Fmoc-Asn(Trt)-OH, Fmoc-Lys(Boc)-OH, Fmoc-Leu-OH, Fmoc-Trp(Boc)-OH, Fmoc-Glu(tBu)-OH, Fmoc-Ile-OH and Fmoc-Phe-OH.

After the last elongation cycle, the protected peptide was washed with DCM (3 times 30 ml) and cleaved from the resin by adding 2% TFA in DCM (8 times 4 ml). Two cycles were carried out and collected separately. After filtration, the solution of the peptide was evaporated partially under reduced pressure and the peptide was precipitated with diethyl ether. Centrifugation afforded the solid peptide which was successively washed with diethyl ether. After the diethyl ether washes the solid was dissolved in ACN—H₂O (1:1) and lyophilized affording 2.30 g of Fmoc-22Phe-Ile-Glu(tBu)-Trp(Boc)-Leu-²⁷Lys(Boc)-Asn(Trt)-Gly-Gly-³¹Pro-OH (SEQ ID NO 19) as a white to beige powder with 95.97% purity.

### Example 23b

### Solid Phase Synthesis of Fmoc-³²Ser(tBu)-Ser(tBu)-Gly-Ala-Pro-Pro-³⁸Pro-OH (SEQ ID NO 26)

The first steps of synthesis were carried out manually in a 60 ml solid phase reactor. Fmoc-Pro-OH (1.066 g, 1.0 eq) was loaded onto CTC resin (3 g, 1.55 mmol/g) in presence of DIPEA (9.92 ml, 10.0 eq relative to the amino acid) in DCM (minimum quantity to solve the amino acid, 8 ml). The reaction was finished after 60 minutes. After completion, the unreacted active positions on the resin were then capped by reaction with excess of methanol (2.4 ml) added directly to the reaction mixture. The bed was drained, and thoroughly washed with DCM (3 times 30 ml) and DMF (3 times 30 ml). Removal of the Fmoc group was accomplished at room temperature by using a solution of piperidine in DMF (20% piperidine; 1 time 1 min; 2 times 5 min; 30 ml each); four to five cycles were carried out. The bed was drained, and the H-Pro-CTC resin obtained was washed with DMF (3 times 30 ml) and DCM (3 times 30 ml) to remove residual piperidine. The dibenzofulvene formed as a product of Fmoc removal was quantified by UV and its value is directly related with the real loading of resin (1 mmol/g).

The following steps to complete the peptide synthesis were performed in a mid-scale peptide synthesizer automated solid phase synthesis. The equipment runs using the same standard conditions to introduce every amino acid. A solution of Fmoc-Pro-OH (5.06 g, 5.0 eq) in DMF (20 ml) was added, and the coupling accomplished at room temperature in the presence of TBTU (4.816 g, 5.0 eq) and DIPEA (10.0 eq, pH=7-8). The coupling reaction time was fixed in 60 minutes at room temperature. After complete coupling, the obtained Fmoc-Pro-Pro-CTC resin was drained and thoroughly washed with DMF (3 times 30 ml) and DCM (3 times 30 ml).

The Fmoc group was removed as described after the introduction of the first amino acid on the resin.

The elongation cycle (Fmoc removal and amino acid coupling with coupling reagent TBTU (4.816 g, 5.0 eq) and DIPEA (10.0 eq)) was repeated for subsequent assembly of the peptide fragment using 5.0 eq each of Fmoc-Pro-OH, Fmoc-Ala-OH, Fmoc-Gly-OH, Fmoc-Ser(tBu)-OH and Fmoc-Ser(tBu)-OH.

After the last elongation cycle, the protected peptide was washed with DCM (3 times 30 ml) and cleaved from the resin by adding 2% TFA in DCM (8 times 4 ml). After filtration, the solution of the peptide was evaporated partially under reduced pressure and the peptide was precipitated with diethyl ether. Centrifugation afforded the solid peptide which was successively washed with diethyl ether. After the diethyl ether washes the solid was dissolved in ACN—H₂O (1:1) and lyophilized affording 2.1 g of Fmoc-³²Ser(tBu)-Ser(tBu)-Gly-Ala-Pro-Pro-³⁸Pro-OH (SEQ ID NO 26) as a white to beige powder with 62.77% purity.

### Example 23c

### Solution Phase Synthesis of Fmoc-³²Ser(tBu)-Ser(tBu)-Gly-³⁵Ala-Pro-Pro-Pro-³⁹Ser(tBu)-NH₂ (SEQ ID NO 20)

HBTU (44.1 mg, 1.1 eq) and DIPEA (44 microliter, 2.0 eq) were added to Fmoc-³²Ser(tBu)-Ser(tBu)-Gly-³⁵Ala-Pro-Pro-³⁸Pro-OH (SEQ ID NO 26) (100 mg, 1.0 eq; prepared according to example 23b) in DCM (1 ml). After pre-activation, the H-³⁹Ser(tBu)-NH₂ (18.6 mg, 1.1 eq) was added at room temperature. The pH was adjusted to pH 8-9 with DIPEA. The completion of the reaction was monitored by HPLC and the reaction was finished after 48 hours.

The solution is extracted consecutively with three times with 1N HCl (3 times 10 ml), H₂O (3 times 10 ml), sodium bicarbonate (3 times 10 ml) and H₂O (3 times 10 ml). The organic phase is evaporated under reduced pressure, solved and lyophilized to yield 252 mg (82% recovery yield) of Fmoc-³²Ser(tBu)-Ser(tBu)-Gly-³⁵Ala-Pro-Pro-Pro-³⁹Ser(tBu)-NH₂ (SEQ ID NO 20) as a white powder with a purity of 100%.

### Example 23d

### Solution Phase Synthesis of H-³²Ser(tBu)-Ser(tBu)-Gly-³⁵Ala-Pro-Pro-Pro-³⁹Ser(tBu)-NH₂ (SEQ ID NO 20)

Diethylamine (0.3805 ml, 13.0 eq) was slowly added at room temperature to a solution of Fmoc-³²Ser(tBu)-Ser(tBu)-Gly-³⁵Ala-Pro-Pro-Pro-³⁹Ser(tBu)-NH₂ (SEQ ID NO 20) (267.2 mg, 1.0 eq; prepared according to example 23c) in DCM (2 ml) and the reaction was allowed to go to completion at room temperature (monitored by HPLC). The reaction was finished within 2 hours and the reaction mixture was evaporated under reduced pressure and co-evaporations with toluene (three to four toluene cycles were carried out). The solid was solved ACN—H₂O (1:1) and lyophilized to yield 212.6 mg (quantitative yield) of H-³²Ser(tBu)-Ser(tBu)-Gly-³⁵Ala-Pro-Pro-Pro-³⁹Ser(tBu)-NH₂ as a white powder with a purity of 100%.

### Example 23e

### Solution Phase Synthesis of Fmoc-²²Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 6)

The peptide fragment Fmoc-²²Phe-Ile-Glu(tBu)-Trp(Boc)-Leu-²⁷Lys(Boc)-Asn(Trt)-Gly-Gly-³¹Pro-OH prepared according to example 23a and HOBt (16.3 mg , 1.0 eq) were solved in DMF (1.5 ml). The PyBOP (72 mg, 1.3 eq) solved in DMF (0.2 ml) was added to the reaction mixture at low temperature and the pH was adjusted to 8-9 with DIPEA. After 5 minutes of acid activation, the DMF (0.2 ml) solved peptide fragment H-³²Ser(tBu)-Ser(tBu)-Gly-³⁵Ala-Pro-Pro-Pro-³⁹Ser(tBu)-NH₂ (SEQ ID NO 20) (92 mg, 1.0 eq; prepared according to example 23d) was added to the reaction mixture. The temperature of reaction was controlled to 0-5° C. for first 4h and then the reaction was allowed to go to completion at room temperature. The reaction was monitored by HPLC and 20 hours was the total reaction time. After precipitating the protected peptide in H₂O and washing with diethyl ether the solid was solved ACN—H₂O (1:1) and lyophilized to yield 287.4 mg (99% recovery yield) Fmoc-22Phe-Ile-Glu(tBu)-Trp(Boc)-Leu-27Lys(Boc)-Asn(Trt)-Gly-Gly-Pro-³²Ser(tBu)-Ser(tBu)-Gly-Ala-³⁶Pro-Pro-Pro-³⁹Ser(tBu)-NH₂ as a white powder with a purity of 89.5%.

### Comparative Example 24a

### Solid Phase Synthesis of Fmoc-¹³G1n(Trt)-Met-Glu(tBu)-Glu(tBu)-Glu(tBu)-¹⁸Ala-Val-Arg(Pbf)-Leu-Phe-Ile-Glu(tBu)-²⁶Trp(Boc)-Leu-Lys(Boc)-Asn(Trt)-²⁹Gly-OH (SEQ ID NO 27)

The first steps of synthesis were carried out manually in a 60 ml solid phase reactor. Fmoc-Gly-OH (0.713 g, 0.8 eq) was loaded onto CTC resin (3 g, 1.55 mmol/g) in presence of DIPEA (4.96 ml, 10.0 eq relative to the amino acid) in DCM (minimum quantity to solve the amino acid, 4 ml). The reaction was finished after 60 minutes. After completion, the unreacted active positions on the resin were then capped by reaction with excess of methanol (2.4 ml) added directly to the reaction mixture. The bed was drained, and thoroughly washed with DCM (3 times 30 ml) and DMF (3 times 30 ml). Removal of the Fmoc group was accomplished at room temperature by using a solution of piperidine in DMF (20% piperidine; 1 time 1 min; 2 times 5 min; 30 ml each); four to five cycles were carried out. The bed was drained, and the H-Gly-CTC resin obtained was washed with DMF (3 times 30 ml) and DCM (3 times 30 ml) to remove residual piperidine. The dibenzofulvene formed as a product of Fmoc removal was quantified by UV and its value is directly related with the real loading of resin (0.8 mmol/g).

The following steps to complete the peptide synthesis were performed in a mid-scale peptide synthesizer automated solid phase synthesis. The equipment runs using the same standard conditions to introduce every amino acid. A solution of Fmoc-Asn(Trt)-OH (7.16 g, 5.0 eq) in DMF (20 ml) was added, and the coupling accomplished at room temperature in the presence of HBTU (5.689 g, 5.0 eq) and DIPEA (10.0 eq, pH=7-8). The coupling reaction time was fixed in 60 minutes at room temperature. After complete coupling, the obtained Fmoc-Asn(Trt)-Gly-CTC resin was drained and thoroughly washed with DMF (3 times 30 ml) and DCM (3 times 30 ml).

The Fmoc group was removed as described after the introduction of the first amino acid on the resin.

The elongation cycle (Fmoc removal and amino acid coupling with coupling reagent HBTU (5.689 g, 5.0 eq) and DIPEA (10.0 eq)) was repeated for subsequent assembly of the peptide fragment using 5.0 eq each of Fmoc-Lys(Boc)-OH, Fmoc-Leu-OH, Fmoc-Trp(Boc)-OH, Fmoc-Glu(tBu)-OH, Fmoc-Ile-OH, Fmoc-Phe-OH, Fmoc-Leu-OH, Fmoc-Arg(Pbf)-OH, Fmoc-Val-OH, Fmoc-Ala-OH, Fmoc-Glu(tBu)-OH, Fmoc-Glu(tBu)-OH, Fmoc-Glu(tBu)-OH, Fmoc-Met-OH and Fmoc-Gln(Trt)-OH.

After the last elongation cycle, the protected peptide was washed with DCM (3 times 30 ml) and cleaved from the resin by adding 2% TFA in DCM (8 times 4 ml). After filtration, the solution of the peptide was evaporated partially under reduced pressure and the peptide was precipitated with diethyl ether. Centrifugation afforded the solid peptide which was successively washed with diethyl ether. After the diethyl ether washes the solid was dissolved in ACN—H₂O (1:1) and lyophilized affording 2.70 g of Fmoc-¹³Gln(Trt)-Met-Glu(tBu)-Glu(tBu)-Glu(tBu)-Ala-¹⁹Val-Arg(Pbf)-Leu-Phe-Ile-Glu(tBu)-²⁵Trp(Boc)-Leu-Lys(Boc)-Asn(Trt)-²⁹Gly-OH (SEQ ID NO 27) as a white to beige powder with 85.74% purity. This low purity was due to steric hindrance of amino acids from sequence [¹³G1n(Trt)-Met-Glu(tBu)-Glu(tBu)-¹⁷Glu(tBu)], these difficult couplings were confirmed in manual synthesis of example 25b.

### Comparative Example 24b

### Solid Phase Synthesis of Boc-¹His(Trt)-Gly-Glu(tBu)-⁴Gly-PsiThr-Phe-Thr(tBu)-⁸Ser(tBu)-Asp(tBu)-Leu-PsiSer-¹²Lys(Boc)-OH (SEQ ID NO 28)

The synthesis was carried out in a 60 ml solid phase reactor. Fmoc-Lys(Boc)-OH (1.400 g, 0.5 eq) was loaded onto CTC resin (6 g, 1.55 mmol/g) in presence of DIPEA (4.96 ml, 10.0 eq relative to the amino acid) in DCM (minimum quantity to solve the amino acid, 9 ml). The reaction was finished after 60 minutes. After completion, the unreacted active positions on the resin were then capped by reaction with excess of methanol (4.8 ml) added directly to the reaction mixture. The bed was drained, and thoroughly washed with DCM (3 times 40 ml) and DMF (3 times 40 ml).

Removal of the Fmoc group was accomplished at room temperature by using a solution of piperidine in DMF (20% piperidine; 1 time 1 min; 2 times 5 min; 40 ml each); four to five cycles were carried out. The bed was drained, and the H-Lys(Boc)-CTC resin obtained was washed with DMF (3 times 40 ml) and DCM (3 times 40 ml) to remove residual piperidine. The dibenzofulvene formed as a product of Fmoc removal was quantified by UV and its value is directly related with the real loading of resin (0.42 mmol/g).

A solution of pseudoproline Fmoc-Leu-PsiSer-OH (3.60 g, 3.0 eq) in DMF (3 ml) was added, and the coupling accomplished room temperature in the presence of oxima (1.07 g, 3.0 eq) and DIPCDI (1.171 ml, 3.0 eq). The coupling reaction time was increased to 16h to introduce the pseudoproline quantitatively. Its completeness was determined by the ninhydrin test. After complete coupling, the obtained Fmoc-(Leu-PsiSer)-Lys(Boc)-CTC resin was drained and thoroughly washed with DMF (3 times 40 ml) and DCM (3 times 40 ml).

The Fmoc group was removed as described after the introduction of the first amino acid on the resin.

A solution of Fmoc-Asp(tBu)-OH (3.10 g, 3.0 eq) in DMF (3 ml) was added, and the coupling accomplished at room temperature in the presence of HOBt (1.158 g, 3.0 eq) and DIPCDI (1.171 ml, 3.0 eq). The coupling reaction time was 60 minutes and its completeness was determined by the ninhydrin test. After complete coupling, the obtained Fmoc-Asp(tBu)-(Leu-PsiSer)-Lys(Boc)-CTC resin was drained and thoroughly washed with DMF (3 times 40 ml) and DCM (3 times 40 ml).

The Fmoc group was removed as described after the introduction of the first amino acid on the resin.

The elongation cycle Fmoc removal, amino acid coupling with coupling reagent HOBt (1.158 g, 3.0 eq) and DIPCDI (1.171 ml, 3.0 eq) was repeated for subsequent assembly of the peptide fragment using 3.0 eq each of Fmoc-Ser(tBu)-OH, Fmoc-Thr(tBu)-OH, Fmoc-Phe-OH, Fmoc-Gly-PsiThr-OH, Fmoc-Glu(tBu)-OH, Fmoc-Gly-OH and Boc-His(Trt)-OH.

The coupling conditions to introduce the other pseudoproline Fmoc-Gly-PsiThr-OH were the same used for the other pseudoproline, 3.0 eq of amino acid, oxima (1.07 g, 3.0 eq) and DIPCDI (1.171 ml, 3.0 eq).

After the last elongation cycle, the protected peptide was washed with DCM (3 times 40 ml) and cleaved from the resin by adding 1% TFA in DCM (17 times 4 ml). Two cycles were carried out. After filtration, the solution of the peptide was neutralized to pH 7 (saturated solution of NH4HCO₃ in H2O). The resulted solution was evaporated under reduced pressure and solved in ACN—H₂O (1:1) and lyophilized. The fragment peptide Boc-¹His(Trt)-Gly-Glu(tBu)-⁴Gly-PsiThr-Phe-Thr(tBu)-⁸Ser(tBu)-Asp(tBu)-Leu-PsiSer-¹²Lys(Boc)-OH (SEQ ID NO 28) was obtained as a white to beige powder (4.9 g) with 100% purity.

### Comparative Example 24c

### Solution Phase Synthesis of Fmoc-¹³G1n(Trt)-Met-Glu(tBu)-Glu(tBu)-Glu(tBu)-¹⁵Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 29)

TBTU (110.2 mg, 1.0 eq) was added at −5° C. to a mixture of Fmoc-¹³Gln(Trt)-Met-Glu(tBu)-Glu(tBu)-Glu(tBu)-¹⁵Ala-Val-Arg(Pbf)-Leu-Phe-Ile-Glu(tBu)-²⁵Trp(Boc)-Leu-Lys(Boc)-Asn(Trt)-²⁹Gly-OH (SEQ ID NO 27) (1.19 g, 1.0 eq; prepared according to example 24a) and HOBt (52.54 mg, 1.0 eq) in NMP (7 ml). The pH was adjusted to 8-9 with DIPEA. After pre-activation of that peptide fragment, a solution of H-³⁰Gly-Pro-Ser(tBu)-Ser(tBu)-Gly-³⁵Ala-Pro-Pro-Pro-Ser(tBu)-NH₂ (350mg, 1.0 eq; prepared according to either example 6a or 6b) in NMP (4 ml) was added at 0-3° C. and the reaction was controlled at this temperatures and pH 8-9. After 4 hours the reaction was allowed to rise up to room temperature. The reaction was monitored by HPLC and 24 hours was the total reaction time. After precipitating the protected peptide in H₂O and washing with diethyl ether, the solid was solved ACN—H₂O (1:1) and lyophilized to yield 1.266 g (82.4% recovery yield) Fmoc-¹³Gln(Trt)-Met-Glu(tBu)-Glu(tBu)-Glu(tBu)-¹⁵Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ as a white powder with a purity of 49.7% (evaluated after removal of side chain protecting group, treatment of peptide with TFA-TIS-H₂O (0.95:0.025:0.025) for 1 hour).

### Comparative Example 24d

### Solution Phase Synthesis of H-¹³Gln(Trt)-Met-Glu(tBu)-Glu(tBu)-Glu(tBu)-¹⁸Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 29)

The synthesis of H-¹³Gln(Trt)-Met-Glu(tBu)-Glu(tBu)-Glu(tBu)-¹⁸Ala-Val-Arg(Pbf)-Leu-Phe-Ile-Glu(tBu)-²⁵Trp(Boc)-Leu-Lys(Boc)-Asn(Trt)-Gly-Gly-³¹ Pro-Ser(tBu)-Ser(tBu)-Gly-³⁵Ala-Pro-Pro-Pro-³⁹Ser(tBu)-NH₂ was accomplished analogously to example 23d, the starting material peptide was prepared according to in example 24c (400.3 mg, 1.0 eq), affording 380 mg (quantitative yield) of H-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ as a white to beige powder with 37.19% purity (evaluated after removal of side chain protecting group, treatment of peptide with TFA-TIS-H₂O (0.95:0.025:0.025) for 1 hour).

### Comparative Example 24e

### Solution Phase Synthesis of Boc-¹His(Trt)-Gly-Glu(tBu)-⁴Gly-PsiThr-Phe-Thr(tBu)-⁸Ser(tBu)-Asp(tBu)-Leu-PsiSer-Lys(Boc)-¹³Gln(Trt)-Met-Glu(tBu)-Glu(tBu)-Glu(tBu)-¹⁸Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 1)

The peptide fragment Boc-¹His(Trt)-Gly-Glu(tBu)-⁴Gly-PsiThr-Phe-Thr(tBu)-⁸Ser(tBu)-Asp(tBu)-Leu-PsiSer-¹²Lys(Boc)-OH (SEQ ID NO 28) (167 mg, 1.0 eq; prepared according to example 24b) and HOBt (12.5 mg, 1.0 eq) were solved in DMF (1.2 ml). The PyBOP (55.5 mg, 1.3 eq) solved in DMF (0.2 ml) was added to the reaction mixture at low temperature and the pH was adjusted to 8-9 with DIPEA. After 5 minutes of acid activation, the DMF (2 ml) solved peptide fragment H-¹³Gln(Trt)-Met-Glu(tBu)-Glu(tBu)-Glu(tBu)-¹⁸Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂(SEQ ID NO 29) (350.0 mg, 1.0 eq; prepared according to example 24d) was added to the reaction mixture. The temperature of reaction was controlled to 0-5° C. for first 4 hours and then the reaction was allowed to go to completion at room temperature. The reaction was monitored by HPLC and after 22 hours the expected peptide was no detected and PyBOP (55.5 mg, 1.3 eq) was added to the reaction mixture and the pH was adjusted to 8-9 with DIPEA. The reaction was controlled after a total reaction time of 48 hours and 4 days.

After precipitating the protected peptide in H₂O and washing with diethyl ether the solid was solved ACN—H₂O (1:1) and lyophilized, HPLC did not show the formation of Exenatide sequence, just both starting material peptide sequences were detected (H-¹His-Gly-Glu-⁴Gly-Thr-Phe-Thr-⁸Ser-Asp-Leu-Ser-¹²Lys(Boc)-OH and H-¹³Gln-Met-Glu-Glu-Glu-¹⁸Ala-Val-Arg-Leu-Phe-Ile-Glu-²⁵Trp-Leu-Lys-Asn-Gly-Gly-³¹Pro-Ser-Ser-Gly-³⁵Ala-Pro-Pro-Pro-³⁹Ser-NH₂, evaluated after removal of side chain protecting group, treatment of peptide with TFA-TIS-H₂O (0.95:0.025:0.025) for 1 hour).

### Example 25a and Example 26a

### Solid Phase Synthesis of Fmoc-²⁷Lys(Boc)-Asn(Trt)-²⁹Gly-Gly-Pro-Ser(tBu)-Ser(tBu)-³⁴Gly-Ala-Pro-Pro-³⁸Pro-OH (SEQ ID NO 24)

The first steps of synthesis were carried out manually in a 60 ml solid phase reactor. Fmoc-Pro-OH (1.06 g, 0.5 eq) was loaded onto CTC resin (6 g, 1.55 mmol/g) in presence of DIPEA (5.101 ml, 10.0 eq relative to the amino acid) in DCM (minimum quantity to solve the amino acid, 8 ml). The reaction was finished after 60 minutes. After completion, the unreacted active positions on the resin were then capped by reaction with excess of methanol (4.8 ml) added directly to the reaction mixture. The bed was drained, and thoroughly washed with DCM (3 times 40 ml) and DMF (3 times 40 ml). Removal of the Fmoc group was accomplished at room temperature by using a solution of piperidine in DMF (20% piperidine; 1 time 1 min; 2 times 5 min; 40 ml each); four to five cycles were carried out. The bed was drained, and the H-Pro-CTC resin obtained was washed with DMF (3 times 40 ml) and DCM (3 times 40 ml) to remove residual piperidine. The dibenzofulvene formed as a product of Fmoc removal was quantified by UV and its value is directly related with the real loading of resin (0.50 mmol/g). The following steps to complete the peptide synthesis were performed in a mid-scale peptide synthesizer automated solid phase synthesis. The equipment runs using the same standard conditions to introduce every amino acid. A solution of Fmoc-Pro-OH (5.061 g, 5.0 eq) in DMF (20 ml) was added, and the coupling accomplished at room temperature in the presence of HBTU (5.689 g, 5.0 eq) and DIPEA (10.0 eq, pH=7-8). The coupling reaction time was fixed in 60 minutes at room temperature. After complete coupling, the obtained Fmoc-Pro-Pro-CTC resin was drained and thoroughly washed with DMF (3 times 40 ml) and DCM (3 times 40 ml).

The Fmoc group was removed as described after the introduction of the first amino acid on the resin.

The elongation cycle (Fmoc removal and amino acid coupling with coupling reagent HBTU (5.689 g, 5.0 eq) and DIPEA (10.0 eq)) was repeated for subsequent assembly of the peptide fragment using 5.0 eq each of Fmoc-Pro-OH, Fmoc-Ala-OH, Fmoc-Gly-OH, Fmoc-Ser(tBu)-OH, Fmoc-Ser(tBu)-OH, Fmoc-Pro-OH, Fmoc-Gly-OH, Fmoc-Gly-OH, Fmoc-Asn(Trt)-OH and Fmoc-Lys(Boc)-OH.

After the last elongation cycle, the protected peptide was washed with DCM (3 times 40 ml) and cleaved from the resin by adding 2% TFA in DCM (8 times 4 ml). After filtration, the solution of the peptide was evaporated partially under reduced pressure and the peptide was precipitated with diethyl ether. Centrifugation afforded the solid peptide which was successively washed with diethyl ether. After the diethyl ether washes the solid was dissolved in ACN—H₂O (1:1) and lyophilized affording 4.8 g of Fmoc-²⁷Lys(Boc)-Asn(Trt)-²⁹Gly-Gly-Pro-Ser(tBu)-Ser(tBu)-³⁴Gly-Ala-Pro-Pro-³⁸Pro-OH (SEQ ID NO 24) as a white to beige powder with 73.35% purity.

### Comparative Example 25b

### Solid Phase Synthesis of Fmoc-¹³Gln(Trt)-Met-Glu(tBu)-Glu(tBu)-Glu(tBu)-¹⁸Ala-Val-Arg(Pbf)-Leu-Phe-²³Ile-Glu(tBu)-Trp(Boc)-²⁶Leu-OH (SEQ ID NO 30)

The synthesis was carried out manually in a 60 ml solid phase reactor. Fmoc-Leu-OH (0.53 g, 0.5 eq) was loaded onto CTC resin (3 g, 1.55 mmol/g) in presence of DIPEA (2.55 ml, 10.0 eq relative to the amino acid) in DCM (minimum quantity to solve the amino acid, 4 ml). The reaction was finished after 60 minutes. After completion, the unreacted active positions on the resin were then capped by reaction with excess of methanol (2.4 ml) added directly to the reaction mixture. The bed was drained, and thoroughly washed with DCM (3 times 30 ml) and DMF (3 times 30 ml) DCM and DMF. Removal of the Fmoc group was accomplished at room temperature by using a solution of piperidine in DMF (20% piperidine; 1 time 1 min; 2 times 5 min; 30 ml each); four to five cycles were carried out. The bed was drained, and the H-Leu-CTC resin obtained was washed with DMF (3 times 30 ml) and DCM (3 times 30 ml) to remove residual piperidine. The dibenzofulvene formed as a product of Fmoc removal was quantified by UV and its value is directly related with the real loading of resin (0.50 mmol/g). The elongation cycle (Fmoc removal and amino acid coupling with coupling reagent HOBt (0.690 g, 3.0 eq) and DIPCDI (0.7 ml, 3.0 eq)) was repeated for subsequent assembly of the peptide fragment using 3.0 eq each of Fmoc-Trp(Boc)-OH, Fmoc-Glu(tBu)-OH, Fmoc-Ile-OH, Fmoc-Phe-OH, Fmoc-Leu-OH, Fmoc-Arg(Pbf)-OH, Fmoc-Val-OH, Fmoc-Ala-OH, Fmoc-Glu(tBu)-OH, Fmoc-Glu(tBu)-OH, Fmoc-Glu(tBu)-OH, Fmoc-Met-OH, Fmoc-Gln(Trt)-OH. The coupling reactions time was 60 minutes and its completeness were determined by the ninhydrin test. The amino acids of the fragment sequence [¹³Gln(Trt)-Met-Glu(tBu)-Glu(tBu)-¹⁷Glu(tBu)] present steric hindrance and its introduction needed a more effective coupling reagent COMU (1.79 g, 3.0 eq) and DIPEA (1.530 ml, 3.0 eq) and more than one coupling treatment.

After the last elongation cycle, the protected peptide was washed with DCM (3 times 30 ml) and cleaved from the resin by adding 2% TFA in DCM (13 times 4 ml). After filtration, the solution of the peptide was evaporated partially under reduced pressure and the peptide was precipitated with diethyl ether. Centrifugation afforded the solid peptide which was successively washed with diethyl ether. After the diethyl ether washes the solid was dissolved in ACN—H₂O (1:1) and lyophilized affording 3.02 g of Fmoc-13Gln(Trt)-Met-Glu(tBu)-Glu(tBu)-Glu(tBu)-18Ala-Val-Arg(Pbf)-Leu-Phe-23Ile-Glu(tBu)-Trp(Boc)-²⁶Leu-OH (SEQ ID NO 30) as a white to beige powder with 75.50% purity (evaluated after removal of side chain protecting group, treatment of peptide with TFA-TIS-H₂O (0.95:0.025:0.025) for 1 hour).

### Example 25c and Example 26c

### Solution Phase Synthesis of Fmoc-²⁷Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 16)

HBTU (24 mg, 1.1 eq) and DIPEA (23.7 microliter, 2.0 eq) were added to Fmoc-²⁷Lys(Boc)-Asn(Trt)-Gly-Gly-³¹Pro-Ser(tBu)-Ser(tBu)-³⁴Gly-Ala-Pro-Pro-³⁸Pro-OH (SEQ ID NO 24) (100 mg, 1.0 eq; prepared according to example 25a or 26a) in DCM (1 ml). After pre-activation, the H-³⁹Ser(tBu)-NH₂ (10.1 mg, 1.1 eq) was added at room temperature. The pH was adjusted to pH 8-9 with DIPEA. The completion of the reaction was monitored by HPLC and the reaction was finished after 48 hours.

The solution is extracted consecutively with three times with 1N HCl (3 times 10 ml), H₂O (3 times 10 ml), sodium bicarbonate (3 times 10 ml) and H₂O (3 times 10 ml). The organic phase is evaporated under reduced pressure, solved and lyophilized to yield 108.0 mg (85% recovery yield) of Fmoc-²⁷Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 16) as a white powder with a purity of 89.92%.

The same protocol followed on a 400 mg scale of Fmoc-²⁷Lys(Boc)-Asn(Trt)-Gly-Gly-³¹Pro-Ser(tBu)-Ser(tBu)-³⁴Gly-Ala-Pro-Pro-³⁸Pro-OH (SEQ ID NO 24) was followed and the same results were obtained. It is therefore possible to produce more quantity obtaining the same results.

### Example 25d and Example 26d

### Solution Phase Synthesis of H-²⁷Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 16)

The synthesis of H-²⁷Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ was accomplished analogously to example 23d, the starting material peptide was prepared according to example 25c or 26c (401 mg, 1.0 eq), affording 353.5 mg (quantitative yield) of H-²⁷Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ as a white to beige powder with a purity of 84.79%.

### Comparative Example 25e

### Solution Phase Synthesis of Fmoc-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 29)

The peptide fragment Fmoc-¹³Gln(Trt)-Met-Glu(tBu)-Glu(tBu)-Glu(tBu)-¹⁸Ala-Val-Arg(Pbf)-Leu-Phe-²³Ile-Glu(tBu)-Trp(Boc)-²⁶Leu-OH (SEQ ID NO 30) (200 mg, 1.0 eq; prepared according to example 25b) and HOBt (10.8 mg, 1.0 eq) were solved in DMF (2 ml). The PyBOP (47.7 mg, 1.3 eq) solved in DMF (0.2 ml) was added to the reaction mixture at low temperature and the pH was adjusted to 8-9 with DIPEA. After 5 minutes of acid activation, the DMF (4 ml) solved peptide fragment H-²⁷Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 16) (117.2 mg, 1.0 eq; prepared according to example 25d, 26d) was added to the reaction mixture. The temperature of reaction was controlled to 0-5° C. for first 4 hours and then the reaction was allowed to go to completion at room temperature. The reaction was monitored by HPLC and 22 hours was the total reaction time. After precipitating the protected peptide in H₂O and washing with diethyl ether the solid was solved ACN—H₂O (1:1) and lyophilized to yield 193.5 mg (64% recovery yield) Fmoc-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ as a white powder with a purity of 44.1% (evaluated after removal of side chain protecting group, treatment of peptide with TFA-TIS-H₂O (0.95:0.025:0.025) for 1 hour).

### Comparative Example 25f

### Solution Phase Synthesis of H-¹³Gln(Trt)-Met-Glu(tBu)-Glu(tBu)-Glu(tBu)-18Ala-Val-Arg(Pbf)-Leu-Phe-Ile-Glu(tBu)-Trp(Boc)-26Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 29)

The synthesis of H-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ was accomplished analogously to example 23d, the starting material peptide Fmoc-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ was prepared according to example 25e (232.3 mg, 1.0 eq), affording 218.5 mg (99% recovery yield) of H-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ as a white to beige powder with 46.5% purity (evaluated after removal of side chain protecting group, treatment of peptide with TFA-TIS-H₂O (0.95:0.025:0.025) for 1 hour).

### Example 25g

### Solution Phase Synthesis of Boc-¹His(Trt)-Gly-Glu(tBu)-⁴Gly-PsiThr-Phe-Thr(tBu)-⁸Ser(tBu)-Asp(tBu)-Leu-PsiSer-Lys(Boc)-¹³Gln(Trt)-Met-Glu(tBu)-Glu(tBu)-Glu(tBu)-¹⁸Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 1)

The peptide fragment Boc-¹His(Trt)-Gly-Glu(tBu)-⁴Gly-PsiThr-Phe-Thr(tBu)-⁸Ser(tBu)-Asp(tBu)-Leu-PsiSer-¹²Lys(Boc)-OH (SEQ ID NO 28) (69.4 mg, 1.0 eq; prepared according to example 24b) and HOBt (5.4 mg, 1.0 eq) were solved in DMF (0.9 ml). The PyBOP (23.3 mg, 1.3 eq) solved in DMF (0.1 ml) was added to the reaction mixture at low temperature and the pH was adjusted to 8-9 with DIPEA. After 5 minutes of acid activation, the DMF (2.5 ml) solved peptide fragment H-¹³Gln(Trt)-Met-Glu(tBu)-Glu(tBu)-Glu(tBu)-¹⁸Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-N H2 (SEQ ID NO 29) (145.7 mg, 1.0 eq; prepared according to example 25f) was added to the reaction mixture. The temperature of reaction was controlled to 0-5° C. for first 4 hours and then the reaction was allowed to go to completion at room temperature. The reaction was monitored by HPLC and after 22 hours the expected peptide was no detected and PyBOP (23.3 mg, 1.3 eq) was added to the reaction mixture and the pH was adjusted to 8-9 with DIPEA. The reaction was controlled after a total reaction time of 48 hours.

After precipitating the protected peptide in H₂O and washing with diethyl ether the solid was solved ACN—H₂O (1:1) and lyophilized, HPLC did not show the formation of Exenatide sequence, just both starting material peptide sequences were detected (H-¹His-Gly-Glu-⁴Gly-Thr-Phe-Thr-⁸Ser-Asp-Leu-Ser-¹²Lys(Boc)-OH and H-¹³Gln-Met-Glu-Glu-Glu-¹⁸Ala-Val-Arg-Leu-Phe-Ile-Glu-²⁵Trp-Leu-Lys-Asn-Gly-Gly-³¹Pro-Ser-Ser-Gly-³⁵Ala-Pro-Pro-Pro-³⁹Ser-NH₂, evaluated after removal of side chain protecting group, treatment of peptide with TFA-TIS-H₂O (0.95:0.025:0.025) for 1 hour).

### Example 26b

### Solid Phase Synthesis of Fmoc-¹¹Ser(tBu)-Lys(Boc)-Gln(Trt)-Met-Glu(tBu)-¹⁶Glu(tBu)-Glu(tBu)-Ala-Val-Arg(Pbf)-²²Leu-Phe-Ile-Glu(tBu)-Trp(Boc)-²⁶Leu-OH (SEQ ID NO 13)

The synthesis of that peptide fragment was accomplished analogously to example 25b affording 2.37 g of Fmoc-¹¹Ser(tBu)-Lys(Boc)-Gln(Trt)-Met-Glu(tBu)-¹⁶Glu(tBu)-Glu(tBu)-Ala-Val-Arg(Pbf)-²²Leu-Phe-Ile-Glu(tBu)-Trp(Boc)-²⁶Leu-OH as a white to beige powder with 70.60% purity (evaluated after removal of side chain protecting group, treatment of peptide with TFA-TIS-H₂O (0.95:0.025:0.025) for 1 hour).

### Example 26e

### Solution Phase Synthesis of Fmoc-¹¹Ser(tBu)-Lys(Boc)-Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 9)

The peptide fragment Fmoc-¹¹Ser(tBu)-Lys(Boc)-Gln(Trt)-Met-Glu(tBu)-Glu(tBu)-Glu(tBu)-¹⁸Ala-Val-Arg(Pbf)-Leu-Phe-²³Ile-Glu(tBu)-Trp(Boc)-²⁶Leu-OH (SEQ ID NO 13) (200 mg, 1.0 eq; prepared according to example 26b) and HOBt (9.5 mg, 1.0 eq) were solved in DMF (4 ml). The PyBOP (42.0 mg, 1.3 eq) solved in DMF (0.2 ml) was added to the reaction mixture at low temperature and the pH was adjusted to 8-9 with DIPEA. After 5 minutes of acid activation, the DMF (4 ml) solved peptide fragment H-²⁷Lys(Boc)-Asn(Trt)-Gly-Gly-³¹Pro-Ser(tBu)-Ser(tBu)-Gly-³⁶Ala-Pro-Pro-Pro-³⁹Ser(tBu)-N H2 (SEQ ID NO 16) (117.2 mg, 1.0 eq; prepared according to example 25d or 26d) was added to the reaction mixture. The temperature of reaction was controlled to 0-5° C. for first 4 hours and then the reaction was allowed to go to completion at room temperature. The reaction was monitored by HPLC and 22 hours was the total reaction time. After precipitating the protected peptide in H₂O and washing with diethyl ether the solid was solved ACN—H₂O (1:1) and lyophilized to yield 188.9 mg (62.5% recovery yield) Fmoc-¹¹Ser(tBu)-Lys(Boc)-Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁶Ala-Pro-Pro-Pro-³⁹Ser(tBu)-NH₂ as a white powder with a purity of 35.86%.

### Example 27a and Example 28a

### Solid Phase Synthesis of Fmoc-²⁶Leu-Lys(Boc)-Asn(Trt)-²⁹Gly-Gly-Pro-Ser(tBu)-Ser(tBu)-³⁴Gly-Ala-Pro-Pro-³⁸Pro-OH (SEQ ID NO 23)

The first steps of synthesis were carried out manually in a 60 ml solid phase reactor. Fmoc-Pro-OH (0.744 g, 0.5 eq) was loaded onto CTC resin (4.1 g, 1.55 mmol/g) in presence of DIPEA (3.562 ml, 10.0 eq relative to the amino acid) in DCM (minimum quantity to solve the amino acid, 4 ml). The reaction was finished after 60 minutes. After completion, the unreacted active positions on the resin were then capped by reaction with excess of methanol (3.35 ml) added directly to the reaction mixture. The bed was drained, and thoroughly washed with DCM (3 times 30 ml) and DMF (3 times 30 ml). Removal of the Fmoc group was accomplished at room temperature by using a solution of piperidine in DMF (20% piperidine; 1 time 1 min; 2 times 5 min; 30 ml each); four to five cycles were carried out. The bed was drained, and the H-Pro-CTC resin obtained was washed with DMF (3 times 30 ml) and DCM (3 times 30 ml) to remove residual piperidine. The dibenzofulvene formed as a product of Fmoc removal was quantified by UV and its value is directly related with the real loading of resin (0.59 mmol/g). The two following Pro were coupled manually, to avoid Pro deletion detected in first synthesis approach. Both amino acids were coupled as a solution of Fmoc-Pro-OH (2.23 g, 3.0 eq) in DMF (10 ml), and the coupling accomplished at room temperature in the presence of oxyma (0.89 g, 3.0 eq) and DIPCDI (0.973 ml, 3.0 eq). The coupling reaction time was 60 minutes and its completeness was determined by the ninhydrin test. After coupling two consecutive coupling of Pro, the obtained Fmoc-Pro-Pro-Pro-CTC resin was drained and thoroughly washed with DMF (3 times 30 ml) and DCM (3 times 30 ml).

The following steps to complete the peptide synthesis were performed in a mid-scale peptide synthesizer automated solid phase synthesis. The equipment runs using the same standard conditions to introduce every amino acid. A solution of Fmoc-Ala-OH (4.670 g, 5.0 eq) in DMF (20 ml) was added, and the coupling accomplished at room temperature in the presence of HBTU (3.793 g, 5.0 eq) and DIPEA (10.0 eq, pH=7-8). The coupling reaction time was fixed in 60 min at room temperature. After complete coupling, the obtained Fmoc-Ala-Pro-Pro-Pro-CTC resin was drained and thoroughly washed with DMF (3 times 30 ml) and DCM (3 times 30 ml).

The Fmoc group was removed as described after the introduction of the first amino acid on the resin.

The elongation cycle (Fmoc removal and amino acid coupling with coupling reagent HBTU (3.793 g, 5.0 eq) and DIPEA (10.0 eq)) was repeated for subsequent assembly of the peptide fragment using 5.0 eq each of Fmoc-Gly-OH, Fmoc-Ser(tBu)-OH, Fmoc-Ser(tBu)-OH, Fmoc-Pro-OH, Fmoc-Gly-OH, Fmoc-Gly-OH, Fmoc-Asn(Trt)-OH, Fmoc-Lys(Boc)-OH and Fmoc-Leu-OH.

After the last elongation cycle, the protected peptide was washed with DCM (3 times 30 ml) and cleaved from the resin by adding 2% TFA in DCM (8 times 4 ml). After filtration, the solution of the peptide was evaporated partially under reduced pressure and the peptide was precipitated with diethyl ether. Centrifugation afforded the solid peptide which was successively washed with diethyl ether. After the diethyl ether washes the solid was dissolved in ACN—H₂O (1:1) and lyophilized affording 3.28 g of Fmoc-²⁶Leu-Lys(Boc)-Asn(Trt)-²⁹Gly-Gly-Pro-Ser(tBu)-Ser(tBu)-³⁴Gly-Ala-Pro-Pro-³⁸Pro-OH (SEQ ID NO 23) as a white to beige powder with 93.4% purity.

### Example 27b

### Solid Phase Synthesis of Fmoc-¹¹Ser(tBu)-Lys(Boc)-Gln(Trt)-Met-Glu(tBu)-¹⁶Glu(tBu)-Glu(tBu)-Ala-Val-²⁰Arg(Pbf)-Leu-Phe-Ile-Glu(tBu)-²⁶Trp(Boc)-OH (SEQ ID NO 14)

The synthesis was carried out in a 60 ml solid phase reactor. The portion of Fmoc-[13-25]-CTC synthesized in example 28b was elongated by cycle (Fmoc removal and amino acid coupling with coupling reagent COMU (1.790 g, 3.0 eq) and DIPEA (6.0 eq, pH=7-8) was repeated for subsequent assembly of the peptide fragment using 3.0 eq each of Fmoc-Lys(Boc)-OH and Fmoc-Ser(tBu)-OH. The coupling reaction time was 60 minutes and its completeness was determined by the ninhydrin test. The bed was drained, and thoroughly washed with DCM (3 times 30 ml) and DMF (3 times 30 ml).

After the last elongation cycle, the protected peptide was washed with DCM (3 times 30 ml) and cleaved from the resin by adding 2% TFA in DCM (13 times 4 ml). After filtration, the solution of peptide was evaporated partially under reduced pressure and the peptide was precipitated with diethyl ether. Centrifugation afforded the solid peptide which was successively washed with diethyl ether. After the diethyl ether washes the solid was dissolved in ACN—H₂O (1:1) and lyophilized affording 1.87 g of Fmoc-¹¹Ser(tBu)-Lys(Boc)-Gln(Trt)-Met-Glu(tBu)-¹⁶Glu(tBu)-Glu(tBu)-Ala-Val-²⁰Arg(Pbf)-Leu-Phe-Ile-Glu(tBu)-²⁶Trp(Boc)-OH (SEQ ID NO 14) as a white to beige powder with 69.60% purity (evaluated after removal of side chain protecting group, treatment of peptide with TFA-TIS-H₂O (0.95:0.025:0.025) for 1 hour).

### Example 27c and Example 28c

### Solution Phase Synthesis of Fmoc-²⁶Leu-Lys(Boc)-Asn(Trt)-Gly-Gly-³¹Pro-Ser(tBu)-Ser(tBu)-Gly-³⁶Ala-Pro-Pro-Pro-³⁹Ser(tBu)-NH₂ (SEQ ID NO 15)

HBTU (22.4 mg, 1.1 eq) and DIPEA (23.7 microliter, 2.5 eq) were added to Fmoc-²⁶Leu-Lys(Boc)-Asn(Trt)-Gly-Gly-³¹Pro-Ser(tBu)-Ser(tBu)-Gly-³⁵Ala-Pro-Pro-³⁸Pro-OH (SEQ ID NO 23) (100 mg, 1.0 eq; prepared according to example 27a or 28a) in DCM (1 ml). After pre-activation, the H-³⁹Ser(tBu)-NH₂ (9.5 mg, 1.1 eq) was added at room temperature. The pH was adjusted to pH 8-9 with DIPEA. The completion of the reaction was monitored by HPLC and the reaction was finished after 19 hours.

The solution is extracted consecutively with three times with 1N HCl (3 times 10 ml), H₂O (3 times 10 ml), sodium bicarbonate (3 times 10 ml) and H₂O (3 times 10 ml). The organic phase is evaporated under reduced pressure, solved and lyophilized to yield 83.6 mg (78% recovery yield quantitative yield) of Fmoc-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 15) as a white powder with a purity of 84.38% (evaluated after removal of side chain protecting group, treatment of peptide with TFA-TIS-H₂O (0.95:0.025:0.025) for 1 hour).

The same protocol followed on a 400 mg scale of Fmoc-²⁶Leu-Lys(Boc)-Asn(Trt)-Gly-Gly-³¹Pro-Ser(tBu)-Ser(tBu)-Gly-³⁵Ala-Pro-Pro-³⁸Pro-OH (SEQ ID NO 23) was followed and the same results were obtained. It is therefore possible to produce more quantity obtaining the same results.

### Example 27d and Example 28d

### Solution Phase Synthesis of H-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 15)

The synthesis of H-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ was accomplished analogously to example 23d, the starting material peptide was prepared according to example 27c or 28c (400 mg, 1.0 eq), affording 355.5 mg (99% recovery yield) of H-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ as a white to beige powder with 91.30% purity (evaluated after removal of side chain protecting group, treatment of peptide with TFA-TIS-H₂O (0.95:0.025:0.025) for 1 hour).

### Example 27e

### Solution Phase Synthesis of Fmoc-¹¹Ser(tBu)-Lys(Boc)-Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁸Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 9)

The peptide fragment Fmoc-¹¹ Ser(tBu)-Lys(Boc)-Gln(Trt)-Met-Glu(tBu)-Glu(tBu)-Glu(tBu)-¹⁸Ala-Val-Arg(Pbf)-Leu-Phe-²³Ile-Glu(tBu)-²⁵Trp(Boc)-OH (SEQ ID NO 14) (200 mg, 1.0 eq; prepared according to example 27b) and HOBt (9.8 mg, 1.0 eq) were solved in DMF (3 ml). The PyBOP (44.0 mg, 1.3 eq) solved in DMF (0.2 ml) was added to the reaction mixture at low temperature and the pH was adjusted to 8-9 with DIPEA. After 5 minutes of acid activation, the DMF (4 ml) solved peptide fragment H-²⁸Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-N H2 (SEQ ID NO 15) (103.2 mg, 1.0 eq; prepared according to example 27d or 28d) was added to the reaction mixture. The temperature of reaction was controlled to 0-5° C. for first 4 hours and then the reaction was allowed to go to completion at room temperature. The reaction was monitored by HPLC and 22 hours was the total reaction time. After precipitating the protected peptide in H₂O and washing with diethyl ether the solid was solved ACN—H₂O (1:1) and lyophilized to yield 263 mg (84% recovery yield) Fmoc-¹¹Ser(tBu)-Lys(Boc)-Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁸Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ as a white powder with a purity of 37.32%.

### Comparative Example 28b

### Solid Phase Synthesis of Fmoc-¹³Gln(Trt)-Met-Glu(tBu)-Glu(tBu)-¹⁷Glu(tBu)-Ala-Val-Arg(Pbf)-²¹Leu-Phe-Ile-Glu(tBu)-²⁵Trp(Boc)-OH (SEQ ID NO 31)

The synthesis of that peptide fragment was accomplished analogously to example 25b onto a CTC resin (6 g, 1.55 mmol/g) with the same problems as encoutered in example 25b. The resin was divided in two equal portions and one of them was used in example 27b and the second one was cleaved from the resin affording 2.44 g of Fmoc-13Gln(Trt)-Met-Glu(tBu)-Glu(tBu)-¹⁷Glu(tBu)-Ala-Val-Arg(Pbf)-²¹Leu-Phe-Ile-Glu(tBu)-²⁵Trp(Boc)-OH as a white to beige powder with 50.69% purity (evaluated after removal of side chain protecting group, treatment of peptide with TFA-TIS-H₂O (0.95:0.025:0.025) for 1 hour).

### Comparative Example 28e

### Solution Phase Synthesis of Fmoc-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-N H₂ (SEQ ID NO 29)

The peptide fragment Fmoc-¹³Gln(Trt)-Met-Glu(tBu)-Glu(tBu)-Glu(tBu)-¹⁸Ala-Val-Arg(Pbf)-Leu-Phe-²³Ile-Glu(tBu)-²⁵Trp(Boc)-OH (SEQ ID NO 31) (182.4 mg, 1.0 eq; prepared according to example 28b) and HOBt (10.3 mg, 1.0 eq) were solved in DMF (4 ml). The PyBOP (49.8 mg, 1.3 eq) solved in DMF (0.2 ml) was added to the reaction mixture at low temperature and the pH was adjusted to 8-9 with DIPEA. After 5 minutes of acid activation, the DMF (4 ml) solved peptide fragment H-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 15) (103.2 mg, 1.0 eq; prepared according to example 27d or 28d) was added to the reaction mixture. The temperature of reaction was controlled to 0-5° C. for first 4 hours and then the reaction was allowed to go to completion at room temperature. The reaction was monitored by HPLC and 22 hours was the total reaction time. After precipitating the protected peptide in H₂O and washing with diethyl ether the solid was solved ACN—H₂O (1:1) and lyophilized to yield 140 mg (60.76% recovery yield) Fmoc-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ as a white powder with a purity of 33.2%.

### Comparative Example 28f

### Solution Phase Synthesis of H-¹³Gln(Trt)-Met-Glu(tBu)-Glu(tBu)-Glu(tBu)-18Ala-Val-Arg(Pbf)-Leu-Phe-Ile-Glu(tBu)-Trp(Boc)-26Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 29)

The synthesis of H-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ was accomplished analogously to example 23d, the starting material peptide was prepared according to example 28e (74 mg, 1.0 eq), affording 50 mg (71% recovery yield) of H-¹³Gln(Trt)-Met-Glu(tBu)-Glu(tBu)-Glu(tBu)-¹⁸Ala-Val-Arg(Pbf)-Leu-Phe-Ile-Glu(tBu)-Trp(Boc)-²⁶Leu-Lys(Boc)-Asn(Trt)-Gly-Gly-³¹ Pro-Ser(tBu)-Ser(tBu)-Gly-³⁵Ala-Pro-Pro-Pro-³⁹Ser(tBu)-NH₂ as a white to beige powder with 38.7% purity (evaluated after removal of side chain protecting group, treatment of peptide with TFA-TIS-H₂O (0.95:0.025:0.025) for 1 hour).

### Example 28g

### Solution Phase Synthesis of Boc-¹His(Trt)-Gly-Glu(tBu)-⁴Gly-PsiThr-Phe-Thr(tBu)-⁸Ser(tBu)-Asp(tBu)-Leu-PsiSer-Lys(Boc)-¹³Gln(Trt)-Met-Glu(tBu)-Glu(tBu)-Glu(tBu)-¹⁸Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂ (SEQ ID NO 1)

The peptide fragment Boc-¹His(Trt)-Gly-Glu(tBu)-⁴Gly-PsiThr-Phe-Thr(tBu)-⁸Ser(tBu)-Asp(tBu)-Leu-PsiSer-¹²Lys(Boc)-OH (SEQ ID NO 28) (66.6 mg, 1.0 eq; prepared according to example 24b) and HOBt (5.0 mg, 1.0 eq) were solved in DMF (0.9 ml). The PyBOP (22.3 mg, 1.3 eq) solved in DMF (0.1 ml) was added to the reaction mixture at low temperature and the pH was adjusted to 8-9 with DIPEA. After 5 minutes of acid activation, the DMF (2 ml) solved peptide fragment H-¹³Gln(Trt)-Met-Glu(tBu)-Glu(tBu)-Glu(tBu)-¹⁸Ala-Val-Arg(Pbf)-Leu-Phe-Ile-Glu(tBu)-²⁵Trp(Boc)-Leu-Lys(Boc)-Asn(Trt)-Gly-Gly-³¹Pro-Ser(tBu)-Ser(tBu)-Gly-³⁵Ala-Pro-Pro-Pro-³⁹Ser(tBu)-NH₂(SEQ ID NO 29) (140 mg, 1.0 eq; prepared according to example 28f) was added to the reaction mixture. The temperature of reaction was controlled to 0-5° C. for first 4 hours and then the reaction was allowed to go to completion at room temperature. The reaction was monitored by HPLC and after 22 hours the expected peptide was no detected and PyBOP (22.3 mg, 1.3 eq) was added to the reaction mixture and the pH was adjusted to 8-9 with DIPEA. The reaction was controlled after a total reaction time of 48 hours. After precipitating the protected peptide in H₂O and washing with diethyl ether the solid was solved ACN—H₂O (1:1) and lyophilized, HPLC did not show the formation of Exenatide sequence, just both starting material peptide sequences were detected (H-¹His-Gly-Glu-⁴Gly-Thr-Phe-Thr-⁸Ser-Asp-Leu-Ser-¹²Lys(Boc)-OH and H-¹³Gln-Met-Glu-Glu-Glu-¹⁸Ala-Val-Arg-Leu-Phe-Ile-Glu-²⁵Trp-Leu-Lys-Asn-Gly-Gly-³¹Pro-Ser-Ser-Gly-³⁵Ala-Pro-Pro-Pro-³⁹Ser-NH₂, evaluated after removal of side chain protecting group, treatment of peptide with TFA-TIS-H₂O (0.95:0.025:0.025) for 1 hour).

1. A method for the preparation of a peptide (1),
the peptide (1) being selected from the group consisting of peptide (2) and peptide (3),
the peptide (2) having the formula (la);
(Ia)
(SEQ ID NO 1)
H-¹His-Gly-Glu-Gly-⁵Thr-Phe-Thr-Ser-Asp-¹⁰Leu-
Ser-Lys-Gln-Met-¹⁵Glu-Glu-Glu--Ala-Val-²⁰Arg-
Leu-Phe-Ile-Glu-²⁵Trp-Leu-Lys-Asn-Gly-³⁰Gly-Pro-
Ser-Ser-Gly-³⁵Ala-Pro-Pro-Pro-Ser-NH₂
the peptide (3) having the formula (Ib);
(Ib)
(SEQ ID NO 2)
H-¹His-Gly-Glu-Gly-⁵Thr-Phe-Thr-Ser-Asp-¹⁰Leu-
Ser-Lys-Gln-Met-¹⁵Glu-Glu-Glu--Ala-Val-²⁰Arg-
Leu-Phe-Ile-Glu-²⁵Trp-Leu-Lys-Asn-Gly-³⁰Gly-Pro-
Ser-Ser-Gly-³⁵Ala-Pro-Pro-Ser-Lys-⁴⁰Lys-Lys-Lys-
Lys-Lys-NH₂
characterized by preparing the peptide (1) with a three-fragment-strategy from peptide fragments (A), (B) and (C) by solution phase synthesis,
the peptide fragment (B) being derived from peptide (1),
the peptide fragment (B) having as N-terminal amino acid the amino acid of position 11 of peptide (1); and
the peptide fragment (B) having as C-terminal amino acid the amino acid of position XB of peptide (1), with XB being 20, 21, 22, 23, 24, 25 or 26;
the peptide fragment (B) thereby having the sequence ^{XB}Ser to ^{XB}Xaa of peptide (1);
the peptide fragment (B) bearing a N-terminal protecting group PGB of the carbamate-type;
the peptide fragment (B) being side-chain protected,
with the proviso, that peptide fragment (B) has no pseudoproline;
the peptide fragment (A) having the formula (VII);
(VII),
(SEQ ID NO 11)
P3-¹His-Gly-Glu-Gly-⁵Thr-Phe-Thr-Ser-Asp-¹⁰Leu-OH
wherein P3 is a carbamate-type protecting group,
the peptide fragment (C) being selected from the group consisting of peptide fragments (CX1-Y1),
the peptide fragments (CX1-Y1) being derived from peptide (1),
X1 is XB+1, and X1 designating the N-terminal amino acid of peptide fragment (C), which is the amino acid of position X1 of peptide (1), and
Y1 is 39 or 44 and designates the C-terminal amino acid of peptide fragment (C), which is the amino acid 39 of peptide (2) or the amino acid 44 of peptide (3) respectively;
the peptide fragment (C) thereby having the sequence ^{X1}Xaa to ^{Y1}Xaa of peptide (1);
the peptide fragment (C) bearing no N-terminal protecting group;
the peptide fragment (C) being side-chain protected;
further characterized that
in a first step (c-ex) the peptide fragment (B) is coupled with the peptide fragment (C),
resulting in a peptide fragment (D) bearing an N-terminal protecting group PGB;
the peptide fragment (D) being a peptide fragment (D2) or a peptide fragment (D3),
the peptide fragment (D2) having the amino acid sequence (SEQ ID NO 9) and the formula (VIaa),
(VIaa),
(SEQ ID NO 9)
PGB-Ser-Lys-Gln-Met-¹⁵Glu-Glu-Glu-Ala-Val-²⁰Arg-
Leu-Phe-IIe-Glu-²⁵Trp-Leu--Lys-Asn-Gly-³⁰Gly-Pro-
Ser-Ser-Gly-³⁵Ala-Pro-Pro-Pro-Ser-NH₂
the peptide fragment (D3) having the amino acid sequence (SEQ ID NO 10) and having the formula (VIba),
(VIba),
(SEQ ID NO 10)
PGB-Ser-Lys-Gln-Met-¹⁵Glu-Glu-Glu-Ala-Val-²⁰Arg-
Leu-Phe-Ile-Glu-²⁵Trp-Leu--Lys-Asn-Gly-³⁰Gly-Pro-
Ser-Ser-Gly-³⁵Ala-Pro-Pro-Ser-Lys-⁴⁰Lys-Lys-Lys-
Lys--Lys-NH₂
and then
in a second step (d-ex), the N-terminal protecting group PGB of the peptide fragment (D) is removed;
and then
in a third step (e-ex), the peptide fragment (D) is coupled with the peptide fragment (A) resulting in peptide (1) bearing a protecting group P3,
and then
in a fourth step (f-ex), the N-terminal protecting group P3 is removed from peptide (1),
and in this step (f-ex) or afterwards,the side chain protecting groups are removed from peptide (1).

2. A method for the preparation of a peptide (1) according to claim 1, wherein the peptide fragment (B) or the peptide fragment (A) are prepared by solid phase peptide synthesis.

3. A method for the preparation of a peptide (1) according to claim 1, wherein the peptide fragment (C) is prepared from a N-terminally protected peptide fragment (C),
which is N-terminally protected by a N-terminal protecting group PC,
and which is prepared by solution phase synthesis, by solid phase synthesis, or by a combination of solution phase synthesis and solid phase synthesis,
with PC being a carbamate type protecting group,
and subsequent removal of the N-terminal protecting group PC.

4. A method for the preparation of a peptide (1) according to claim 3, wherein the peptide fragment (C) is prepared by a step (a-ex), the step (a-ex) being a solution phase synthesis coupling of a peptide fragment (CL) with a peptide fragment (CR);
wherein
the peptide fragment (CL) being selected from the group consisting of peptide fragments (CLX1-Y2), which are derived from peptide (1), X1 being XB+1, and X1 designating the N-terminal amino acid of peptide fragment (C), which is the amino acid of position X1 of peptide (1);
Y2 is 29, 30 or 31 and designates the C-terminal amino acid of peptide fragment (CL), which is the amino acid Y2 of peptide (1);
the peptide fragment (CL) thereby having the sequence ^{X1}Xaa to ^{Y2}Xaa of peptide (1);
the peptide fragment (CL) bearing PC, with PC being a carbamate type protecting group, and subsequent removal of the N-terminal protecting group PC;
the peptide fragment (CL) being side-chain protected; and
wherein
the peptide fragment (CR) being selected from the group consisting of peptide fragments (CRX2-Y1), which are derived from peptide (1),
wherein
X2 is Y2+1 and designates the N-terminal amino acid of peptide fragment (CR), which is the amino acid of position X2 of peptide (1), and
Y1 being Y1 is 39 or 44 and designates the C-terminal amino acid of peptide fragment (C), which is the amino acid 39 of peptide (2) or the amino acid 44 of peptide (3) respectively;
the peptide fragment (CR) thereby having the sequence ^{X2}Xaa to ^{Y1}Xaa of peptide (1);
the peptide fragment (CR) bearing no N-terminal protecting group;
the peptide fragment (CR) being side-chain protected;
and subsequent removal of the N-terminal protecting group PC.

5. A method for the preparation of a peptide (1) according to claim 4, wherein the peptide fragment (CL) is prepared by solid phase peptide synthesis.

6. A method for the preparation of a peptide (1) according to claim 4, wherein the peptide fragment (CR) is prepared from a N-terminally protected peptide fragment (CR),
which is N-terminally protected by a N-terminal protecting group PC, with PC being a carbamate type protecting group,
and subsequent removal of the N-terminal protecting group PC,
and which is prepared by SPS, by SPPS, or by a combination of SPPS and SPS,
and subsequent removal of the N-terminal protecting group PC.

7. A method for the preparation of a peptide (1) according to claim 4, wherein X1 is 22.

8. A method for the preparation of a peptide (1) according to claim 7, wherein Y2 is 29.

9. A method for the preparation of a peptide (1) according to claim 6, wherein the peptide fragments (CRX2-Y1), which are derived from peptide (1), with Y1 being 39, are prepared by solution phase coupling of a peptide fragment (CRX2-(Y1-1)) with H-Ser-NH_{2;}
and
subsequent removal of PC.

10. A peptide fragment selected from the group consisting of peptide fragment (A), (B), (C), (D), (CL) and (CR), the peptide fragments (A), (B), (C) and (D) being as defined in claim 1 and the peptide fragment (CL) selected from the group consisting of peptide fragments (CLX1-Y2), which are derived from peptide (1),
X1 being XB+1, and X1 designating the N-terminal amino acid of peptide fragment (C), which is the amino acid of position X1 of peptide (1);
Y2 is 29, 30 or 31 and designates the C-terminal amino acid of peptide fragment (CL),
which is the amino acid Y2 of peptide (1);
the peptide fragment (CL) thereby having the sequence ^{X1}Xaa to ^{Y2}Xaa of peptide (1);
the peptide fragment (CL) bearing PC, with PC being a carbamate type protecting group,
and subsequent removal of the N-terminal protecting group PC;
the peptide fragment (CL) being side-chain protected; and
peptide fragment (CR) selected from the group consisting of peptide fragments (CRX2-Y1), which are derived from peptide (1),
wherein
X2 is Y2+1 and designates the N-terminal amino acid of peptide fragment (CR), which is the amino acid of position X2 of peptide (1), and
Y1 being Y1 is 39 or 44 and designates the C-terminal amino acid of peptide fragment (C), which is the amino acid 39 of peptide (2) or the amino acid 44 of peptide (3) respectively;
the peptide fragment (CR) thereby having the sequence ^{X2}Xaa to ^{Y1}Xaa of peptide (1);
the peptide fragment (CR) bearing no N-terminal protecting group;
the peptide fragment (CR) being side-chain protected;
and subsequent removal of the N-terminal protecting group PC.

11. A peptide fragment (B) according to claim 10, wherein XB is 21, 25 or 26.

12. A peptide fragment selected from the group consisting of peptide fragments (CLX1-Y2) and peptide fragments (CRX2-Y1), the peptide fragments (CLX1-Y2) and the peptide fragments (CRX2-Y1) being as defined in claim 4, with X1 being 22.

13. A peptide fragment according to claim 12, wherein Y2 is 29.

14. A peptide fragment (CR) selected from the group consisting of peptide fragments (CRX2-(Y1-1)), the peptide fragments (CRX2-(Y1-1)) begin as defined in claim 9.

15. A method for the preparation of a peptide (1) according to claim 2, wherein the peptide fragment (C) is prepared from a N-terminally protected peptide fragment (C),
which is N-terminally protected by a N-terminal protecting group PC,
and which is prepared by solution phase synthesis, by solid phase synthesis, or by a combination of solution phase synthesis and solid phase synthesis,
with PC being a carbamate type protecting group,
and subsequent removal of the N-terminal protecting group PC.


SEQUENCE LISTING (31 sequences)

SEQ ID NO: 1 - protein, 39 aa, Artificial sequence
  Synthetic construct
HGEGTFTSDLSKQMEEEAVRLFIEWLKNGGPSSGAPPPS

SEQ ID NO: 2 - protein, 44 aa, Artificial sequence
  Synthetic construct
HGEGTFTSDLSKQMEEEAVRLFIEWLKNGGPSSGAPPSKKKKKK

SEQ ID NO: 3 - protein, 8 aa, Artificial sequence
  Synthetic construct
FIEWLKNG

SEQ ID NO: 4 - protein, 10 aa, Artificial sequence
  Synthetic construct
GPSSGAPPPS

SEQ ID NO: 5 - protein, 15 aa, Artificial sequence
  Synthetic construct
GPSSGAPPSKKKKKK

SEQ ID NO: 6 - protein, 18 aa, Artificial sequence
  Synthetic construct
FIEWLKNGGPSSGAPPPS

SEQ ID NO: 7 - protein, 23 aa, Artificial sequence
  Synthetic construct
FIEWLKNGGPSSGAPPSKKKKKK

SEQ ID NO: 8 - protein, 11 aa, Artificial sequence
  Synthetic construct
SKQMEEEAVRL

SEQ ID NO: 9 - protein, 29 aa, Artificial sequence
  Synthetic construct
SKQMEEEAVRLFIEWLKNGGPSSGAPPPS

SEQ ID NO: 10 - protein, 34 aa, Artificial sequence
  Synthetic construct
SKQMEEEAVRLFIEWLKNGGPSSGAPPSKKKKKK

SEQ ID NO: 11 - protein, 10 aa, Artificial sequence
  Synthetic construct
HGEGTFTSDL

SEQ ID NO: 12 - protein, 9 aa, Artificial sequence
  Synthetic construct
GPSSGAPPP

SEQ ID NO: 13 - protein, 16 aa, Artificial sequence
  Synthetic construct
SKQMEEEAVRLFIEWL

SEQ ID NO: 14 - protein, 15 aa, Artificial sequence
  Synthetic construct
SKQMEEEAVRLFIEW

SEQ ID NO: 15 - protein, 14 aa, Artificial sequence
  Synthetic construct
LKNGGPSSGAPPPS

SEQ ID NO: 16 - protein, 13 aa, Artificial sequence
  Synthetic construct
KNGGPSSGAPPPS

SEQ ID NO: 17 - protein, 9 aa, Artificial sequence
  Synthetic construct
FIEWLKNGG

SEQ ID NO: 18 - protein, 9 aa, Artificial sequence
  Synthetic construct
PSSGAPPPS

SEQ ID NO: 19 - protein, 10 aa, Artificial sequence
  Synthetic construct
FIEWLKNGGP

SEQ ID NO: 20 - protein, 8 aa, Artificial sequence
  Synthetic construct
SSGAPPPS

SEQ ID NO: 21 - protein, 14 aa, Artificial sequence
  Synthetic construct
PSSGAPPSKKKKKK

SEQ ID NO: 22 - protein, 13 aa, Artificial sequence
  Synthetic construct
SSGAPPSKKKKKK

SEQ ID NO: 23 - protein, 13 aa, Artificial sequence
  Synthetic construct
LKNGGPSSGAPPP

SEQ ID NO: 24 - protein, 12 aa, Artificial sequence
  Synthetic construct
KNGGPSSGAPPP

SEQ ID NO: 25 - protein, 8 aa, Artificial sequence
  Synthetic construct
PSSGAPPP

SEQ ID NO: 26 - protein, 7 aa, Artificial sequence
  Synthetic construct
SSGAPPP

SEQ ID NO: 27 - protein, 17 aa, Artificial sequence
  Synthetic construct
QMEEEAVRLFIEWLKNG

SEQ ID NO: 28 - protein, 12 aa, Artificial sequence
  Synthetic construct
HGEGTFTSDLSK

SEQ ID NO: 29 - protein, 27 aa, Artificial sequence
  Synthetic construct
QMEEEAVRLFIEWLKNGGPSSGAPPPS

SEQ ID NO: 30 - protein, 14 aa, Artificial sequence
  Synthetic construct
QMEEEAVRLFIEWL

SEQ ID NO: 31 - protein, 13 aa, Artificial sequence
  Synthetic construct
QMEEEAVRLFIEW